(19) United States
(12) Patent Application Publication
(10) Pub. No.: US 2026/0217679 A1
(43) Pub. Date: Jul. 30, 2026

(54) CANCER TREATING COMPOUNDS

(71) Applicant: UNIVERSITY OF BRADFORD, Bradford (GB)

(72) Inventors: Robert FALCONER, Bradford (GB); David PAJTAS, Bradford (GB); Klaus PORS, Bradford (GB); Goreti Ribeiro MORAIS, Bradford (GB)

(21) Appl. No.: 19/147,461

(22) PCT Filed: Jan. 17, 2024
(86) PCT No.: PCT/EP2024/051080

§ 371 Date: Jul. 11, 2025

(30) Foreign Application Priority Data
Jan. 17, 2023 (GB) 2300668.7

(51) Int. Cl. C07D 401/14; A61K 31/404; A61K 31/4439; A61K 47/68; A61P 35/00; C07D 209/60; C07D 405/06; C07D 409/06; C07D 487/04

(52) U.S. Cl. CPC C07D 401/14; A61K 31/404; A61K 31/4439; A61K 47/68; A61P 35/00; C07D 209/60; C07D 405/06; C07D 409/06; C07D 487/04

(57) ABSTRACT

The present invention relates to a group of compounds that generally fall within the class of compounds known as the duocarmycins. The present invention provides a novel group of duocarmycins with a structure according to Formula (I). The compounds have efficacy in the treatment of cancer.

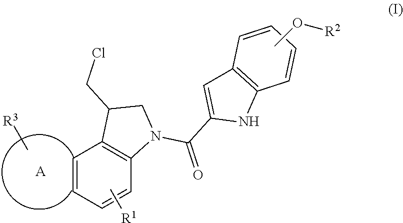

This invention relates to a group of compounds that generally fall within the class of compounds known as the duocarmycins. The present invention relates to the compounds and to the use of the compounds in the treatment of cancer. The present invention also contemplates methods of treatment with the compounds of the present invention.

### BACKGROUND

The duocarmycins belong to a class of agent that have fascinated scientists for over 4 decades. Their exquisite potency, unique mechanism of action and efficacy in multi-drug resistant tumour models attracts them to medicinal chemists and drug hunters. However, despite great advances in fine-tuning biological activity through structural-activity relationship studies, their therapeutic potential has yet to be unlocked for cancer therapy.

Duocarmycins are a family of DNA minor groove binding compounds originally identified in Streptomyces with exquisite cytotoxicity. Cyclopropapyrroloindole-based duocarmycins such as (+)-duocarmycin SA (DSA) and CC-1065 consist of a DNA recognition motif (DNA-RM) and a pharmacophore responsible for alkylating DNA. The DNA-RM initially binds in the minor groove in a non-covalent manner, which induces a conformation change and aligns the cyclopropane of the alkylation subunit for nucleophilic attack by the N3-position of adenine in A-T rich regions. Synthetic manipulation of the DNA alkylating subunit dramatically impacts on the cellular potency in the pM-nM range with the majority of compounds forcing cells to undergo apoptosis via direct S-phase inhibition and subsequent cell cycle arrest.

Seco-duocarmycins derived from common scaffolds such as CI, DSA, CPI and CBI are precursor molecules that contain a phenolic hydroxyl group that is responsible for initiating rearrangement of the pharmacophore to produce a cyclopropane-containing cytotoxin via a process known as spirocyclisation.

Unfortunately, despite their considerable potential as therapeutics, market approval for the clinical use of duocarmycins has not yet been granted. Clinical administration of adozelesin, bizelesin and two carbamate prodrugs carzelesin and KW-2189 has been associated with severe side effects and a narrow therapeutic index, leading to all trials being discontinued. Despite the clinical failure, the interest in these molecules has remained high and led to a large body of work which has demonstrated fascinating insights in both biological and chemical explorations. The inherent capacity of the duocarmycins to evade traditional resistance mechanisms and retain exquisite potency in multi-drug resistant cells still attracts considerable interest from both academia and industry in order to develop new approaches for this class of molecule with a focus on expanding the therapeutic index for patient benefit.

Several methodologies have been investigated to assist in the improvement of the therapeutic index of the duocarmycins. Amongst these there have been investigated: hypoxia-activated duocarmycin prodrugs; cytochrome P450 oxidation prodrugs; phenol masking prodrugs; antibody-drug conjugates and small molecule drug conjugate. Each will be discussed in some detail below.

### Hypoxia-Activated Duocarmycin Prodrugs

Solid tumours often contain regions of high cell density and poor vascularisation leading to low oxygenation and hypoxia. These regions are often therapy resistant and house some of the most aggressive cancerous cells. While hypoxic fractions remain an obstacle to effective treatment, they also offer opportunities for the development of hypoxia-activated prodrugs (HAPs). Generally, HAPs are designed to exploit the presence of one- and two-electron oxidoreductases which can catalyse the reduction of a trigger group critical to prodrug activation and hypoxia selectivity. Early proof-of-concept (PoC) studies using seco-amino CI-based duocarmycins provided evidence these possessed significant biological potency and hence were suitable for prodrug derivatisation. Through a number of interesting mechanistic studies the hypoxic cytotoxicity ratios (HCR, IC₅₀(oxic)/IC₅₀(hypoxic)) were improved, ultimately leading to the development of a sulphonamide-containing CBI analogues suitable as HAPs. To increase aqueous solubility for in vivo investigations, phosphate preprodrugs were developed. Significant potentiation was observed between these two compounds and radiotherapy or chemotherapy (gemcitabine and docetaxel) in mice xenograft models, with full remission seen in some mice. However, maximum tolerated doses could not be calculated due to sporadic acute toxicity. While there are a number of NO₂-containing, hypoxia-activated duocarmycin drugs on the market, spontaneous bioreduction of the NO₂ group might lead to off-target effects. Hence, there is a need for compounds that avoid sporadic acute toxicity events and/or avoid the potential for off target events.

The Boger lab has described N-acyl O-amino phenol HAPs that are sensitive to cleavage at the weak O—N bond, but which were reported to be preferentially activated under hypoxic conditions. While early-staged compounds suffered from poor in vivo stability, cyclic counterparts were shown to possess greater stability and yet still rapidly cleaved under in vitro reductive conditions.

Although eight HAPs have progressed to clinical evaluation none have been approved. Any future successes of duocarmycins-based HAPs will likely be linked to the design of better (i) drug candidates that enables a balance between retaining potency and increasing the bystander effect in the tumour microenvironment (TME) and (ii) clinical trials that involve patients harbouring tumours that are severely hypoxic as validated via a reliable biomarker approach.

### Cytochrome P450 Oxidation

Cytochrome P450 (CYP) enzymes are a superfamily of mixed function oxidases of which CYP1-3 subfamily members are unique in their ability to oxidise drugs. Their catalytic activity and substrate specificity has been explored to see whether they can be pursued in drug design. Given several isoforms including CYP1A1, 1B1, 2S1 and 2W1 are overexpressed in many human tumour types compared to normal tissue, an opportunity to design drugs whose activity is dependent on a critical functional group that can be unmasked or restored by CYP oxidation selectively in tumour tissue. In this context, the seco-duocarmycins lend themselves as candidate molecules due to the importance of the phenolic OH group in the central chloromethylindoline trigger unit and removal of the OH group in CBI and CPI scaffolds leads to more than a 1000-fold loss in potency. In PoC studies it has been demonstrated that both CYP1A1 and CYP2W1 are capable of restoring the phenolic OH group critical for biological activity. Lead bioprecursors have been investigated in vivo and shown to significantly delay tumour growth in CYP1A1 and CYP2W1-expressing xenograft models. Importantly, no body weight loss was observed, indicating such reengineered duocarmycins bioprecursors can be safely administered while retaining anticancer activity.

However, such duocarmycin prodrugs can only be applied to tumours where the required enzymes are overexpressed. Thus, there is a need for a more general approach to the provision of duocarmycin drug analogues which are tolerated by the patient.

### Phenol Masking Prodrugs

The phenol masking prodrug is a collection of approaches sharing the common feature of masking the phenol (or primary amine) group with a cleavable moiety. Carbamate duocarmycins are an example of masking at the phenol, but non-specific prodrug activation contributed to off-target toxicity.

Two recent studies have investigated seco-CBIs as photoactivable therapeutics by chemical inactivation of the phenolic OH to feature an O-linked 2-nitropiperonyl UV-labile group. In both cases the studies demonstrated efficient in vitro activation after exposure to UV light at 365 nm. A drawback of this therapeutic approach is that light has to be directed to a known tumour mass, which at present limits its use in metastatic cancers. However, photoactivatable chemotherapeutics could be useful in unresectable tumours or where removal of tumour mass is incomplete. Thus, there is a need for duocarmycins that can be activated without the need to expose them to light.

There have been attempts to lock duocarmycins in their seco form by attaching peptides to the functional primary amine groups with the activation reliant on peptide cleavage by tumour associated peptidases. This was, for example, explored by Twum et al, where an aminoCBI was primed for conjugation through the amine to a peptide unit designed to be cleaved by Prostate Specific Antigen (PSA). The study focused on establishing a stringent chemical synthesis pathway for generation of the appropriate seco-NH₂—CBI warhead while in vitro cytotoxicity of the aminoCBI in PSA expressing cell lines was confirmed.

Extensive work has been carried out on the development of glycosidic-modified duocarmycins designed to be suitable for use in antibody-directed enzyme prodrug therapy (ADEPT) technology. In this system, the phenolic OH group in the DNA alkylating subunit is chemically modified via an O-linked glycosidic group leading to inactivation through an inability to spirocyclise. Cellular potency was shown to more than a 1000-fold higher in A549 cells that expressed the presence of the target glycosidic enzyme.

A study by Tietze, Roffler and colleagues also employed a gene-directed enzyme prodrug therapy (GDEPT) approach. To demonstrate the potential of this approach, intratumoural injection of adenovirus expressing β-glucuronidase fused to an optimised transmembrane domain were administered to human CL1-5 xenografts in mice alongside a gene-directed enzyme prodrug. Survival rates were extended from 35 days with no treatment to over 150 days for mice treated with the prodrug and vector, with seven out of nine mice reaching full remission. This experiment represented a PoC in terms of enzyme delivery using a non-antibody approach and indicated the potential in using delivery options such as bacterial expression systems and nanoparticle delivery technology.

Both irradiation and chemotherapeutic treatment have in some cases been reported to result in senescent cell formation, which is effective at inhibiting tumour growth, but may also function as a means of escaping apoptosis and allowing a possible reversal out of senescence. Interestingly, galactose-protected bifunctional CBI-dimers have recently been reported as potential senolytics on the basis that β-galactosidase is more prevalent in senescent cells. While a senolytic function was demonstrated in vitro and in vivo, no toxicity data were shown in this study. However, given the extreme toxicity of duocarmycins and low expression levels of β galactosidase in all tissues, systemic toxicity might be anticipated and hence would need to be carefully managed in the clinic.

### Antibody-Drug Conjugates

Antibody-Drug Conjugates (ADCs) have gained prominence in the last decade with 12 approved drugs and over a hundred in clinical trials. ADCs combine the excellent targeting properties of antibodies with the cell-killing power of conjugated payloads.

Human epidermal growth factor receptor 2 (HER2) is the target of both the clinically approved ADC trastuzumab emtansine (Kadcyla, T-DM1) and a duocarmycin ADC (SYD985) which is currently in Phase III clinical trials for breast and gastric cancers. SYD985 uses a duocarmycin derived payload (duocarmycin-hydroxybenzamide-azaindole, DUBA). The DUBA is O-linked to a self-elimination group designed to undergo spontaneous breakdown after cleavage at an adjacent valine cittruline (VC) site by cathepsin B. This elimination releases a seco-DUBA which undergoes spirocyclisation to an active form. The payload is conjugated to the antibody through a thiol reactive maleimide to cysteines exposed through disulphide bond reduction. The drug to antibody ratio (DAR) can be partially controlled by adjusting the amount of reducing agent. The significance of the DAR is highlighted by SYD983, a forerunner of SYD985, which has a wide range of DARs from 2 to 8. Hydrophobic interaction chromatography (HIC) was used to separate the mixture and the resultant SYD985, with an average DAR of 2.7, is twice as effective at killing HER2-expressing SK-BR-3 breast cancer cells than SYD983.

In a recent study, it was demonstrated that SYD985 is effective against patient-derived xenografts (PDXs) with resistance to T-DM1. This may be explained by the SYD985 payload not requiring lysosomal degradation to be released, low levels of HER2 being sufficient for cytotoxicity and drug efflux pumps having minimal impact. SYD985 was found to be 3 to 50 times more effective in cell lines expressing low levels of HER2 compared to T-DM1, whereas the results were similar when HER2 was expressed at high levels.

MGC018 is an ADC which uses a DUBA payload identical to that in SYD985 conjugated to a mAb which targets B7-H3, an immune check point antigen which is highly expressed in most solid tumours. MGC018 recently entered phase 1/11 clinical trials for patients with solid tumours as a monotherapy or combination with MGA012, a PD-1 targeting immune check point inhibitor. Preliminary safety reporting from the trial suggests that side effects are mostly tolerable and demonstrate early sign of efficacy in some patients.

BMS-936561 (MDX-1203), a CD70 targeting ADC incorporating a duocarmycin as a warhead, has also previously reached clinical trials. BMS-936561 is conjugated through a linker with a maleimide and VC cleavage site attached to the DNA-RM of the duocarmycin. The phenol is protected by a carbamate group which can be removed by endogenous carboxylesterases. However, despite a completion of a phase I clinical trial targeting renal cell cancer and non-Hodgkin's lymphoma with well tolerated doses identified, the programme has been discontinued.

A recent study reported on the anti-EGFR bispecific antibody complexed with a tetra-duocarmycin cotinine compound. Similarly, to SYD985, the duocarmycin payload was conjugated via an equivalent O-linked VC self-elimination linker but produced with a homogenous DAR. The conjugate was produced by linking two cotinine modules with the peptide linker GSKGSKGSKGSKK and then by tethering the chemotoxin to each of the 4 first lysines. Subsequently, this part of the conjugate was complexed with the cotinine specific single chain variable fragment (scVf) of the bispecific antibody. This ADC elicited significant tumour growth inhibition in murine lung adenocarcinoma models, with no detectable weight loss during the 5 week treatment period.

In addition, Bondis are progressing 4 duocarmycin based ADCs.

As referenced in the photoactivatable subsection, a near-infrared photo-controllable theranostic duocarmycin antibody-conjugate has been reported. The CBI-based duocarmycin is O-linked to a cyanine caging group by N,N′-diethylethylendiamine at the C4′ position, which is then conjugated to panitumumab (anti-EGFR antibody) through a N-hydroxysuccinimide by non-specific deamidation of asparagine and glutamine. The payload is released through exposure to light at 780 nm. Additionally, the conjugate can be visualised by external illumination, which was used to determine maximum tumour accumulation and hence the optimal time for activation. The conjugate demonstrated in vitro pM cytotoxicity after activation using external light exposure. Reaccumulation was also monitored, facilitating an informed second dose of activating light. A benefit of this tool is that haemoglobin and water have low absorbance in the 600-850 nm range, which means noise is reduced, enabling moderately deeper tissue imaging.

Phosphate groups have also been explored as hydroxyl protecting and water solubilising groups for use in ADCs. Generally, excellent differential cytotoxicity in the presence and absence of phosphatases have been observed, demonstrating the suitability of phosphates in ADC design.

### Small Molecule Drug Conjugate

Small molecule drug conjugates (SMDC) are designed to exploit the specificities of protein-targeting small molecules and the cytotoxicity of non-specific chemotoxins by combining them with a cleavable linker.

Initial work published in 2014 demonstrated that duocarmycins could be viable payloads for SMDCs by combining inhibitors for Carbonic Anhydrase IX (CA9), which is over expressed in hypoxic cancer cells, with a seco-duocarmycin O-linked to a self-eliminating group. However, the conjugate only demonstrated modest tumour growth inhibition after 7 days of treatment in mice with SKRC52 renal cell carcinoma grown subcutaneously.

More recently, a PoC study which combined a glutamate carboxylpeptidase II (GCPII) targeting molecule (DUPA) and a DSA DNA alkylation subunit derived from duocarmycin with a VC cleavable linker (DUPA-DSA) was reported. GCPII is a cell surface protein that undergoes endocytosis when bound to a ligand and is reportedly 1000-fold more prevalent in cancerous cells and DUPA-DSA has been demonstrated to be significantly potent in GCPII expressing cells in vitro.

Despite the promise of the powerful cell-killing duocarmycins they have yet to be approved for clinical use. Several promising tumour-selective prodrug and ADC approaches have been demonstrated; however, they have experienced relatively little success.

Therefore, there remains a need for a duocarmycin analogue that can provide an efficacious cancer therapy whilst avoiding unwanted adverse effects. For both prodrug and ADC strategies low enzyme or antigen expression as well as tumour penetration are possible obstacles to effective treatment. Furthermore, the exquisite potency of the duocarmycins might prevent a significant bystander effect and reduced drug concentration, which is likely to contribute to distinct subpopulations of cells surviving. Not because of resistance but because of inadequate drug delivery. Complete eradication of the tumour infrastructure necessitates further refinement of duocarmycins-based therapies.

### BRIEF SUMMARY OF THE DISCLOSURE

In accordance with the present invention there is provided a compound of formula I, or a pharmaceutically acceptable salt thereof:

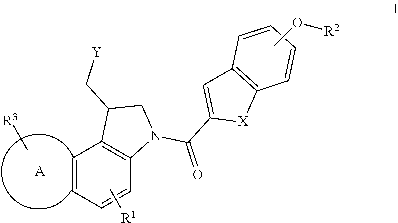

wherein:
A represents a 5 or 6 membered ring wherein there are 3 or 4 atoms not represented in the ring and the 3 or 4 atoms are selected as follows: 0, 1 or 2 of the atoms are selected independently from C═O, N, NH, S, or O with the balance of the atoms being made up of CH;
X represents NH, S or O;
Y represents a halo group;
R¹ represents a group selected from: —OR^{4a}, —NR^{4a}R^{4b}, substituted or unsubstituted C₁₋₆ alkyl, substituted or unsubstituted C₅₋₁₀ cycloalkyl, substituted or unsubstituted C₅₋₁₀ heterocycloalkyl, substituted or unsubstituted C₆₋₁₀ aryl, and substituted or unsubstituted C₅₋₁₀ heteroaryl,
   wherein, when substituted, the R¹ group is substituted with 1, 2, or 3 groups independently selected from: halo, —OR^{4a}, —NR^{4a}R^{4b}, —NO₂, C₁₋₆ alkyl, C₁₋₆ haloalkyl, C₁₋₆ alkyl substituted with OH, C₁₋₆ alkyl substituted with OMe, and C₁₋₆ alkyl substituted with ═O;
R² represents a group selected from: H, C₁₋₆ alkyl, C₂₋₆ alkenyl, C₂₋₆ alkynyl, and —C(O)—R⁵;
R³ represents a group selected from: H, —OH, halo, C₁₋₆ alkyl, C₁₋₆ haloalkyl, C₁₋₆ alkoxy, —NR^{6a}R^{6b}, C₁₋₆ alkoxy substituted with C₆₋₁₀ aryl and C₁₋₆ alkyl substituted with C₆₋₁₀ aryl;
R^{4a} and R^{4b} are each independently selected from: C₁₋₆ alkyl, C₂₋₆ alkenyl, C₂₋₆ alkynyl, C₁₋₆ alkyl substituted with OH, C₁₋₆ alkyl substituted with OMe, C₁₋₆ alkyl substituted with ═O, C₁₋₆ alkyl substituted with a substituted or unsubstituted C₆₋₁₀ aryl, —C(O)—C₁₋₆alkyl, —(CR^{7a}R^{7b})ₙC(O)OR⁸, —(CR^{7a}R^{7b})ₙC(O)NHR⁸, substituted or unsubstituted C₅₋₁₀ cycloalkyl, substituted or unsubstituted C₅₋₁₀ heterocycloalkyl, substituted or unsubstituted C₆₋₁₀ aryl, and substituted or unsubstituted C₅₋₁₀ heteroaryl;
   or R^{4b} is H;
   wherein, when substituted, the R^{4a} and R^{4b} group is substituted with 1 or 2 groups selected from: halo, OH, —NO₂, C₁₋₆ alkyl, C₁₋₆ alkoxy, C₁₋₆ haloalkyl, C₁₋₆ alkyl substituted with OH, C₁₋₆ alkyl substituted with OMe, C₁₋₆ alkyl substituted with ═O, and —NH₂;
n is 1, 2, or 3 (preferably 1);
R⁵ represents a group selected from: C₁₋₆ alkyl, C₂₋₆ alkenyl, and C₂₋₆ alkynyl;
R^{6a} and R^{6b} are each independently selected from: H and C₁₋₆ alkyl;
one of R^{7a} and R^{7b} is a group selected from: H, C₁₋₆ alkyl and C₁₋₆ alkyl substituted with —OH, —SH, —SMe, —NH₂, —C(O)OH, —C(O)NH₂, guanidine, C₆₋₁₀ aryl, C₆₋₁₀ aryl substituted with —OH, or C₅₋₁₀ heteroaryl; and all other R^{7a} and R^{7b} are H; and
R⁸ represents a group selected from: H and C₁₋₆ alkyl;
provided that R¹ is not —OBn when R² is methyl.

In accordance with the present invention there is provided a compound of formula I, or a pharmaceutically acceptable salt thereof:

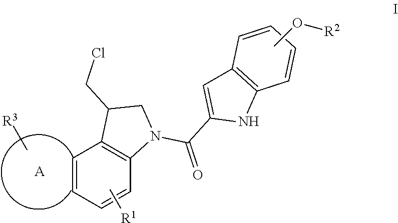

wherein:
A represents a 5 or 6 membered ring wherein there are 3 or 4 atoms not represented in the ring and the 3 or 4 atoms are selected as follows: 0, 1 or 2 of the atoms are selected independently from C═O, N, NH, or O with the balance of the atoms being made up of CH;
R¹ represents a group selected from: —OR^{4a}, —NR^{4a}R^{4b}, substituted or unsubstituted C₁₋₆ alkyl, substituted or unsubstituted C₅₋₁₀ cycloalkyl, substituted or unsubstituted C₅₋₁₀ heterocycloalkyl, substituted or unsubstituted C₆₋₁₀ aryl, and substituted or unsubstituted C₅₋₁₀ heteroaryl,
   wherein, when substituted, the R¹ group is substituted with 1, 2, or 3 groups independently selected from: halo, —OR^{4a}, —NR^{4a}R^{4b}, C₁₋₆ alkyl, C₁₋₆ haloalkyl, C₁₋₆ alkyl substituted with OH, C₁₋₆ alkyl substituted with OMe, and C₁₋₆ alkyl substituted with ═O;
R² represents a group selected from: H, C₁₋₆ alkyl, C₂₋₆ alkenyl, C₂₋₆ alkynyl, and —C(O)—R⁵;
R³ represents a group selected from: H, —OH, halo, C₁₋₆ haloalkyl, C₁₋₆ alkoxy, —NR^{6a}R^{6b}, C₁₋₆ alkoxy substituted with C₆₋₁₀ aryl and C₁₋₆ alkyl substituted with C₆₋₁₀ aryl;
R^{4a} and R^{4b} are each independently selected from: H, C₁₋₆ alkyl, C₂₋₆ alkenyl, C₂₋₆ alkynyl, C₁₋₆ alkyl substituted with OH, C₁₋₆ alkyl substituted with OMe, C₁₋₆ alkyl substituted with ═O, C₁₋₆ alkyl substituted with C₆₋₁₀ aryl, —C(O)—C₁₋₆alkyl, —(CR^{7a}R^{7b})ₙC(O)OR⁸, —(CR^{7a}R^{7b})ₙC(O)NHR⁸, substituted or unsubstituted C₅₋₁₀ cycloalkyl, substituted or unsubstituted C₅₋₁₀ heterocycloalkyl, substituted or unsubstituted C₆₋₁₀ aryl, and substituted or unsubstituted C₅₋₁₀ heteroaryl;
   wherein, when substituted, the R^{4a} and R^{4b} group is substituted with 1 or 2 groups selected from: halo, OH, —NO₂, C₁₋₆ alkyl, C₁₋₆ alkoxy, C₁₋₆ haloalkyl, C₁₋₆ alkyl substituted with OH, C₁₋₆ alkyl substituted with OMe, C₁₋₆ alkyl substituted with ═O, and —NH₂;
n is 1, 2, or 3 (preferably 1);
R⁵ represents a group selected from: C₁₋₆ alkyl, C₂₋₆ alkenyl, and C₂₋₆ alkynyl;
R^{6a} and R^{6b} are each independently selected from: H and C₁₋₆ alkyl;
one of R^{7a} and R^{7b} is a group selected from: H, C₁₋₆ alkyl and C₁₋₆ alkyl substituted with —OH, —SH, —SMe, —NH₂, —C(O)OH, —C(O)NH₂, guanidine, C₆₋₁₀ aryl, C₆₋₁₀ aryl substituted with —OH, or C₅₋₁₀ heteroaryl; and all other R^{7a} and R^{7b} are H; and
R⁸ represents a group selected from: H and C₁₋₆ alkyl;
provided that R¹ is not OH when R² is methyl.

In certain embodiments, A represents a 5 or 6 membered ring wherein there are 3 or 4 atoms not represented in the ring and the 3 or 4 atoms are selected as follows: 0, 1 or 2 of the atoms are selected independently from C═O, N, NH, or O with the balance of the atoms being made up of CH.

In embodiments X is NH.

In embodiments Y is chloro or bromo.

For the avoidance of doubt the ring represented by A totals 5 or 6 atoms including the two carbon atoms that form the termini of the circular line representing A. Thus, the 3 or 4 atoms of A plus the two carbon atoms at the terminus result in a 5 or 6 membered ring. The 5 or 6 membered ring represented by A may be aromatic.

A may represent one of the following rings: benzene, pyridine, pyridazine, pyrimidine, pyrazine, pyrrole, pyrazole, furan, pyrrolinone, imidazole, or oxazole.

A may represent one of the following rings: thiophene, benzene, pyridine, pyridazine, pyrimidine, pyrazine, pyrrole, pyrazole, furan, pyrrolinone, imidazole, or oxazole.

In embodiments the compound of formula I is a compound according to one of the following Formula:

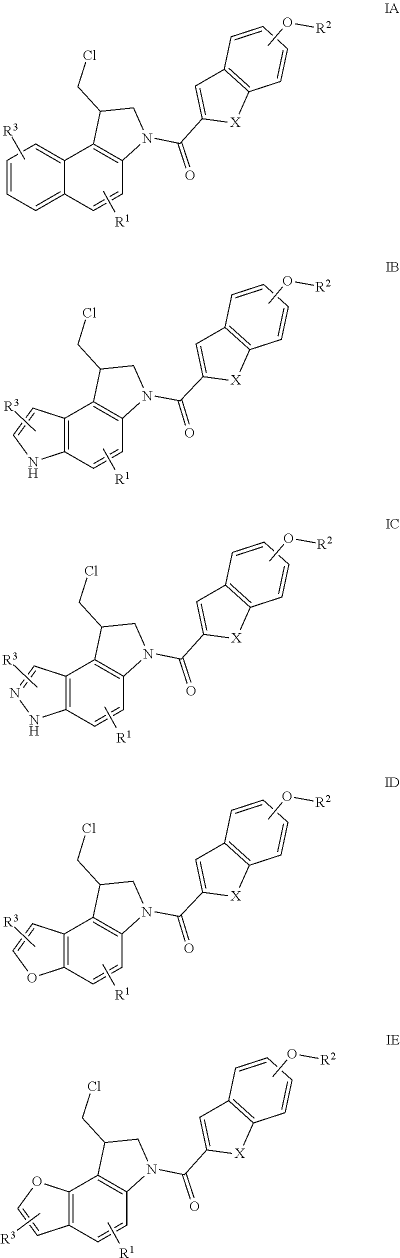

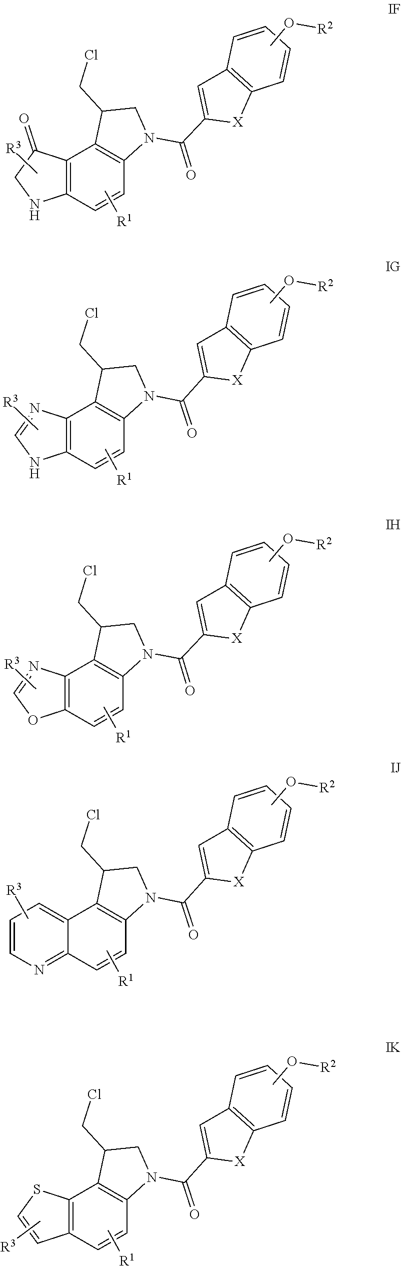

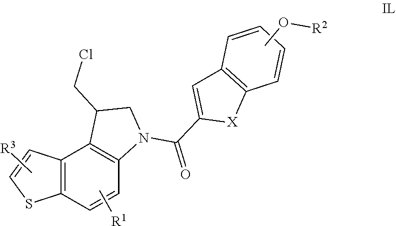

In certain embodiments, for the compounds of Formula IA to IL, X is NH.

Preferably the compound of the present invention is a compound of Formula IA.

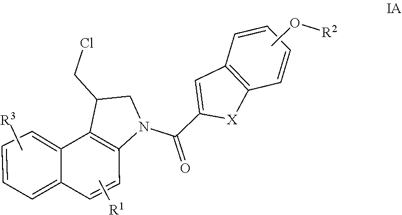

Preferably the compound of the present invention is a compound of Formula IA′.

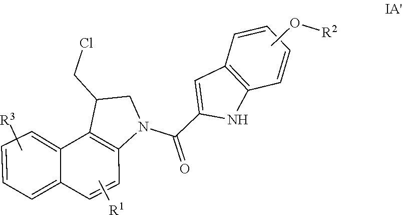

In embodiments of the present invention the —OR² group may be substituted on any suitable carbon atom of the indole ring into which the bond enters by replacing a hydrogen atom that would ordinarily be substituted on the carbon atom, including the nitrogen containing ring. In embodiments, the compound of formula I is a compound according to formula IAa:

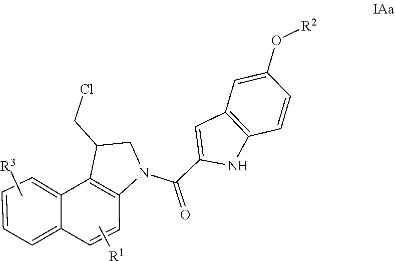

The R¹ and R³ groups may be substituted on a carbon atom of the ring into which their bond enters by replacing a hydrogen atom that would ordinarily be substituted on the carbon atom. By way of a representative example for compounds of formula (I) we have represented potential substitution locations on the ring of compounds of formula IA and IB. R¹ may be substituted at position a or b indicated below. R³ may be substituted at position c, d, e, or f. In certain embodiments, R¹ is substituted at position b. In certain embodiments, R³ is substituted at position f.

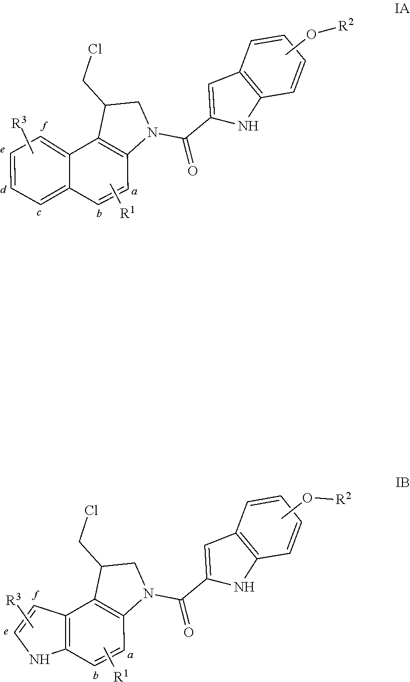

In embodiments the compound of formula I is a compound according to formula IIA:

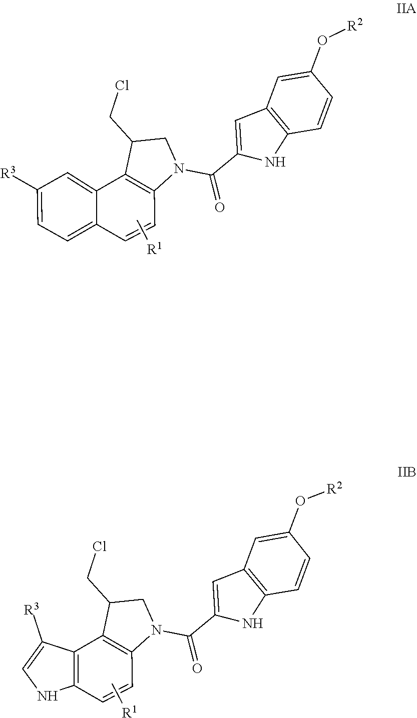

In certain embodiments, R³ represents a group selected from: H, —OH, halo, C₁₋₆ haloalkyl, C₁₋₆ alkoxy, —NR^{6a}R^{6b}, and C₁₋₆ alkoxy substituted with C₆₋₁₀ aryl.

In certain embodiments, R³ represents a group selected from: H, —OH, C₁₋₆ alkyl, C₁₋₆ alkoxy, —NR^{6a}R^{6b}, and C₁₋₆ alkoxy substituted with C₆₋₁₀ aryl.

In certain embodiments, R^{6a} and R^{6b} are each independently selected from: H, methyl, and ethyl.

In certain embodiments R³ represents a group selected from: H, —OH, methyl, ethyl, —OMe, —NH₂, —NHMe, —N(Me)₂, and —OBn. Preferably, R³ is H or methyl.

Thus, in embodiments, the compound of formula I is a compound according to formula IIa:

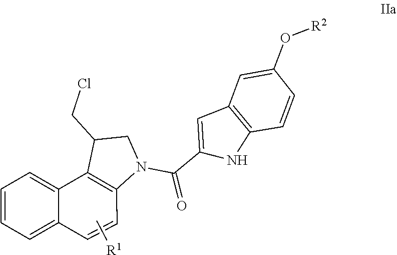

In certain embodiments R² represents a group selected from: C₁₋₆ alkyl and —C(O)—R⁵.

In certain embodiments R² represents a group selected from: H, methyl, ethyl, propyl, butyl, and —C(O)—R⁵. Preferably, R² is methyl or —C(O)—R⁵.

R⁵ may be C₁₋₆ alkyl or C₂₋₆ alkynyl. In certain embodiments R⁵ is methyl or pentynyl.

Accordingly, in certain embodiments R² is methyl, —C(O)-methyl, or —C(O)-pentynyl (optionally wherein the pentynyl has a terminal triple bond). Thus, R² may be methyl, —C(O)-methyl, or

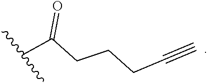 [SMILES: *C(=O)CCCC#C]

Accordingly, compounds of the invention may be a compound according to formulae IIIa or IIIb:

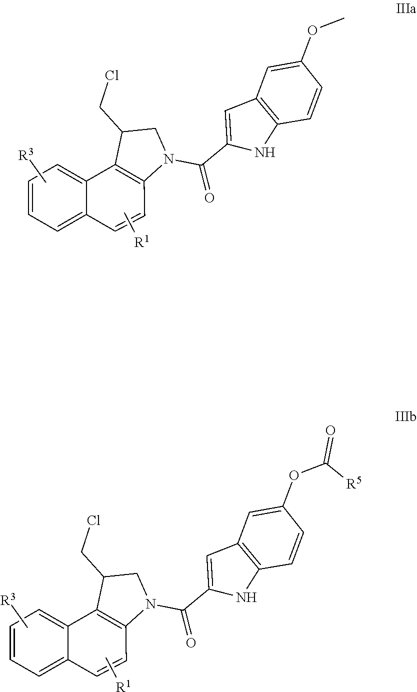

In certain embodiments, compounds of the present invention are compounds according to formulae IVa or IVb:

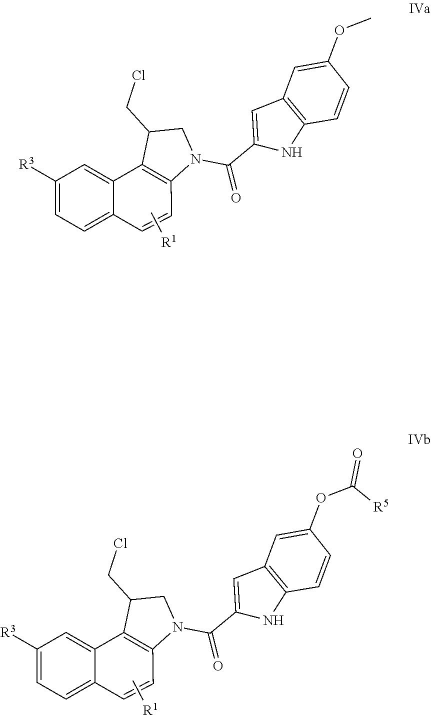

As mentioned above, in embodiments R³ is preferably H. Therefore, the compounds of the present invention may be a compound according to the formulae Va or Vb:

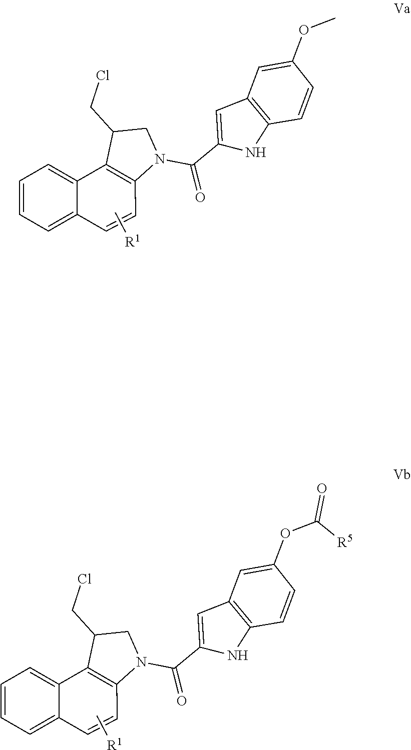

In certain embodiments the compounds of the present invention may be compounds according to formula VI:

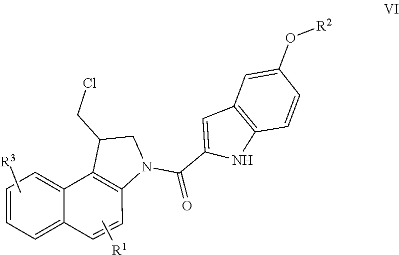

In certain embodiments the compounds of the present invention may be compounds according to formulae VIIa or VIIb:

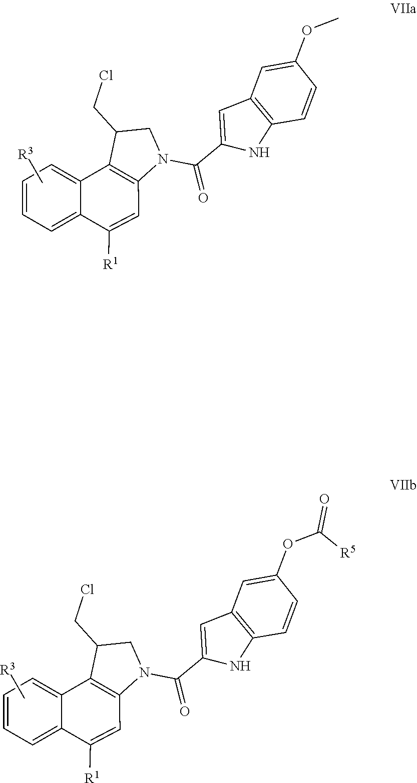

In certain embodiments the compounds of the present invention may be compounds according to formulae VIIIa or VIIIb:

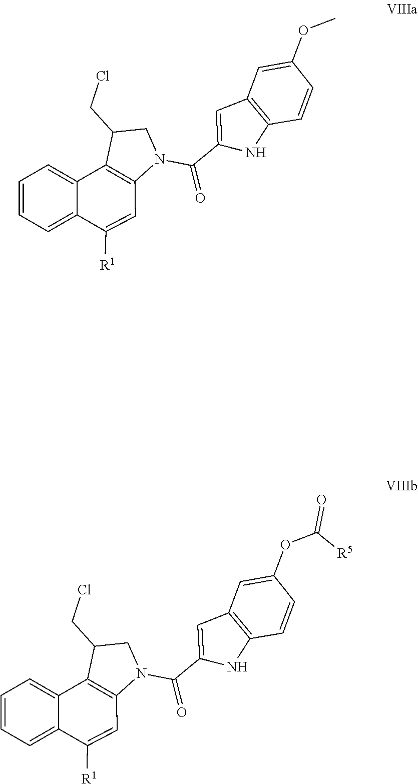

R¹ may represent a group selected from: —OR^{4a}, —NR^{4a}R^{4b}, substituted or unsubstituted C₅₋₁₀ heterocycloalkyl, substituted or unsubstituted C₆₋₁₀ aryl, and substituted or unsubstituted C₅₋₁₀ heteroaryl,
wherein, when substituted, the R¹ group is substituted with 1, 2, or 3 groups (optionally 1 or 2 groups) independently selected from: halo, OR^{4a}, —NR^{4a}R^{4b}, —NO₂, C₁₋₆ alkyl, C₁₋₆ alkyl substituted with OH, and C₁₋₆ alkyl substituted with ═O.

In embodiments R¹ is not —OBn.

R¹ may represent a group selected from: —OR^{4a}, —NR^{4a}R^{4b}, unsubstituted C₅₋₁₀ heterocycloalkyl, substituted or unsubstituted C₆₋₁₀ aryl, and substituted or unsubstituted C₅₋₁₀ heteroaryl,
wherein, when substituted, R¹ is substituted with 1 or 2, groups independently selected from: halo, OR^{4a}, —NR^{4a}R^{4b}, C₁₋₆ alkyl, C₁₋₆ alkyl substituted with OH, and C₁₋₆ alkyl substituted with ═O.

In certain embodiments, when R¹ is substituted it is substituted with 1, 2, or 3 groups (optionally 1 or 2 groups) selected from: —OH, —OMe, —CH₂OH, —CH₂OMe, —NO₂, -Me, —NH₂, —NMe₂, —OH, —F, —C(O)CH₃, and —OC(O)CH₃.

R^{4a} and R^{4b} may each be independently selected from: C₁₋₆ alkyl, C₁₋₆ alkyl substituted with OH, C₁₋₆ alkyl substituted with OMe, C₁₋₆ alkyl substituted with C₆₋₁₀ aryl, —(CR^{7a}R^{7b})ₙC(O)OR⁸, —(CR^{7a}R^{7b})ₙC(O)NHR⁸, substituted or unsubstituted C₅₋₁₀ cycloalkyl, substituted or unsubstituted C₅₋₁₀ heterocycloalkyl, and substituted or unsubstituted C₆₋₁₀ aryl;
or R^{4b} may be H;
wherein, when substituted, the R^{4a} and R^{4b} group is substituted with 1 or 2 groups selected from: halo, OH, —NO₂, C₁₋₆ alkyl, C₁₋₆ alkoxy, C₁₋₆ haloalkyl, C₁₋₆ alkyl substituted with OH, C₁₋₆ alkyl substituted with OMe, C₁₋₆ alkyl substituted with ═O, and —NH₂ (optionally halo, —NO₂, C₁₋₆ alkyl, C₁₋₆ alkoxy, and C₁₋₆ haloalkyl).

In certain embodiments R^{4a} and R^{4b} are each independently selected from: C₁₋₆ alkyl, C₁₋₆ alkyl substituted with OH, C₁₋₆ alkyl substituted with C₆₋₁₀ aryl, —(CR^{7a}R^{7b})C(O)OR⁸, —(CR^{7a}R^{7b})C(O)NHR⁸, unsubstituted C₅₋₁₀ cycloalkyl, unsubstituted C₅₋₁₀ heterocycloalkyl, and substituted or unsubstituted C₆₋₁₀ aryl
or R^{4b} may be H;
wherein, when substituted, the R^{4a} and R^{4b} group is substituted with 1 or 2 groups (optionally 1 group) selected from: halo, OH, —NO₂, C₁₋₆ alkyl, C₁₋₆ alkoxy, C₁₋₆ haloalkyl, C₁₋₆ alkyl substituted with OH, C₁₋₆ alkyl substituted with OMe, C₁₋₆ alkyl substituted with ═O, and —NH₂.

In certain embodiments R⁴ is selected from: C₁₋₆ alkyl, C₁₋₆ alkyl substituted with OH, C₁₋₄ alkyl substituted with C₆₋₁₀ aryl, —(CR^{7a}R^{7b})C(O)OR⁸, —(CR^{7a}R^{7b})C(O)NHR⁸, unsubstituted C₅₋₁₀ cycloalkyl, unsubstituted C₅₋₁₀ heterocycloalkyl, and substituted or unsubstituted C₆₋₁₀ aryl; and
R^{4b} is selected from H or C₁₋₆ alkyl;
wherein, when substituted, the R^{4a} group is substituted with 1 or 2 groups (optionally 1 group) selected from: halo, OH, —NO₂, C₁₋₆ alkyl, C₁₋₆ alkoxy, C₁₋₆ haloalkyl, C₁₋₆ alkyl substituted with OH, C₁₋₆ alkyl substituted with OMe, C₁₋₆ alkyl substituted with ═O, and —NH₂.

When substituted, the R^{4a} and R^{4b} group may be substituted with 1 or 2 groups (optionally 1 group) selected from: halo, —NO₂, C₁₋₆ alkyl, C₁₋₆ alkoxy, and C₁₋₆ haloalkyl.

In certain embodiments R^{4a} is selected from: C₁₋₆ alkyl, C₁₋₆ alkyl substituted with OH, C₁₋₄ alkyl substituted with C₆₋₁₀ aryl, —(CR^{7a}R^{7b})C(O)OR⁸, —(CR^{7a}R^{7b})C(O)NHR⁸, unsubstituted C₅₋₁₀ cycloalkyl, unsubstituted C₅₋₁₀ heterocycloalkyl, and substituted or unsubstituted C₆₋₁₀ aryl; and
R^{4b} is selected from H or C₁₋₆ alkyl;
wherein, when substituted, the R^{4a} group is substituted with 1 or 2 groups (optionally 1 group) selected from: halo, —NO₂, C₁₋₆ alkyl, C₁₋₆ alkoxy, and C₁₋₆ haloalkyl.

The R^{4a} and R^{4b} may preferably be substituted with 1 or 2 groups selected from: —OMe, —CF₃, —Cl, —NO₂, and -Me.

In certain embodiments one of R^{7a} and R^{7b} is a group selected from: H, C₁₋₆ alkyl and C₁₋₆ alkyl substituted with —OH, —SH, —SMe, —NH₂, —C(O)OH, —C(O)NH₂, guanidine, benzene, phenol, indole or imidazole, and all other R^{7a} and R^{7b} are H.

In certain embodiments n is 1 and one of R^{7a} and R^{7b} is a group selected from: C₁₋₆ alkyl and C₁₋₆ alkyl substituted with —OH, —SH, —SMe, —NH₂, —C(O)OH, —C(O)NH₂, guanidine, benzene, phenol, indole or imidazole, and the other of R^{7a} and R^{7b} is H.

In embodiments one of R^{7a} and R^{7b} is an amino acid side chain falling within the definition of all preceding definitions of R^{7a} and R^{7b}. For example, R^{7a} and R^{7b} may be —CH₂OH, —CH(OH)CH₃, —CH₂C(O)NH₂, CH₂CH₂C(O)NH₂, —CH₂SH, —CH₂CH₂SCH₃, —CH₂NH₂, —(CH₂)₄NH₂, —CH₂C(O)OH, —CH₂CH₂C(O)OH, methyl, propyl, butyl, benzyl, —CH₂-phenolyl, —CH₂-indolyl, —CH₂-imidazolyl, —CH₂CH₂CH₂-guanidinyl, and all other R^{7a} and R^{7b} are H.

In certain embodiments, R⁸ is H or Me.

In certain embodiments, R¹ is selected from: OBn (optionally provided that R² is not Me), —N(Et)₂, —NHBu, and:

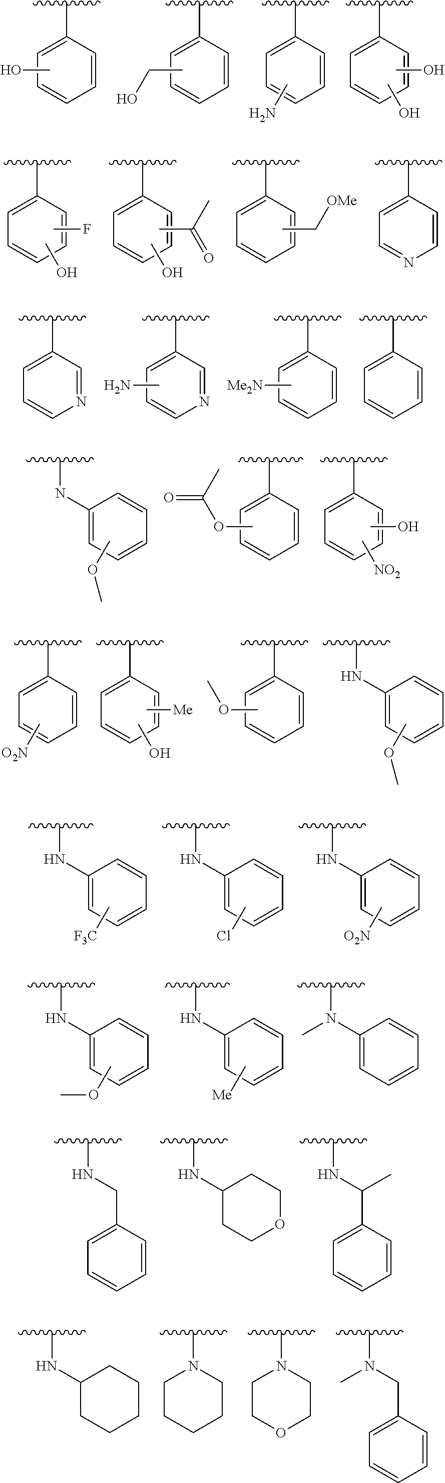

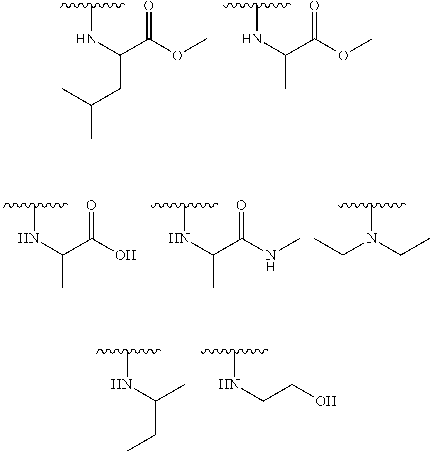

In certain embodiments, R¹ is selected from: —OH (optionally provided that R² is not H) OBn, and:

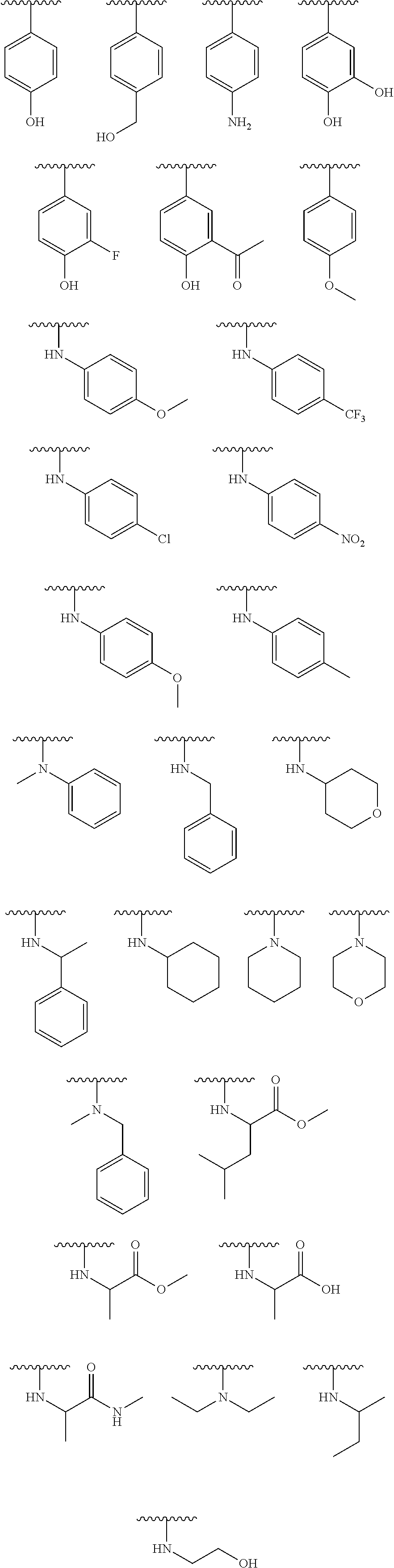

In an embodiment of the present invention the compound of Formula I is selected from:

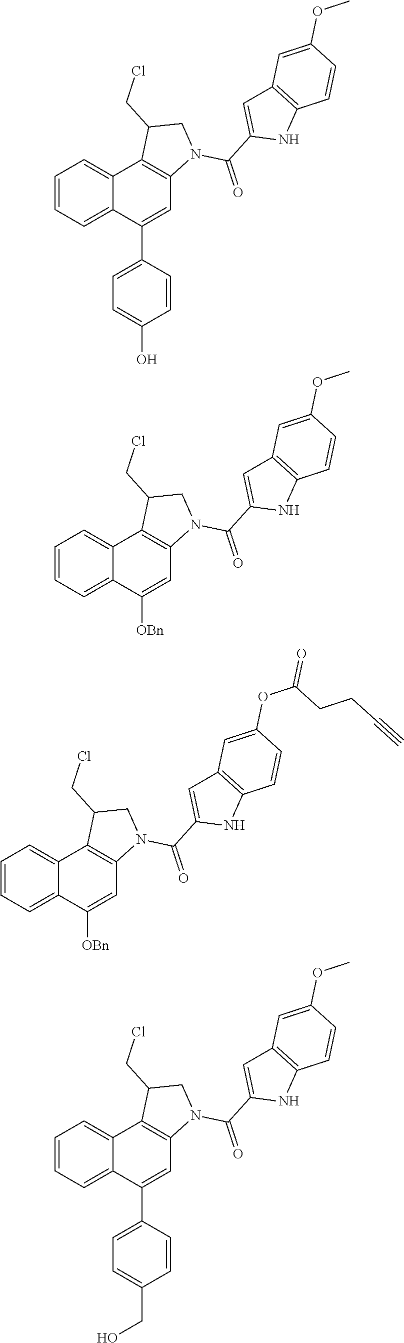 [SMILES: COc1ccc2[nH]c(C(=O)N3CC(CCl)c4c3cc(-c3ccc(O)cc3)c3ccccc43)cc2c1 ; COc1ccc2[nH]c(C(=O)N3CC(CCl)c4c3cc(OCc3ccccc3)c3ccccc43)cc2c1 ; C#CCCC(=O)Oc1ccc2[nH]c(C(=O)N3CC(CCl)c4c3cc(OCc3ccccc3)c3ccccc43)cc2c1 ; COc1ccc2[nH]c(C(=O)N3CC(CCl)c4c3cc(-c3ccc(CO)cc3)c3ccccc43)cc2c1]

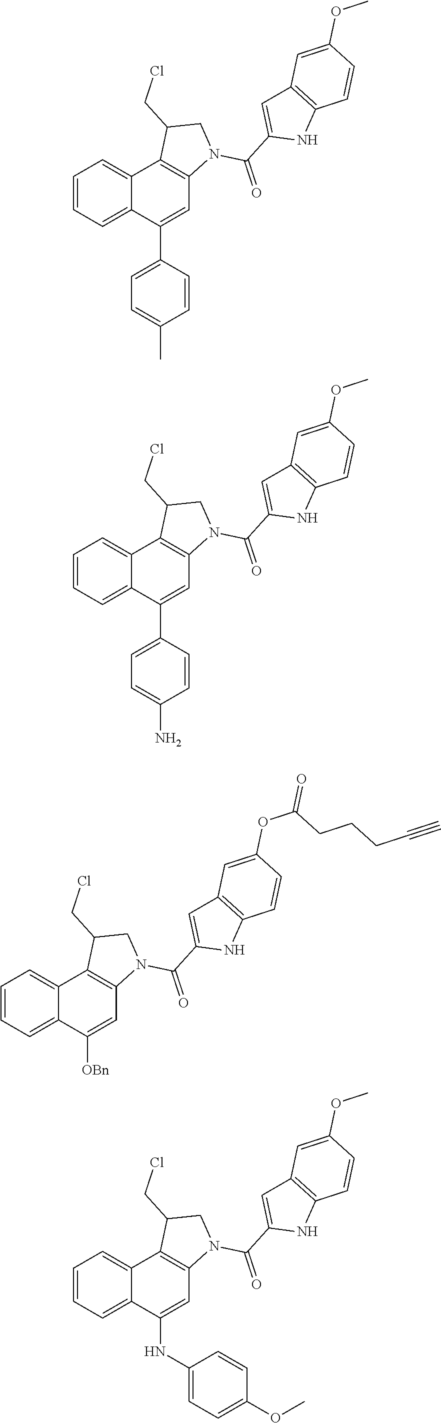 [SMILES: COc1ccc2[nH]c(C(=O)N3CC(CCl)c4c3cc(-c3ccc(C)cc3)c3ccccc43)cc2c1 ; COc1ccc2[nH]c(C(=O)N3CC(CCl)c4c3cc(-c3ccc(N)cc3)c3ccccc43)cc2c1 ; C#CCCCC(=O)Oc1ccc2[nH]c(C(=O)N3CC(CCl)c4c3cc(OCc3ccccc3)c3ccccc43)cc2c1 ; COc1ccc(Nc2cc3c(c4ccccc24)C(CCl)CN3C(=O)c2cc3cc(OC)ccc3[nH]2)cc1]

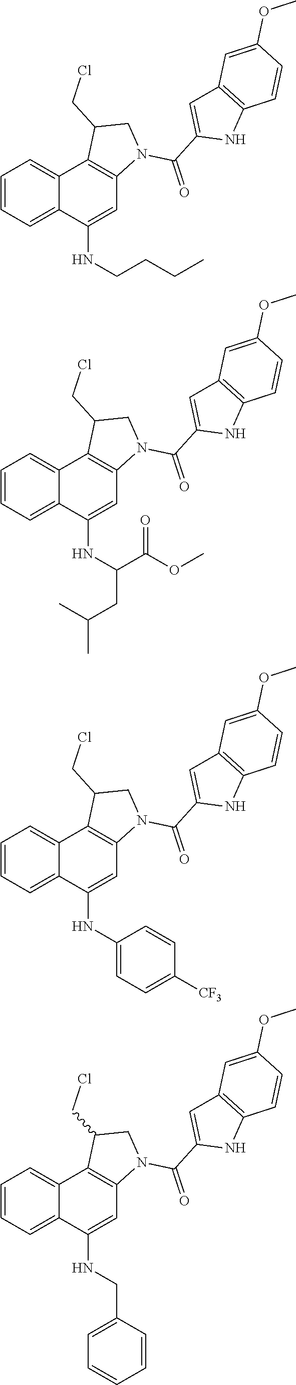 [SMILES: CCCCNc1cc2c(c3ccccc13)C(CCl)CN2C(=O)c1cc2cc(OC)ccc2[nH]1 ; COC(=O)C(CC(C)C)Nc1cc2c(c3ccccc13)C(CCl)CN2C(=O)c1cc2cc(OC)ccc2[nH]1 ; COc1ccc2[nH]c(C(=O)N3CC(CCl)c4c3cc(Nc3ccc(C(F)(F)F)cc3)c3ccccc43)cc2c1 ; COc1ccc2[nH]c(C(=O)N3CC(CCl)c4c3cc(NCc3ccccc3)c3ccccc43)cc2c1]

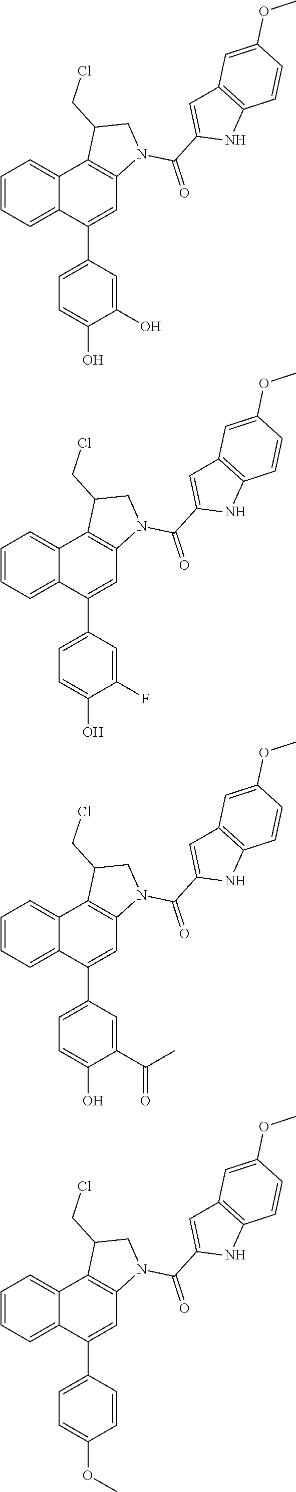 [SMILES: COc1ccc2[nH]c(C(=O)N3CC(CCl)c4c3cc(-c3ccc(O)c(O)c3)c3ccccc43)cc2c1 ; COc1ccc2[nH]c(C(=O)N3CC(CCl)c4c3cc(-c3ccc(O)c(F)c3)c3ccccc43)cc2c1 ; COc1ccc2[nH]c(C(=O)N3CC(CCl)c4c3cc(-c3ccc(O)c(C(C)=O)c3)c3ccccc43)cc2c1 ; COc1ccc(-c2cc3c(c4ccccc24)C(CCl)CN3C(=O)c2cc3cc(OC)ccc3[nH]2)cc1]

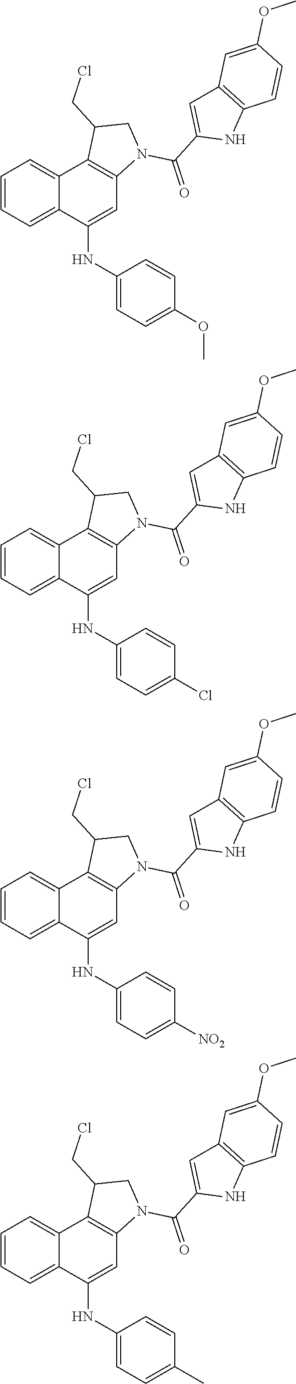 [SMILES: COc1ccc(Nc2cc3c(c4ccccc24)C(CCl)CN3C(=O)c2cc3cc(OC)ccc3[nH]2)cc1 ; COc1ccc2[nH]c(C(=O)N3CC(CCl)c4c3cc(Nc3ccc(Cl)cc3)c3ccccc43)cc2c1 ; COc1ccc2[nH]c(C(=O)N3CC(CCl)c4c3cc(Nc3ccc([N+](=O)[O-])cc3)c3ccccc43)cc2c1 ; COc1ccc2[nH]c(C(=O)N3CC(CCl)c4c3cc(Nc3ccc(C)cc3)c3ccccc43)cc2c1]

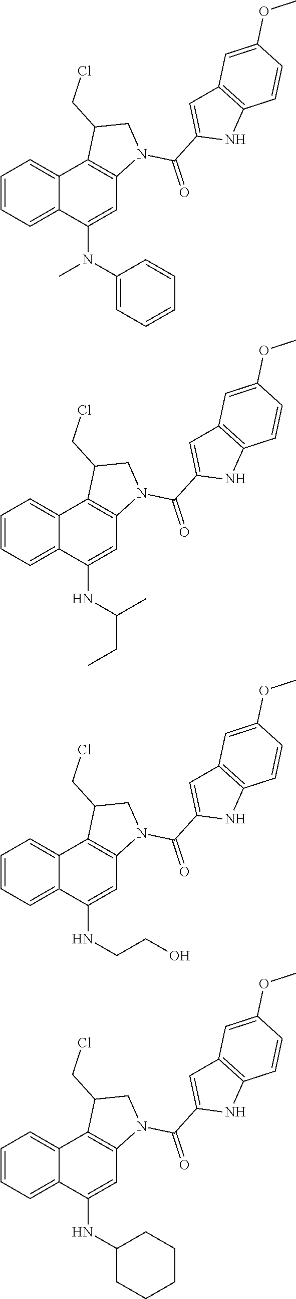 [SMILES: COc1ccc2[nH]c(C(=O)N3CC(CCl)c4c3cc(N(C)c3ccccc3)c3ccccc43)cc2c1 ; CCC(C)Nc1cc2c(c3ccccc13)C(CCl)CN2C(=O)c1cc2cc(OC)ccc2[nH]1 ; COc1ccc2[nH]c(C(=O)N3CC(CCl)c4c3cc(NCCO)c3ccccc43)cc2c1 ; COc1ccc2[nH]c(C(=O)N3CC(CCl)c4c3cc(NC3CCCCC3)c3ccccc43)cc2c1]

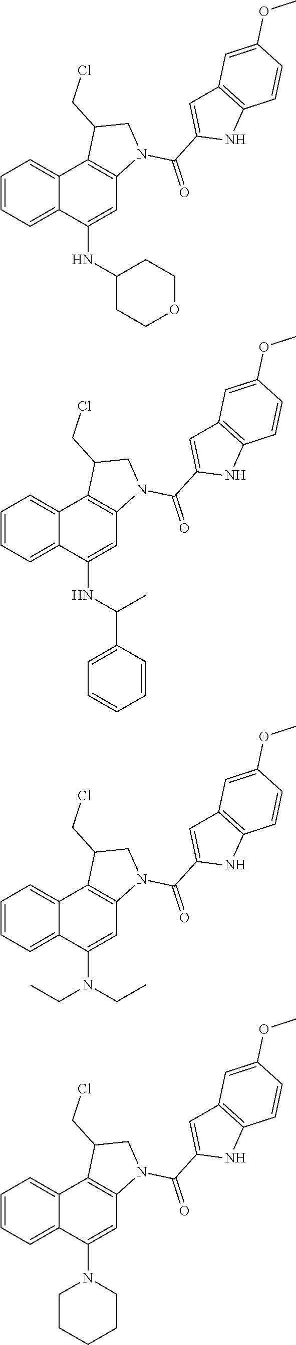 [SMILES: COc1ccc2[nH]c(C(=O)N3CC(CCl)c4c3cc(NC3CCOCC3)c3ccccc43)cc2c1 ; COc1ccc2[nH]c(C(=O)N3CC(CCl)c4c3cc(NC(C)c3ccccc3)c3ccccc43)cc2c1 ; CCN(CC)c1cc2c(c3ccccc13)C(CCl)CN2C(=O)c1cc2cc(OC)ccc2[nH]1 ; COc1ccc2[nH]c(C(=O)N3CC(CCl)c4c3cc(N3CCCCC3)c3ccccc43)cc2c1]

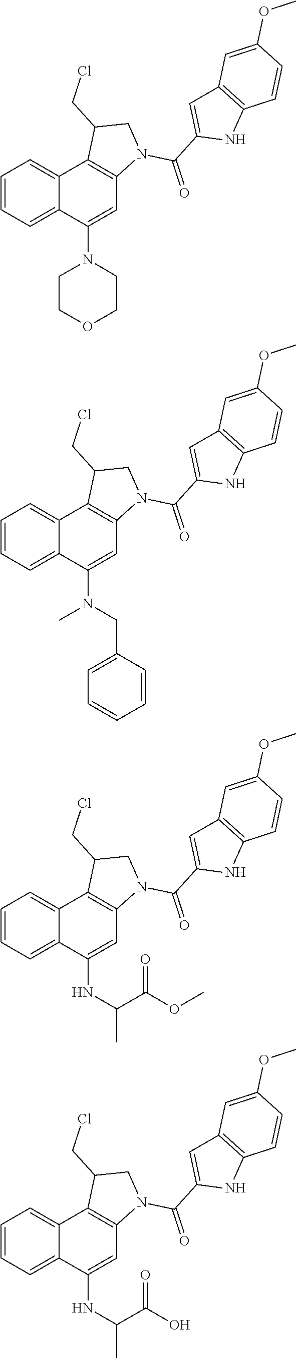 [SMILES: COc1ccc2[nH]c(C(=O)N3CC(CCl)c4c3cc(N3CCOCC3)c3ccccc43)cc2c1 ; COc1ccc2[nH]c(C(=O)N3CC(CCl)c4c3cc(N(C)Cc3ccccc3)c3ccccc43)cc2c1 ; COC(=O)C(C)Nc1cc2c(c3ccccc13)C(CCl)CN2C(=O)c1cc2cc(OC)ccc2[nH]1 ; COc1ccc2[nH]c(C(=O)N3CC(CCl)c4c3cc(NC(C)C(=O)O)c3ccccc43)cc2c1]

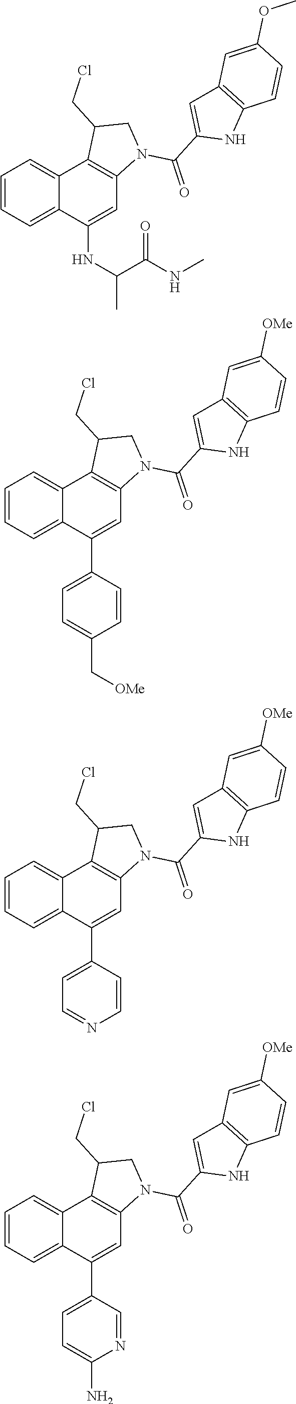 [SMILES: CNC(=O)C(C)Nc1cc2c(c3ccccc13)C(CCl)CN2C(=O)c1cc2cc(OC)ccc2[nH]1 ; COCc1ccc(-c2cc3c(c4ccccc24)C(CCl)CN3C(=O)c2cc3cc(OC)ccc3[nH]2)cc1 ; COc1ccc2[nH]c(C(=O)N3CC(CCl)c4c3cc(-c3ccncc3)c3ccccc43)cc2c1 ; COc1ccc2[nH]c(C(=O)N3CC(CCl)c4c3cc(-c3ccc(N)nc3)c3ccccc43)cc2c1]

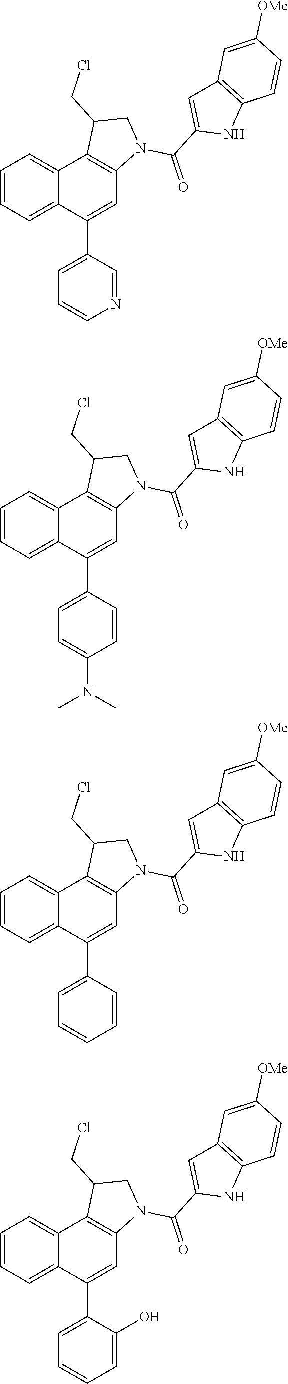 [SMILES: COc1ccc2[nH]c(C(=O)N3CC(CCl)c4c3cc(-c3cccnc3)c3ccccc43)cc2c1 ; COc1ccc2[nH]c(C(=O)N3CC(CCl)c4c3cc(-c3ccc(N(C)C)cc3)c3ccccc43)cc2c1 ; COc1ccc2[nH]c(C(=O)N3CC(CCl)c4c3cc(-c3ccccc3)c3ccccc43)cc2c1 ; COc1ccc2[nH]c(C(=O)N3CC(CCl)c4c3cc(-c3ccccc3O)c3ccccc43)cc2c1]

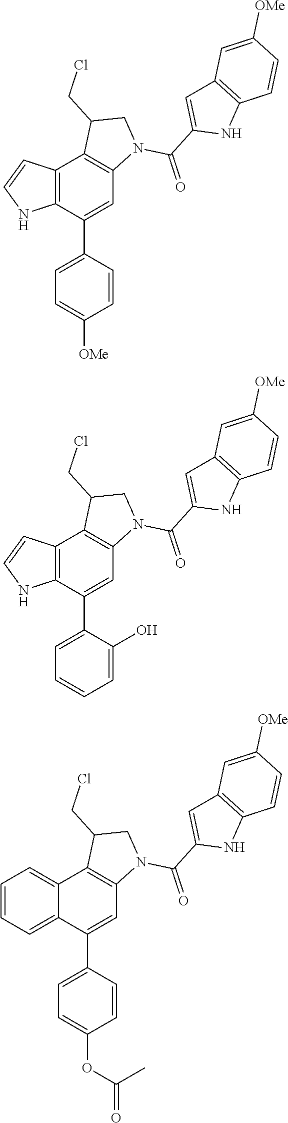 [SMILES: COc1ccc(-c2cc3c(c4cc[nH]c24)C(CCl)CN3C(=O)c2cc3cc(OC)ccc3[nH]2)cc1 ; COc1ccc2[nH]c(C(=O)N3CC(CCl)c4c3cc(-c3ccccc3O)c3[nH]ccc43)cc2c1 ; COc1ccc2[nH]c(C(=O)N3CC(CCl)c4c3cc(-c3ccc(OC(C)=O)cc3)c3ccccc43)cc2c1]

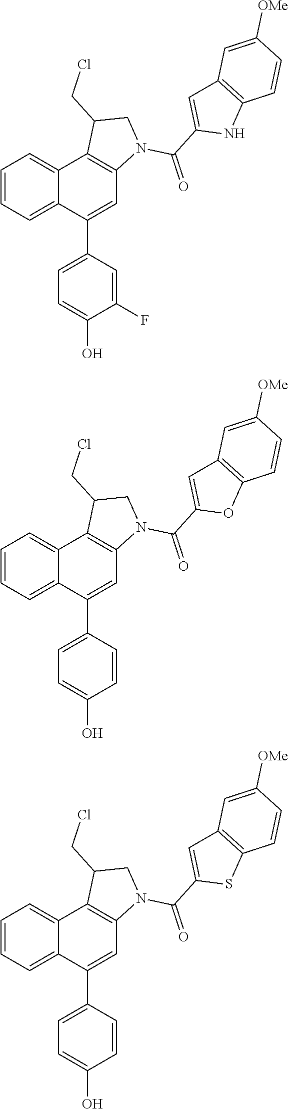 [SMILES: COc1ccc2[nH]c(C(=O)N3CC(CCl)c4c3cc(-c3ccc(O)c(F)c3)c3ccccc43)cc2c1 ; COc1ccc2oc(C(=O)N3CC(CCl)c4c3cc(-c3ccc(O)cc3)c3ccccc43)cc2c1 ; COc1ccc2sc(C(=O)N3CC(CCl)c4c3cc(-c3ccc(O)cc3)c3ccccc43)cc2c1]

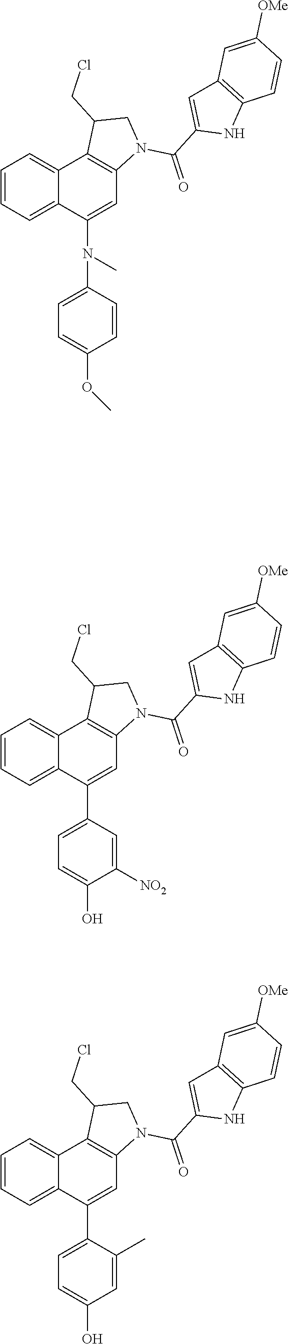 [SMILES: COc1ccc(N(C)c2cc3c(c4ccccc24)C(CCl)CN3C(=O)c2cc3cc(OC)ccc3[nH]2)cc1 ; COc1ccc2[nH]c(C(=O)N3CC(CCl)c4c3cc(-c3ccc(O)c([N+](=O)[O-])c3)c3ccccc43)cc2c1 ; COc1ccc2[nH]c(C(=O)N3CC(CCl)c4c3cc(-c3ccc(O)cc3C)c3ccccc43)cc2c1]

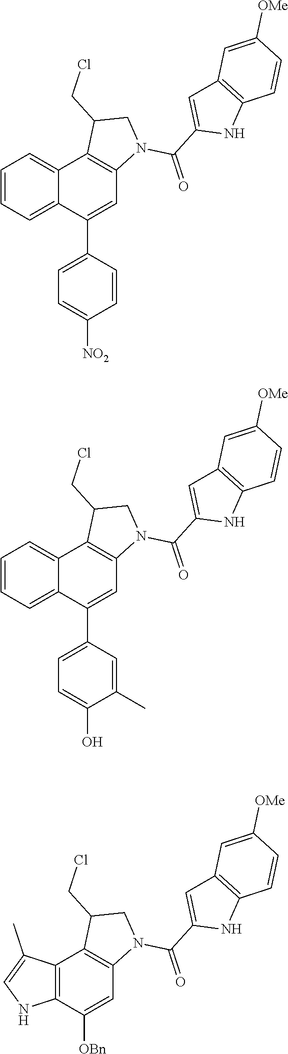 [SMILES: COc1ccc2[nH]c(C(=O)N3CC(CCl)c4c3cc(-c3ccc([N+](=O)[O-])cc3)c3ccccc43)cc2c1 ; COc1ccc2[nH]c(C(=O)N3CC(CCl)c4c3cc(-c3ccc(O)c(C)c3)c3ccccc43)cc2c1 ; COc1ccc2[nH]c(C(=O)N3CC(CCl)c4c3cc(OCc3ccccc3)c3[nH]cc(C)c43)cc2c1]

In certain embodiments the compound of Formula I is selected from:

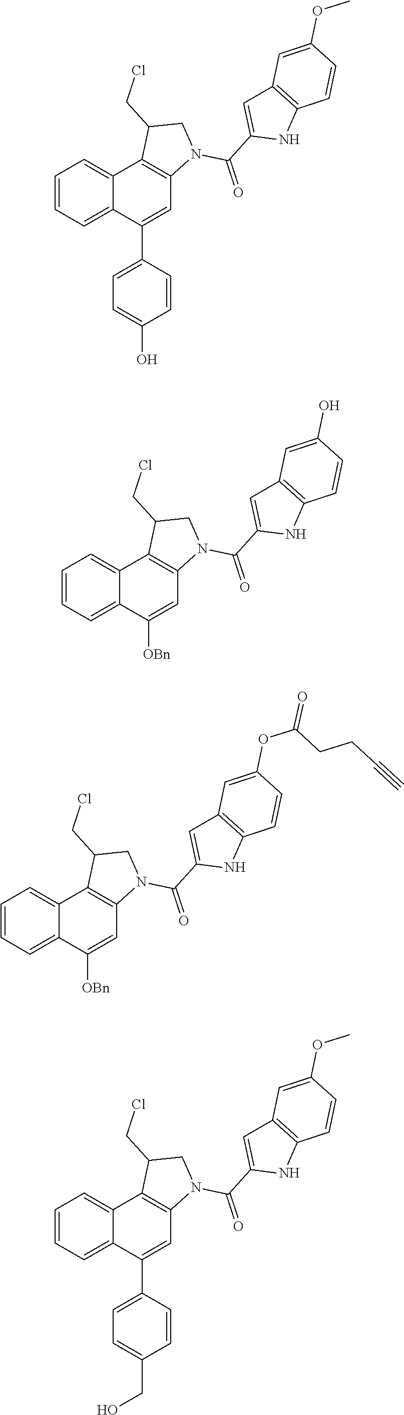 [SMILES: COc1ccc2[nH]c(C(=O)N3CC(CCl)c4c3cc(-c3ccc(O)cc3)c3ccccc43)cc2c1 ; O=C(c1cc2cc(O)ccc2[nH]1)N1CC(CCl)c2c1cc(OCc1ccccc1)c1ccccc21 ; C#CCCC(=O)Oc1ccc2[nH]c(C(=O)N3CC(CCl)c4c3cc(OCc3ccccc3)c3ccccc43)cc2c1 ; COc1ccc2[nH]c(C(=O)N3CC(CCl)c4c3cc(-c3ccc(CO)cc3)c3ccccc43)cc2c1]

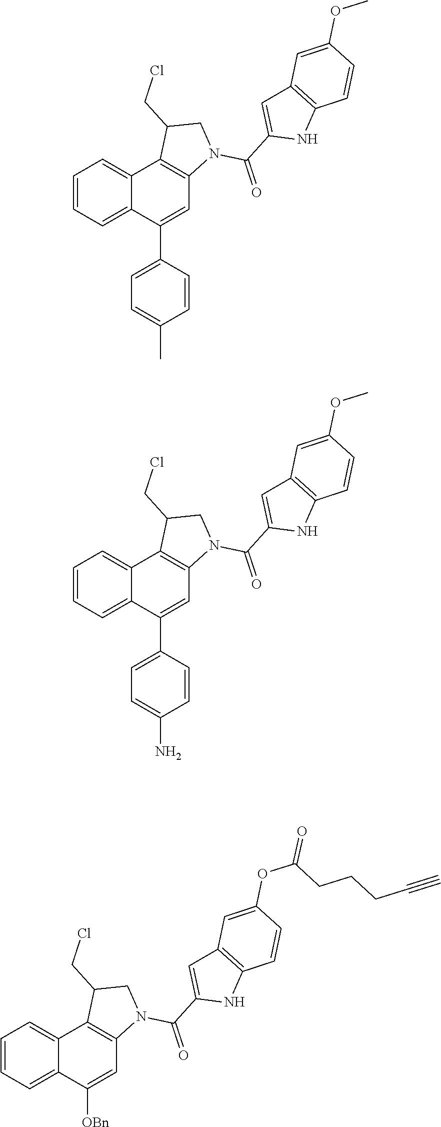 [SMILES: COc1ccc2[nH]c(C(=O)N3CC(CCl)c4c3cc(-c3ccc(C)cc3)c3ccccc43)cc2c1 ; COc1ccc2[nH]c(C(=O)N3CC(CCl)c4c3cc(-c3ccc(N)cc3)c3ccccc43)cc2c1 ; C#CCCCC(=O)Oc1ccc2[nH]c(C(=O)N3CC(CCl)c4c3cc(OCc3ccccc3)c3ccccc43)cc2c1]

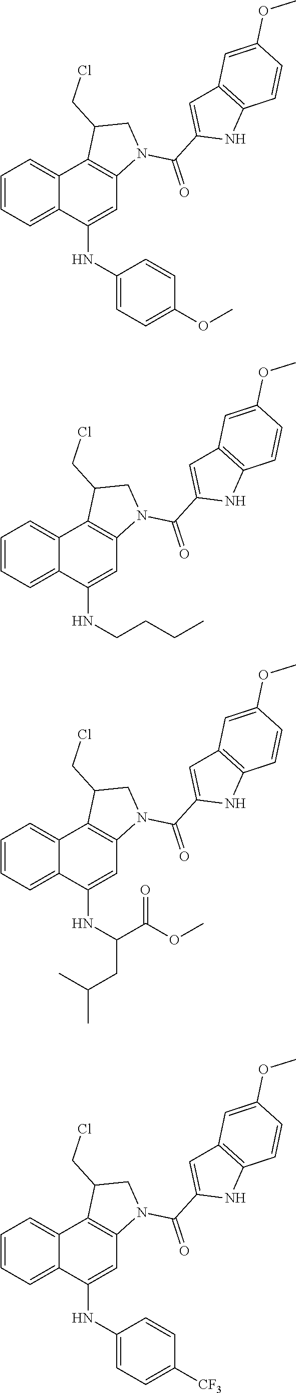 [SMILES: COc1ccc(Nc2cc3c(c4ccccc24)C(CCl)CN3C(=O)c2cc3cc(OC)ccc3[nH]2)cc1 ; CCCCNc1cc2c(c3ccccc13)C(CCl)CN2C(=O)c1cc2cc(OC)ccc2[nH]1 ; COC(=O)C(CC(C)C)Nc1cc2c(c3ccccc13)C(CCl)CN2C(=O)c1cc2cc(OC)ccc2[nH]1 ; COc1ccc2[nH]c(C(=O)N3CC(CCl)c4c3cc(Nc3ccc(C(F)(F)F)cc3)c3ccccc43)cc2c1]

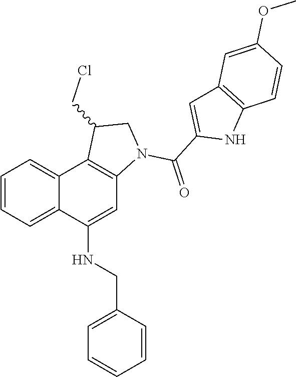 [SMILES: COc1ccc2[nH]c(C(=O)N3CC(CCl)c4c3cc(NCc3ccccc3)c3ccccc43)cc2c1]

In another aspect of the invention there is provided a compound of formula I for use as a medicament.

In another aspect a compound of formula I is for use in the treatment of a condition treatable by the administration of a cytotoxic agent.

In another aspect a compound of formula I is for use in the treatment cancer.

In another aspect there is provided a method for the treatment of a condition treatable by the administration of a cytotoxic agent, wherein the method comprises administering a pharmaceutically effective amount of a compound of formula I.

In another aspect there is provided a method for the treatment of cancer, wherein the method comprises administering a pharmaceutically effective amount of a compound of formula I.

In certain embodiments the condition treatable by the administration of a cytotoxic agent is cancer. The cancer treatable in any aspect or embodiment of the present invention may be solid tumors, lymphoma or leukemia. Solid tumours, may be selected from, but not limited to cancers of the breast, lung, prostate, colon, bladder, brain, pancreas, head & neck and neuroblastoma. In certain embodiments the cancer is breast cancer.

In another aspect of the present invention there is provided a compound of the present invention which is suitable for conjugation with an antibody, wherein, for the sake of future brevity when discussing drug conjugates, “antibody” includes the normal meaning of antibody in addition to biosimilar, affimer or other non-antibody binding proteins. As such, the present invention provides the use of the compound of the present invention in the manufacture of an antibody-drug conjugate (meaning an antibody, biosimilar, affimer or other non-antibody binding proteins drug conjugate). The antibody-drug conjugate can be prepared by substituting a compound of the present invention with a linker group and attaching an antibody to the linker group or substituted the compound of the invention with a linker-antibody conjugate.

In an aspect of the present invention there is provide an antibody-drug conjugate, wherein the drug is a compound of the present invention. In another aspect of the present invention there is provided an antibody-drug conjugate comprising a compound of Formula I, an antibody, biosimilar, affimer or other non-antibody binding proteins, and a linker connecting the compound of Formula I to the antibody.

In certain embodiments the linker is attached to the compound of Formula I at positions 4, 5, 6, or 7 shown in the structure below, or on any chemically possible position on the R¹ group:

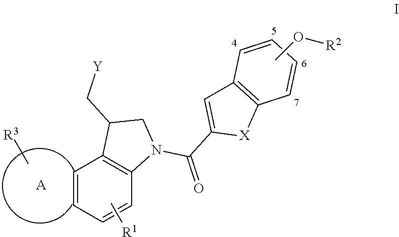

Any linker known in the art will be suitable for use in the present invention. The linker must be suitable for connection to the compound of Formula I and suitable for connection to the antibody. As such, the linker will generally be bi-functional with a first functional group at a first end of a molecule and a second functional group at a second end of the molecule. The linker first and/or second functional group may be an ether, an amine, an ester, a carbonate, a carbamate, a thioether or other sulphur bridging unit, or a triazole formed from click chemistry. The molecule of the linker may comprise an alkyl chain, alkenyl chain or alkynyl chain, polyethylene glycol (or other similar moiety for the purpose of enhancing aqueous solubility), or a peptide. The alkyl chain, alkenyl chain or alkynyl chain may be any length, optionally from 3 to 10 atoms in length. The polyethylene glycol may consist of 2 to 10 repeating units. The peptide may consist of 1 to 6 amino acid residues.

The linker may comprise a self-elimination module, a cleavable peptide, and an antibody bonding unit. These features of a linker within the context of an antibody-drug conjugate are well established and known to the person skilled in the art.

The linker may be cleavable (for example by enzymatic or chemical means) or non-cleavable.

For example, the use of antibody-drug conjugates with linkers that would be contemplated by the present invention are discussed in: Yao et al, Drug Discovery Today, 2021, 26(8), 1857-1874; Jukes et al, Drug Discovery Today, 2021, 26(2), 577-584; Khongorzul et al., 2020, 18(1), 3-19; Dumontet, Nat Revs Drug Discov, 2023, 22, 641-661; Dokter et al, Mol. Cancer Ther. 2014, 13(11), 2618-2629; and van der Lee, Mol Cancer Ther, 2015, 14(3), 692-703, all of which are incorporated herein by reference.

Linkers that may be suitable for the present application are also discussed in U.S. Pat. No. 7,964,566 and US 2017/0232108, both of which are incorporated by reference.

### BRIEF DESCRIPTION OF THE DRAWINGS

Embodiments of the invention are further described hereinafter with reference to the accompanying drawings, in which:

FIG. 1 is a Western Blot showing H2AX phosphorylation as a marker of double-stranded DNA damage.

FIG. 2 is a Western Blot showing expression of γ-H2AX phosphorylations as a marker of DNA damage in MCF-7 cells following treatment with duocarmycin compounds

### DETAILED DESCRIPTION

Given below are definitions of terms used in this application. Any term not defined herein takes the normal meaning as the skilled person would understand the term.

The term “halo” refers to one of the halogens, group 17 of the periodic table. In particular the term refers to fluorine, chlorine, bromine and iodine. Preferably, the term refers to fluorine or chlorine.

The term “C₁₋₆ alkyl” refers to a linear or branched hydrocarbon chain containing 1, 2, 3, 4, 5 or 6 carbon atoms, for example methyl, ethyl, n-propyl, iso-propyl, n-butyl, sec-butyl, tert-butyl, n-pentyl and n-hexyl. The alkyl group may be unsubstituted or substituted by one or more substituents. Possible substituents are described below. Substituents for the alkyl group may be halogen, e.g. fluorine, chlorine, bromine and iodine, OH, C₁₋₆ alkoxy.

The term “C₁₋₆ alkoxy” refers to an alkyl group which is attached to a molecule via oxygen. This includes moieties where the alkyl part may be linear or branched and may contain 1, 2, 3, 4, 5 or 6 carbon atoms, for example methyl, ethyl, n-propyl, iso-propyl, n-butyl, sec-butyl, tert-butyl, n-pentyl and n-hexyl. Therefore, the alkoxy group may be methoxy, ethoxy, n-propoxy, iso-propoxy, n-butoxy, sec-butoxy, tert-butoxy, n-pentoxy and n-hexoxy. The alkyl part of the alkoxy group may be unsubstituted or substituted by one or more substituents. Possible substituents are described below. Substituents for the alkyl group may be halogen, e.g. fluorine, chlorine, bromine and iodine, OH, C₁₋₆ alkoxy.

The term “C₁₋₆ haloalkyl” refers to a hydrocarbon chain substituted with at least one halogen atom independently chosen at each occurrence, for example fluorine, chlorine, bromine and iodine. The halogen atom may be present at any position on the hydrocarbon chain. For example, C₁₋₆ haloalkyl may refer to chloromethyl, flouromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, or trifluoropropyl.

The term “C₂₋₆ alkenyl” refers to a branched or linear hydrocarbon chain containing at least one double bond and having 2, 3, 4, 5 or 6 carbon atoms. The double bond(s) may be present as the E or Z isomer. The double bond may be at any possible position of the hydrocarbon chain. For example, the “C₂₋₆ alkenyl” may be ethenyl, propenyl, butenyl, butadienyl, pentenyl, pentadienyl, hexenyl and hexadienyl.

The term “C₂₋₆ alkynyl” refers to a branded or linear hydrocarbon chain containing at least one triple bond and having 2, 3, 4, 5 or 6 carbon atoms. The triple bond may be at any possible position of the hydrocarbon chain. For example, the “C₂₋₆ alkynyl” may be ethynyl, propynyl, butynyl, pentynyl and hexynyl.

The term “carbocyclic” refers to a saturated or unsaturated carbon containing ring system. A “carbocyclic” system may be monocyclic or a fused polycyclic ring system, for example, bicyclic or tricyclic. A “carbocyclic” moiety may contain from 3 to 14 carbon atoms, for example, 3 to 8 carbon atoms in a monocyclic system and 7 to 14 carbon atoms in a polycyclic system. “Carbocyclic” encompasses cycloalkyl moieties, cycloalkenyl moieties, aryl ring systems and fused ring systems including an aromatic portion.

The term “heterocyclic” refers to a saturated or unsaturated ring system containing at least one heteroatom selected from N, O or S. A “heterocyclic” system may contain 1, 2, 3 or 4 heteroatoms, for example 1 or 2. A “heterocyclic” system may be monocyclic or a fused polycyclic ring system, for example, bicyclic or tricyclic. A “heterocyclic” moiety may contain from 3 to 14 carbon atoms, for example, 3 to 8 carbon atoms in a monocyclic system and 7 to 14 carbon atoms in a polycyclic system. “Heterocyclic” encompasses heterocycloalkyl moieties, heterocycloalkenyl moieties and heteroaromatic moieties. For example, the heterocyclic group may be: oxirane, aziridine, azetidine, oxetane, tetrahydrofuran, pyrrolidine, imidazolidine, succinimide, pyrazolidine, oxazolidine, isoxazolidine, thiazolidine, isothiazolidine, piperidine, morpholine, thiomorpholine, piperazine, and tetrahydropyran.

The term “cycloalkyl” refers to a saturated hydrocarbon ring system. The “cycloalkyl” group may be denoted as a “C₃₋₁₀ cycloalkyl” containing 3, 4, 5, 6, 7, 8, 9 or 10 carbon atoms. The ring system may be a single ring or a bi-cyclic or tri-cyclic ring system. For example, the “cycloalkyl” may be cyclopropyl, cyclobutyl, cyclopentyl, cyclohexyl, bicyclohexyl, cycloheptyl and cyclooctyl.

The term “cycloalkenyl” refers to an unsaturated hydrocarbon ring system that is not aromatic. The “cycloalkenyl” group may be denoted as a “C₃₋₁₀ cycloalkenyl”. A “C₃₋₁₀ cycloalkenyl” is a ring system containing 3, 4, 5, 6, 7, 8, 9 or 10 carbon atoms. The ring may contain more than one double bond provided that the ring system is not aromatic. The ring system may be a single ring or a bi-cyclic or tri-cyclic ring system. For example, the “cycloalkenyl” may be cyclopropenyl, cyclobutenyl, cyclopentenyl, cyclopentadienyl, cyclohexenyl, cyclohexadienly, cycloheptenyl, cycloheptadiene, cyclooctenyl and cycloatadienyl.

The term “heterocycloalkyl” refers to a saturated hydrocarbon ring system with at least one heteroatom within the ring selected from N, O and S. The “heterocycloalkyl” group may be denoted as a “C₃₋₁₀ heterocycloalkyl”. A “C₃₋₁₀ heterocycloalkyl” is a ring system containing 3, 4, 5, 6, 7, 8, 9 or 10 atoms at least one being a heteroatom. For example there may be 1, 2 or 3 heteroatoms, optionally 1 or 2. The “heterocycloalkyl” group may also be denoted as a “3 to 10 membered heterocycloalkyl” which is also a ring system containing 3, 4, 5, 6, 7, 8, 9 or 10 atoms at least one being a heteroatom. The ring system may be a single ring or a bi-cyclic or tri-cyclic ring system. Where the ring system is bicyclic one of the rings may be an aromatic ring, for example as in indane. The “heterocycloalkyl” may be bonded to the rest of the molecule through any carbon atom or heteroatom. The “heterocycloalkyl” may have one or more, e.g. one or two, bonds to the rest of the molecule: these bonds may be through any of the atoms in the ring. For example, the “heterocycloalkyl” may be oxirane, aziridine, azetidine, oxetane, tetrahydrofuran, pyrrolidine, imidazolidine, succinimide, pyrazolidine, oxazolidine, isoxazolidine, thiazolidine, isothiazolidine, piperidine, morpholine, thiomorpholine, piperazine, tetrahydropyran, and indane.

The term “heterocycloalkenyl” refers to an unsaturated hydrocarbon ring system, that is not aromatic, having at least one heteroatom within the ring selected from N, O and S.

The “heterocycloalkenyl” group may be denoted as a “C₃₋₁₀ heterocycloalkenyl”. A “C₃₋₁₀ heterocycloalkenyl” is a ring system containing 3, 4, 5, 6, 7, 8, 9 or 10 atoms at least one being a heteroatom. For example there may be 1, 2 or 3 heteroatoms, optionally 1 or 2. The “heterocycloalkenyl” group may also be denoted as a “3 to 10 membered heterocycloalkenyl” which is also a ring system containing 3, 4, 5, 6, 7, 8, 9 or 10 atoms at least one being a heteroatom. The ring system may be a single ring or a bi-cyclic or tri-cyclic ring system.

Where the ring system is bicyclic one of the rings may be an aromatic ring, for example as in indoline and dihydrobenzofuran. The “heterocycloalkenyl” may be bonded to the rest of the molecule through any carbon atom or heteroatom. The “heterocycloalkenyl” may have one or more, e.g. one or two, bonds to the rest of the molecule: these bonds may be through any of the atoms in the ring. For example, the “C₃₋₈ heterocycloalkenyl” may be tetrahydropyridine, dihydropyran, dihydrofuran, pyrroline, dihydrobenzofuran, dihydrobenzothiophene and indoline.

The term “aromatic” when applied to a substituent as a whole means a single ring or polycyclic ring system with 4n+2 electrons in a conjugated u system within the ring or ring system where all atoms contributing to the conjugated u system are in the same plane.

The term “aryl” refers to an aromatic hydrocarbon ring system. The ring system has 4n+2 electrons in a conjugated u system within a ring where all atoms contributing to the conjugated u system are in the same plane. The ring system may be a single ring or a bi-cyclic or tri-cyclic ring system. For example, the “aryl” may be phenyl and naphthyl. The aryl system itself may be substituted with other groups.

The term “heteroaryl” refers to an aromatic hydrocarbon ring system with at least one heteroatom within a single ring or within a fused ring system, selected from O, N and S.

The ring or ring system has 4n+2 electrons in a conjugated u system where all atoms contributing to the conjugated u system are in the same plane. The ring system may be a single ring or a bi-cyclic or tri-cyclic ring system. For example, the “heteroaryl” may be imidazole, thiene, furane, thianthrene, pyrrol, benzimidazole, pyrazole, pyrazine, pyridine, pyrimidine and indole.

The term “alkaryl” refers to an aryl group, as defined above, bonded to a C₁₋₄ alkyl, where the C₁₋₄ alkyl group provides attachment to the remainder of the molecule.

The term “alkheteroaryl” refers to a heteroaryl group, as defined above, bonded to a C1-4 alkyl, where the alkyl group provides attachment to the remainder of the molecule.

The term “halogen” herein includes reference to F, Cl, Br and I. Halogen may be Cl. Halogen may be F.

A bond terminating inside a cyclic structure and not terminating at an atom of the ring structure represents that the bond may be connected to any of the atoms in the ring structure where allowed by valency.

Where a moiety is substituted, it may be substituted at any point on the moiety where chemically possible and consistent with atomic valency requirements. The moiety may be substituted as defined elsewhere herein or may be substituted by one or more substituents, e.g. 1, 2, 3 or 4 substituents; optionally there are 1 or 2 substituents on a group.

Where there are two or more substituents, the substituents may be the same or different. The substituent(s) may be selected from: —OH, —NH₂, amidino, guanidino, hydroxyguanidino, formamidino, isothioureido, ureido, mercapto, C(O)H, acyl, acyloxy, carboxy, sulfo, sulfamoyl, carbamoyl, cyano, azo, nitro, halo, C₁₋₃ alkyl, C₁₋₃ alkoxy, C₁₋₃ haloalkyl, C₃₋₈ cycloalkyl, C₂₋₆ alkenyl, C₂₋₆ alkynyl, aryl, heteroaryl or alkaryl. Where the group to be substituted is an alkyl group the substituent may be ═O.

If chemically possible to do so, a cyclic substituent may be substituted on a group so as to form a spiro-cycle.

Substituents are only present at positions where they are chemically possible, the person skilled in the art being able to decide (either experimentally or theoretically) without inappropriate effort which substitutions are chemically possible and which are not.

In embodiments where there is a single enantiomer of the compounds of the invention, the compounds of the invention may have an enantiomeric purity of at least about 90% enantiomeric excess (ee), at least about 95% enantiomeric excess (ee), at least about 98% enantiomeric excess (ee), at least about 99% enantiomeric excess (ee), or 100% enantiomeric excess (ee). In embodiments where there is a mixture of enantiomers of the compounds of the invention, the compounds of the invention may be a racemic mixture or any other mixture of enantiomers, for example the compounds of the invention may have an enantiomeric purity of at least about 50% enantiomeric excess (ee), at least about 60% enantiomeric excess (ee), at least about 70% enantiomeric excess (ee), at least about 80% enantiomeric excess (ee), at least about 90% enantiomeric excess (ee), or at least about 95% enantiomeric excess (ee).

Throughout the description the disclosure of a compound also encompasses pharmaceutically acceptable salts, solvates and stereoisomers thereof. Where a compound has a stereocentre, both (R) and (S) stereoisomers are contemplated by the invention, equally mixtures of stereoisomers or a racemic mixture are contemplated by the present application. Where a compound of the invention has two or more stereocentres any combination of (R) and (S) stereoisomers is contemplated. The combination of (R) and (S) stereoisomers may result in a diastereomeric mixture or a single diastereoisomer. The compounds of the invention may be present as a single stereoisomer or may be mixtures of stereoisomers, for example racemic mixtures and other enantiomeric mixtures, and diasteroemeric mixtures. Where the mixture is a mixture of enantiomers the enantiomeric excess may be any of those disclosed above. Where the compound is a single stereoisomer the compounds may still contain other diasteroisomers or enantiomers as impurities. Hence a single stereoisomer does not necessarily have an enantiomeric excess (e.e.) or diastereomeric excess (d.e.) of 100% but could have an e.e. or d.e. of about at least 85%

The invention contemplates pharmaceutically acceptable salts of the compounds of formula I. These may include the acid addition or base salts of the compounds. These may be acid addition and base salts of the compounds. In addition the invention contemplates solvates of the compounds. These may be hydrates or other solvated forms of the compound.

Suitable acid addition salts are formed from acids which form non-toxic salts. Examples include the acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 1,5-naphthalenedisulfonate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts. For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of formula (I) may be prepared by one or more of three methods:
(i) by reacting the compound of formula (I) with the desired acid or base;
(ii) by removing an acid- or base-labile protecting group from a suitable precursor of the compound of formula (I) or by ring-opening a suitable cyclic precursor, for example, a lactone or lactam, using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Hereinafter all references to compounds of any formula include references to salts, solvates and complexes thereof and to solvates and complexes of salts thereof.

The compounds of the invention include compounds of a number of formula as herein defined, including all polymorphs and crystal habits thereof, prodrugs and isomers thereof (including optical, geometric and tautomeric isomers) as hereinafter defined and isotopically-labeled compounds of the invention.

Before purification, the compounds of the present invention may exist as a mixture of enantiomers depending on the synthetic procedure used. The enantiomers can be separated by conventional techniques known in the art. Thus the invention covers individual enantiomers as well as mixtures thereof.

For some of the steps of the process of preparation of the compounds of formula (I), it may be necessary to protect potential reactive functions that are not wished to react, and to cleave said protecting groups in consequence. In such a case, any compatible protecting radical can be used. In particular methods of protection and deprotection such as those described by T. W. GREENE (Protective Groups in Organic Synthesis, A. Wiley-Interscience Publication, 1981) or by P. J. Kocienski (Protecting groups, Georg Thieme Verlag, 1994), can be used. All of the above reactions and the preparations of novel starting materials used in the preceding methods are conventional and appropriate reagents and reaction conditions for their performance or preparation as well as procedures for isolating the desired products will be well-known to those skilled in the art with reference to literature precedents and the examples and preparations hereto.

Also, the compounds of the present invention as well as intermediates for the preparation thereof can be purified according to various well-known methods, such as for example crystallization or chromatography. [00178] The method of treatment or the compound for use in the treatment of cancer, lymphoma, leukemia or immunological diseases as defined hereinbefore may be applied as a sole therapy or be a combination therapy with an additional active agent.

The method of treatment or the compound for use in the treatment of cancer, lymphoma or leukemia may involve, in addition to the compound of the invention, conventional surgery or radiotherapy or chemotherapy. Such chemotherapy may include one or more of the following categories of anti-tumor agents:
(i) antiproliferative/antineoplastic drugs and combinations thereof, such as alkylating agents (for example cis-platin, oxaliplatin, carboplatin, cyclophosphamide, nitrogen mustard, bendamustin, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, pemetrexed, cytosine arabinoside, and hydroxyurea); antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like paclitaxel and docetaxel and polokinase inhibitors); proteasome inhibitors, for example carfilzomib and bortezomib; interferon therapy; and topoisomerase inhibitors (for example epipodophyllotoxins like etoposide and teniposide, amsacrine, topotecan, mitoxantrone and camptothecin);
(ii) cytostatic agents such as antiestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5-alpha-reductase such as finasteride;
(iii) anti-invasion agents, for example dasatinib and bosutinib (SKI-606), and metalloproteinase inhibitors, inhibitors of urokinase plasminogen activator receptor function or antibodies to heparanase;
(iv) inhibitors of growth factor function: for example such inhibitors include growth factor antibodies and growth factor receptor antibodies, for example the anti-erbB2 antibody trastuzumab [Herceptin™], the anti-EGFR antibody panitumumab, the anti-erbB1 antibody cetuximab, tyrosine kinase inhibitors, for example inhibitors of the epidermal growth factor family (for example EGFR family tyrosine kinase inhibitors such as gefitinib, erlotinib and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; modulators of protein regulators of cell apoptosis (for example Bcl-2 inhibitors); inhibitors of the platelet-derived growth factor family such as imatinib and/or nilotinib (AMN107); inhibitors of serine/threonine kinases (for example Ras/Raf signalling inhibitors such as famesyl transferase inhibitors, for example sorafenib, tipifamib and lonafamib), inhibitors of cell signalling through MEK and/or AKT kinases, c-kit inhibitors, abl kinase inhibitors, PI3 kinase inhibitors, Plt3 kinase inhibitors, CSF-1 R kinase inhibitors, IGF receptor, kinase inhibitors; aurora kinase inhibitors and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastinm); thalidomide; lenalidomide; and for example, a VEGF receptor tyrosine kinase inhibitor such as vandetanib, vatalanib, sunitinib, axitinib and pazopanib; (vi) gene therapy approaches, including for example approaches to replace aberrant genes such as aberrant p53 or aberrant BRCA1 or BRCA2;
(vii) immunotherapy approaches, including for example antibody therapy such as alemtuzumab, rituximab, ibritumomab tiuxetan (Zevalin®) and ofatumumab; interferons such as interferon a; interleukins such as IL-2 (aldesleukin); interleukin inhibitors for example IRAK4 inhibitors; cancer vaccines including prophylactic and treatment vaccines such as HPV vaccines, for example Gardasil, Cervarix, Oncophage and Sipuleucel-T (Provenge); and toll-like receptor modulators for example TLR-7 or TLR-9 agonists; and
(viii) cytotoxic agents for example fludaribine (fludara), cladribine, pentostatin (Nipent™);
(ix) steroids such as corticosteroids, including glucocorticoids and mineralocorticoids, for example aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, methylprednisolone, mometasone paramethasone, mometasone furoate monohydrate, prednicarbate, prednisolone, prednisone, tixocortol, tixocortol pivalate, triamcinolone, triamcinolone acetonide, triamcinolone alcohol and their respective pharmaceutically acceptable derivatives. A combination of steroids may be used, for example a combination of two or more steroids mentioned in this paragraph;
(x) targeted therapies, for example PI3Kd inhibitors, for example idelalisib and perifosine.

Such combination treatment may be achieved byway of the simultaneous, sequential or separate dosing of the individual components of the treatment. Such combination products employ the compounds of this invention within a therapeutically effective dosage range described hereinbefore and the other pharmaceutically-active agent within its approved dosage range.

According to a further aspect of the invention there is provided a pharmaceutical product comprising a compound of formula (I), or a pharmaceutically acceptable salt thereof as defined hereinbefore and an additional active agent. The additional active agent may be an anti-tumour agent as defined hereinbefore for the combination treatment of cancer.

According to a further aspect of the invention there is provided a method of treating a condition treatable by a cytotoxic agent comprising administering a therapeutically effective amount of a compound of formula (I), or a pharmaceutically acceptable salt thereof simultaneously, sequentially or separately with an additional anti-tumor agent, as defined hereinbefore, to a patient in need thereof.

According to a further aspect of the invention there is provided a compound of formula (I), or a pharmaceutically acceptable salt thereof for use simultaneously, sequentially or separately with an additional anti-tumour agent as defined hereinbefore, in the treatment of a condition treatable by a cytotoxic agent.

According to another aspect of the invention there is provided a use of the compound of formula (I) in combination with an anti-tumor agent as hereinbefore described.

The compound of formula (I) may be used simultaneously, sequentially or separately with the additional anti-tumor agent The use may be in a single combination product comprising the compound of formula (I) and the anti-tumor agent.

According to a further aspect there is provided a method of providing a combination product, wherein the method comprises providing a compound of formula (I) simultaneously, sequentially or separately with an anti-tumor agent, as defined hereinbefore. The method may comprise combining the compound of formula (I) and the anti-tumor agent in a single dosage form. Alternatively the method may comprise providing the anti-tumor agent as separate dosage forms.

According to a further aspect there is provided a method of providing a combination product, wherein the method comprises providing a compound of formula (I) simultaneously, sequentially or separately with an anti-tumor agent, as defined hereinbefore. The method may comprise combining the compound of formula (I) and the anti-tumor agent in a single dosage form. Alternatively the method may comprise providing the anti-tumor agent as separate dosage forms.

### EXAMPLES

Compounds of the present invention were produced by the following methods.

### General Method A:

### General Method for Synthesis of 5-alkyl/arylamino-(1-(chloromethyl)-1,2-dihydro-3H-benzo[e]indol-3-yl)(5-methoxy-1H-indol-2-yl)methanone

To a mixture of 5-triflyloxy-(1-(chloromethyl)-1,2-dihydro-3H-benzo[e]indol-3-yl)(5-methoxy-1H-indol-2-yl)methanone (27 mg, 0.05 mmol), potassium phosphate (12 mg, 0.1 mmol), Xantphos (12 mg, 0.02 mmol), and the appropriate alkyl or arylamino derivative (0.15 mmol) in dry toluene (1.5 mL) was added Pd(OAc)₂ (2.2 mg, 0.01 mmol) in an oven-dried pressure tube under nitrogen. The reaction mixture was stirred and refluxed at 110-125° C. for 1-4 h in oil bath. The crude reaction mixture was applied directly to a silica gel column to afford the purified product.

### Example 1—DP58

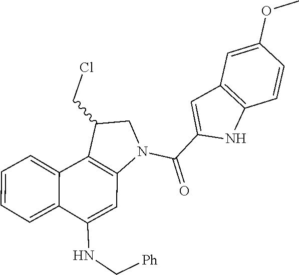 [SMILES: COc1ccc2[nH]c(C(=O)N3CC(CCl)c4c3cc(NCc3ccccc3)c3ccccc43)cc2c1]

HPLC-MS: m/z (%)=496 [M+H], 498 [M+2+H]

LC/MS analysis was performed using a Waters analytical system, comprising the following modules: Waters Alliance 2695 Separations Module, Waters 996 PDA Detector, and Waters Micromass ZQ Mass Detector.

Column: Hichrom RPB-150 AM, 2.1×150 mm, 3.5 μm; mobile phase A: 90% water, 10% MeOH+0.1% formic acid; mobile phase B: 90% MeOH, 10% water+0.1% Formic acid; flow rate 0.25 ml/min.

Solvent gradient:

| Time | Mobile Phase A | Mobile Phase B |
|---|---|---|
| (min) | (%) | (%) |
| 0 | 95 | 5 |
| 5 | 50 | 50 |
| 28 | 0 | 100 |
| 30 | 0 | 100 |
| 35 | 95 | 5 |

1H NMR (CDCl₃) δ: 9.47 (s, 1H), 7.88 (s, 1H), 7.81 (d, J=8 Hz, 1H), 7.72 (dd, J=8/0.8 Hz 1H), 7.52 (m, 1H), 7.45 (m, 2H), 7.36 (m, 5H), 7.13 (d, J=2.4 Hz, 1H), 7.04 (dd, J=2.8/0.8 Hz 1H), 7.00 (dd, J=6.4/2.4 Hz 1H), 4.79 (m, 2H), 4.64 (m, 1H), 4.52 (m, 2H), 4.09 (m, 1H), 3.96 (m, 1H), 3.87 (s, 3H, OMe), 3.44 (t, J=10.8 Hz 1H).

### Example 2—DP-59

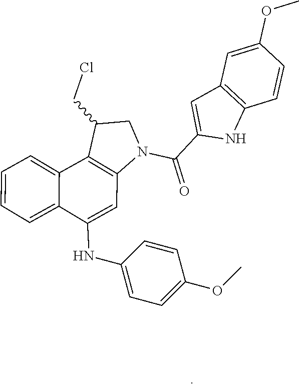 [SMILES: COc1ccc(Nc2cc3c(c4ccccc24)C(CCl)CN3C(=O)c2cc3cc(OC)ccc3[nH]2)cc1]

HPLC-MS: m/z (%)=512 [M+H], 514 [M+2+H]

### Example 3—DP-63

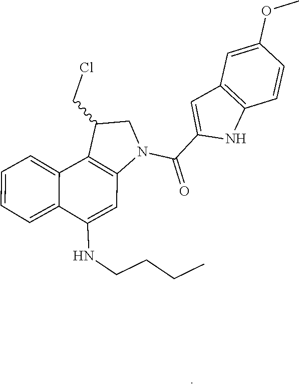 [SMILES: CCCCNc1cc2c(c3ccccc13)C(CCl)CN2C(=O)c1cc2cc(OC)ccc2[nH]1]

HPLC-MS: m/z (%)=462 [M+H], 464 [M+2+H]

### Example 4—DP-65

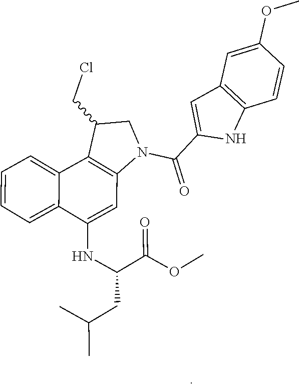 [SMILES: COC(=O)[C@H](CC(C)C)Nc1cc2c(c3ccccc13)C(CCl)CN2C(=O)c1cc2cc(OC)ccc2[nH]1]

HPLC-MS: m/z (%)=534 [M+H], 536 [M+2+H]

### Example 5—DP-70

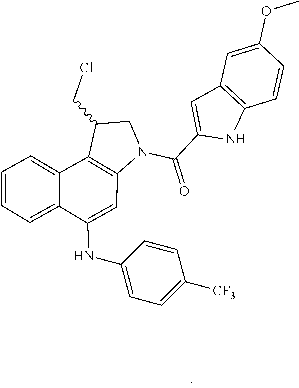 [SMILES: COc1ccc2[nH]c(C(=O)N3CC(CCl)c4c3cc(Nc3ccc(C(F)(F)F)cc3)c3ccccc43)cc2c1]

HPLC-MS: m/z (%)=550 [M+H], 552 [M+2+H]

### General Method B:

### General Method for Synthesis of 5-aryl-(1-(chloromethyl)-1,2-dihydro-3H-benzo[e]indol-3-yl)(5-methoxy-1H-indol-2-yl)methanone

To a mixture of 5-triflyloxy-(1-(chloromethyl)-1,2-dihydro-3H-benzo[e]indol-3-yl)(5-methoxy-1H-indol-2-yl)methanone (27 mg, 0.05 mmol), potassium fluoride (10.5 mg, 0.18 mmol), BINAP (6 mg, 0.01 mmol), and the appropriate substituted phenylboronic acid (0.15 mmol) in dry toluene (1.5 mL) was added Pd(OAc)₂ (1.1 mg, 0.005 mmol) in an oven-dried pressure tube under nitrogen. The reaction mixture was stirred and refluxed at 100° C. for 1 h in oil bath. The crude reaction mixture was applied directly to a silica gel column to afford the purified product.

### Example 6—DP-36

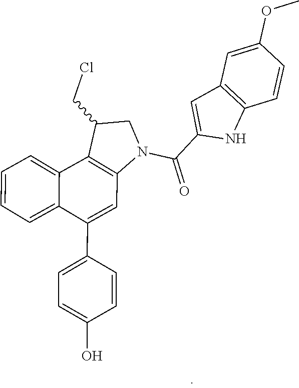 [SMILES: COc1ccc2[nH]c(C(=O)N3CC(CCl)c4c3cc(-c3ccc(O)cc3)c3ccccc43)cc2c1]

HPLC-MS: m/z (%)=483 [M+H], 485 [M+2+H]

### Example 7—DP-44

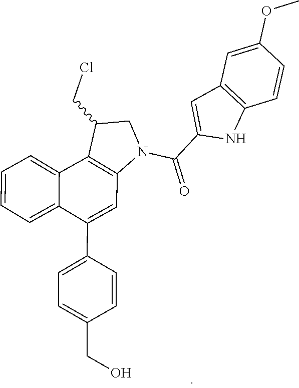 [SMILES: COc1ccc2[nH]c(C(=O)N3CC(CCl)c4c3cc(-c3ccc(CO)cc3)c3ccccc43)cc2c1]

HPLC-MS: m/z (%)=497 [M+H], 499 [M+2+H]

### Example 8—DP-45

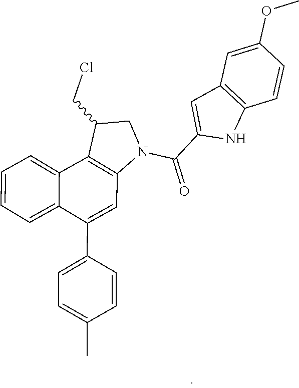 [SMILES: COc1ccc2[nH]c(C(=O)N3CC(CCl)c4c3cc(-c3ccc(C)cc3)c3ccccc43)cc2c1]

HPLC-MS: m/z (%6)=481 [M+H], 483 [M+2+H]

### Example 9—DP-47

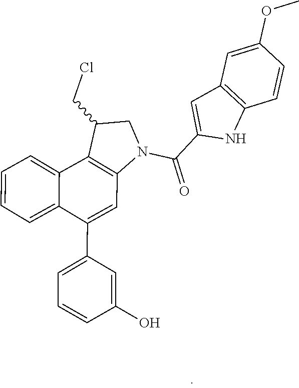 [SMILES: COc1ccc2[nH]c(C(=O)N3CC(CCl)c4c3cc(-c3cccc(O)c3)c3ccccc43)cc2c1]

HPLC-MS: m/z (%)=483 [M+H], 485 [M+2+H]

### Example 10—DP-48

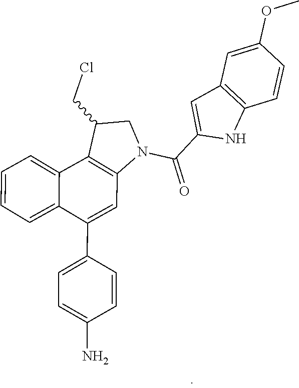 [SMILES: COc1ccc2[nH]c(C(=O)N3CC(CCl)c4c3cc(-c3ccc(N)cc3)c3ccccc43)cc2c1]

HPLC-MS: m/z (%)=482 [M+H], 484 [M+2+H]

### General Synthesis C:

General Method for Ester Synthesis with Alkynoic Acids

To a mixture of (5-(benzyloxy)-1-(chloromethyl)-1,2-dihydro-3H-benzo[e]indol-3-yl)(5-hydroxy-1H-indol-2-yl)methanone (48 mg, 0.1 mmol), triethylamine (42 uL, 0.3 mmol), 1-Ethyl-3-(3-dimethylaminopropyl)carbodiimide hydrochloride (38 mg, 0.2 mmol), in dry dichloromethane (3 mL) was added the appropriate alkynoic acid (0.15 mmol) in an oven-dried flask. The reaction mixture was stirred at room temperature for 15 minutes. The crude reaction mixture was applied directly to a silica gel column to afford the purified product.

### Example 11—DP-42

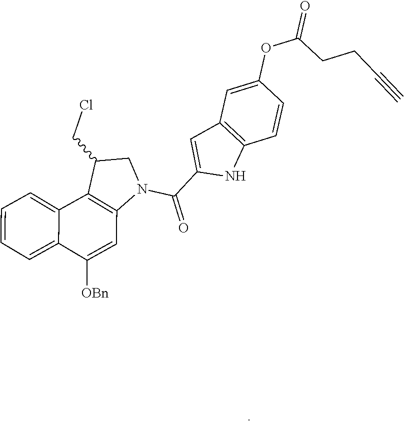 [SMILES: C#CCCC(=O)Oc1ccc2[nH]c(C(=O)N3CC(CCl)c4c3cc(OCc3ccccc3)c3ccccc43)cc2c1]

HPLC-MS: m/z (%)=563 [M+H], 565 [M+2+H]

1H NMR (CDCl₃) δ: 9.82 (s, 1H), 8.36 (dd, J=8/0.8 Hz, 1H), 8.20 (s, 1H), 7.70 (d, J=8 Hz, 1H), 7.58-7.51 (m, 3H), 7.46-7.40 (m, 5H), 7.37-7.33 (m, 1H), 7.07 (d, J=1.4 Hz 1H), 7.03 (dd, J=8.8/2.4 Hz, 1H), 5.28 (m, 2H), 4.79 (dd, J=8/0.8 Hz, 1H), 4.64 (m, 1H), 4.11 (m, 1H), 3.97 (dd, J=8.4/2.4 Hz 1H), 3.47 (t, J=10.8 Hz, 1H), 2.85 (m, 2H), 2.68 (m, 2H), 2.07 (t, J=2.8 Hz, 1H).

### Example 12—DP-51

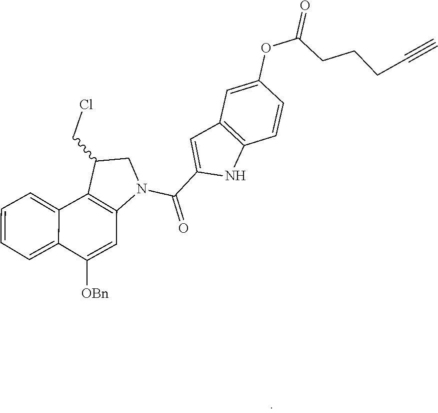 [SMILES: C#CCCCC(=O)Oc1ccc2[nH]c(C(=O)N3CC(CCl)c4c3cc(OCc3ccccc3)c3ccccc43)cc2c1]

HPLC-MS: m/z (%)=577 [M+H], 579 [M+2+H]

### Example 13—DP-77

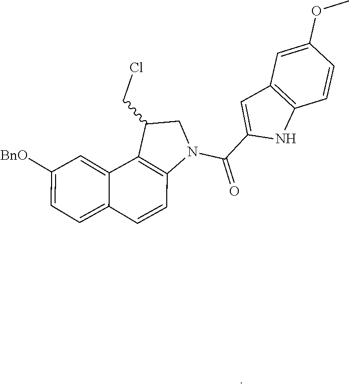 [SMILES: COc1ccc2[nH]c(C(=O)N3CC(CCl)c4c3ccc3ccc(OCc5ccccc5)cc43)cc2c1]

HPLC-MS: m/z (%)=497 [M+H], 499 [M+2+H]

### Example 14—DP-78

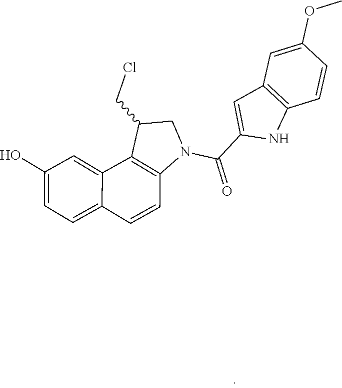 [SMILES: COc1ccc2[nH]c(C(=O)N3CC(CCl)c4c3ccc3ccc(O)cc43)cc2c1]

HPLC-MS: m/z (%)=407 [M+H], 409 [M+2+H]

1H NMR (CDCl₃) δ: 11.64 (s, 1H), 9.87 (s, 1H), 8.17 (d, J=0.8 Hz, 1H), 7.78 (m, 2H), 7.40 (d, J=8.8 Hz, 1H), 7.16-7.10 (m, 3H), 7.02 (dd, J=8.8/1.2 Hz, 1H), 6.92 (dd, J=8.8/2.0 Hz, 1H), 4.80 (t, J=10 Hz, 1H), 4.58 (d, J=10.4 Hz 1H), 4.21 (m, 1H), 4.04 (dd, J=10/2.4 Hz, 1H), 3.88 (dd, J=10.8/7.6 Hz, 1H), 3.78 (s, 3H).

### Example 15—DP-31

Synthesis of Example 15 and Example 16 is based on known methods disclosed in Boger et al. J. Org. Chem. 1999, 64(14), 5241-5244 and Sheldrake et al. J. Med. Chem., 2013, 56(15), 6273-7.

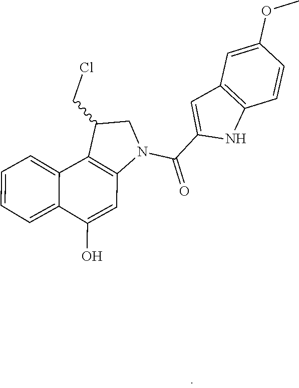 [SMILES: COc1ccc2[nH]c(C(=O)N3CC(CCl)c4c3cc(O)c3ccccc43)cc2c1]

HPLC-MS: m/z (%)=407 [M+H], 409 [M+2+H]

### Example 16—DP-40

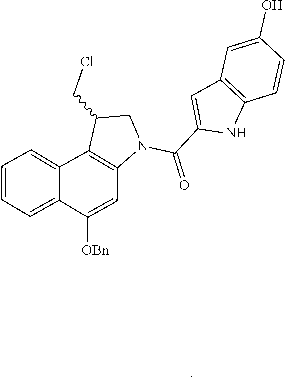 [SMILES: O=C(c1cc2cc(O)ccc2[nH]1)N1CC(CCl)c2c1cc(OCc1ccccc1)c1ccccc21]

HPLC-MS: m/z (%)=483 [M+H], 485 [M+2+H]

### Example 17—tert-Butyl 1-(chloromethyl)-5-phenyl-1H-benzo[e]indole-3(2H)-carboxylate (ICT-11081)

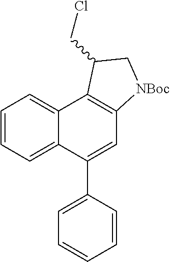 [SMILES: CC(C)(C)OC(=O)N1CC(CCl)c2c1cc(-c1ccccc1)c1ccccc21]

Synthesized with Method B

¹H NMR (400 MHz, CDCl₃) δ 8.16 (bs, 1H), 7.85 (d, 1H, J=8.4 Hz), 7.76 (d, 1H, J=8.0 Hz), 7.51 (ddd, 1H, J=1.2 Hz, J=6.8 Hz, J=8.0 Hz), 7.47-7.39 (m, 5H) 7.26 (ddd, 1H, J=6.8 Hz, J=1.6 Hz, J=8.4 Hz), 4.31 (d, 1H, J=11.4 Hz), 4.15 (dd, 1H, J=8.8 Hz, J=11.4 Hz), 4.09 (m, 1H), 4.00 (dd, 1H, J=2.4H, J=11.2 Hz), 3.52 (dd, 1H, J=10.4 Hz, J=11.2 Hz), 1.57 (s, 9H); ¹³C NMR (100 MHz, CDCl₃) δ 152.61, 140.67, 130.00 (2C), 128.19 (2C), 127.49, 127.38 (2C), 126.96 (2C), 123.94, 122.31, 116.90, 67.09, 53.43, 52.73, 46.32, 28.44 (3C); HRMS (ESI+) Found 394.08164 cacld. For C₂₄H₂₅ClNO₂ 394.15738 [M+H]⁺.

### Example 18—tert-Butyl 1-(chloromethyl)-5-(4-hydroxyphenyl)-1H-benzo[e]indole-3(2H)-carboxylate (ICT-11071)

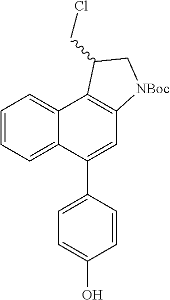 [SMILES: CC(C)(C)OC(=O)N1CC(CCl)c2c1cc(-c1ccc(O)cc1)c1ccccc21]

Synthesized with method B

¹H NMR (400 MHz, CDCl₃) δ 8.17 (bs, 1H), 7.87 (d, 1H, J=8.4 Hz), 7.76 (d, 1H, J=8.0 Hz), 7.50 (ddd, 1H, J=1.2 Hz, J=6.8 Hz, J=8.0 Hz), 7.33 (d, 2H, J=8.6 Hz), 7.30 (ddd, 1H, J=1.2 Hz, J=6.8 Hz, J=8.4 Hz), 6.93 (d, 2H, J=8.6 Hz), 4.98 (bs, 1H), 4.32 (d, 1H, J=11.6 Hz), 4.16 (dd, 1H, J=11.6 Hz, J=8.4 Hz), 4.07 (m, 1H), 4.00 (dd, 1H, J=2.8 Hz, J=11.6 Hz), 3.50 (dd, 1H, J=10.4 Hz, J=11.6 Hz), 1.58 (s, 9H); ¹³C NMR (100 MHz, CDCl₃) δ 154.45, 152.64, 130.97 (2C), 127.42, 126.71, 123.93, 116.59, 115.24 (2C), 55.96, 46.33, 35.48, 20.47 (3C); HRMS (ESI+) Found 410.15188 calcd. For C₂₄H₂₅ClNO₃ 410.15230 [M+H]⁺.

### Example 19—tert-Butyl 1-(chloromethyl)-5-(4-methoxyphenyl)-1H-benzo[e]indole-3(2H)-carboxylate (ICT-11082)

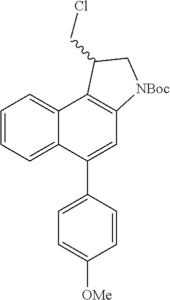 [SMILES: COc1ccc(-c2cc3c(c4ccccc24)C(CCl)CN3C(=O)OC(C)(C)C)cc1]

Synthesized with Method B

¹H NMR (400 MHz, CDCl₃) δ 7.89 (d, 1H, J=8.5 HZ), 7.76 (d, 1H, J=8.3 Hz), 7.48 (dd, 1H, J=6.8 Hz, J=8.3 Hz), 7.40 (d, 2H, J=8.8 Hz), 7.30 (dd, 1H, J=6.8 Hz, J=8.5 Hz), 7.01 (d, 2H, J=8.8 Hz), 6.96 (dt, J=8.78, 1H), 4.32 (d, 1H, J=11.0 Hz), 4.16 (dd, 1H, J=8.8 Hz, J=11.0 Hz), 4.07 (m, 1H), 4.00 (dd, 1H, J=2.6 Hz, J=10.9 Hz), 3.89 (s, 3H), 3.49 (dd, 1H, J=6.3 Hz, J=10.9 Hz), 1.58 (s, 9H). ¹³C NMR (101 MHz, CDCl₃) δ 131.27 (s, 2C), 127.88 (s), 127.69 (s), 123.98 (s), 122.43 (s), 114.31 (s), 113.80 (s, 2C), 55.50 (s), 52.88 (s), 46.46 (s), 42.22, 28.60 (s, 3C);

### Example 20—(1-(Chloromethyl)-5-phenyl-1H-benzo[e]indol-3(2H)-yl)(5-methoxy-1H-indol-2-yl)methanone (ICT-11043)

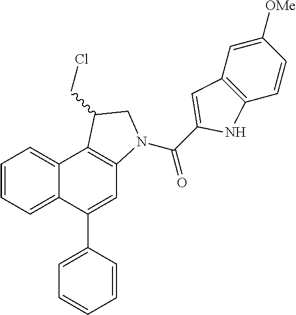 [SMILES: COc1ccc2[nH]c(C(=O)N3CC(CCl)c4c3cc(-c3ccccc3)c3ccccc43)cc2c1]

Synthesized with Method B

¹H NMR (400 MHz, CDCl₃) δ 9.52 (s, 1H, NH), 8.54 (s, 1H), 7.89 (d, 1H, J=8.4 Hz), 7.86 (d, 1H, J=8.0 Hz), 7.56 (ddd, 1H J=1.2 Hz, J=6.8 Hz, J=8.0 Hz), 7.51-742 (m, 5H), 7.37 (ddd, 1H, J=1.2 Hz, J=6.8 Hz, J=8.4 Hz), 7.36 (d, 1H, J=9.2 Hz), 7.13 (d, 1H, J=2.4 Hz), 7.07 (dd, 1H, J=0.8 Hz, J=2.0 Hz), 6.98 (dd, 1H, J=2.4 Hz, J=9.2 Hz), 4.89 (dd, 1H, J=2.0 Hz, J=10.8 Hz), 4.73 (dd, 1H, J=8.4 Hz, J=10.8 Hz), 4.27 (m, 1H), 4.05 (dd, 1H, J=2.8 Hz, J=11.2 Hz), 3.88 (s, 3H), 3.56 (dd, 1H, J=10.5 Hz, J=11.2 Hz); ¹³C NMR (100 MHz, CDCl₃) δ 160.67, 154.74, 142.59, 141.17, 140.43, 131.35, 130.49, 130.12 (2C), 129.81, 129.73, 128.30 (2C), 127.64, 127.54, 127.18, 124.89, 123.94, 122.68, 118.79, 116.84, 115.31, 112.75, 106.07, 102.47, 55.75, 55.05, 45.87, 43.69; HRMS (ESI+) Found 467.15180 calcd. for C₂₉H₂₄ClN₂O₂ 467.15263 [M+H]⁺.

### Example 21—(1-(Chloromethyl)-5-(2-hydroxyphenyl)-1H-benzo[e]indol-3(2H)-yl)(5-methoxy-1H-indol-2-yl)methanone (ICT-11044)

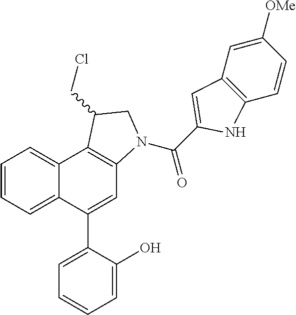 [SMILES: COc1ccc2[nH]c(C(=O)N3CC(CCl)c4c3cc(-c3ccccc3O)c3ccccc43)cc2c1]

Synthesized with Method B

¹H NMR (400 MHz, CDCl₃) δ 9.29 (s, 1H, NH), 8.59 (s, 1H), 7.80 (d, 1H, J=8.4 Hz), 7.62 (dd, 1H, J=4.0 Hz, J=8.4 Hz), 7.52 (m, 1H), 7.36-7.28 (m, 3H), 7.28 (dd, 1H, J=1.2 Hz, J=7.2 Hz), 7.16 (dd, 1H, J=1.6 Hz, J=7.6 Hz), 7.07 (d, 1H, J=2.4 Hz), 7.01-6.93 (m, 4H), 4.84 (dd, J=2.0 Hz, J=10.8 Hz), 4.69 (dd, 1H, J=6.4 Hz, J=10.8 Hz), 4.29 (m, 1H), 4.07 (dd, 1H, J=3.2 Hz, J=11.6 Hz), 3.70 (s, 3H), 3.59 (dd, 1H, J=10.8 Hz, J=11.6 Hz); HRMS (ESI+) Found 483.14694 calcd. for C₂₉H₂₄ClO₃N₂ 483.14755 [M+H]⁺.

### Example 22—(1-(Chloromethyl)-5-(4-methoxyphenyl)-1H-benzo[e]indol-3(2H)-yl)(5-methoxy-1H-indol-2-yl)methanone (ICT-11038)

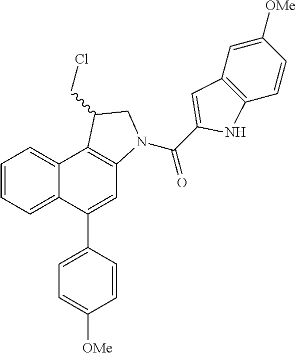 [SMILES: COc1ccc(-c2cc3c(c4ccccc24)C(CCl)CN3C(=O)c2cc3cc(OC)ccc3[nH]2)cc1]

Synthesized with Method B

¹H NMR (400 MHz, CDCl₃) δ 9.29 (s, 1H, NH), 8.50 (s, 1H), 7.94 (d, 1H, J=8.2 Hz), 7.84 (d, 1H, J=8.2 Hz), 7.56 (m, 1H), 7.43 (m, 2H), 7.37 (m, 2H), 7.14 (d, 1H, J=2.2 Hz), 7.07 (dd, 1H, J=2.2 Hz, J=0.4 Hz), 7.02 (m, 3H), 4.89 (dd, 1H, J=10.8 Hz, J=1.6 Hz), 4.74 (m, 1H), 4.26 (m, 1H) 4.06 (dd, 1H, J=11.3 Hz, J=3.1 Hz), 3.9 (s, 3H), 3.88 (s, 3H), 3.554 (dd, 1H, J=10.8 Hz, J=11.3 Hz); ¹³C NMR (100 MHz, CDCl₃) δ 160.52, 159.17, 154.77, 143.94, 142.31, 141.21, 132.81, 131.22 (2C), 130.55, 129.92, 129.83, 128.37, 127.69, 127.12, 124.78, 123.57, 122.66, 118.75, 116.86, 113.74, 112.71, 106.03, 102.53, 55.75, 55.39, 54.97, 45.89, 43.69; HRMS (ESI⁺) found 497.16526 calcd. for C₃₀H₂₆ClN₂O₃ 497.16320 [M+H]⁺.

### Example 23—(1-(Chloromethyl)-5-(4-(methoxymethyl)phenyl)-1H-benzo[e]indol-3(2H)-yl)(5-methoxy-1H-indol-2-yl)methanone (ICT-11039)

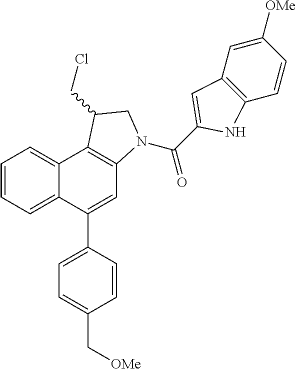 [SMILES: COCc1ccc(-c2cc3c(c4ccccc24)C(CCl)CN3C(=O)c2cc3cc(OC)ccc3[nH]2)cc1]

Synthesized with Method B

¹H NMR (400 MHz, CDCl₃) δ 9.31 (s, 1H, NH), 8.52 (s, 1H), 7.91 (d, 1H, J=8.8 Hz), 7.85 (d, 1H, J=8.0 Hz), 7.56 (m, 1H), 7.4815 (m, 4H), 7.36 (d, 2H, J=8.8 Hz), 7.14 (d, 1H, J=2.4 Hz), 7.07 (m, 1H), 7.01 (dd, 1H, J=8.8 Hz, J=2.4 Hz), 4.89 (dd, 1H, J=10.8 Hz, J=2.0 Hz), 4.74 (m, 1H), 4.56 (s, 2H), 4.27 (m, 1H), 4.06 (dd, 1H, J=11.2 Hz, J=2.8 Hz), 3.88 (s, 3H), 3.56 dd, 1H, J=10.8 Hz, J=11.2 Hz), 3.47 (s, 3H); ¹³C NMR (100 MHz, CDCl₃) δ 160.88, 155.11, 142.67, 141.53, 140.17, 137.88, 131.57, 130.84, 130.53 (2C), 130.14, 130.07, 128.70, 128.04 (2C), 127.97, 127.52, 125.51, 124.23, 123.00, 119.11, 117.21, 113.05, 106.38, 102.86, 74.89, 58.63, 56.08, 55.30, 46.22, 44.03; HRMS (ESI) found 511.17814 calcd. for C₃₁H₂₈ClN₂O₃ 511.17885 [M+H]⁺.

### Example 24—(1-(Chloromethyl)-5-(pyridin-4-yl)-1H-benzo[e]indol-3(2H)-yl)(5-methoxy-1H-indol-2-yl)methanone (ICT-11040)

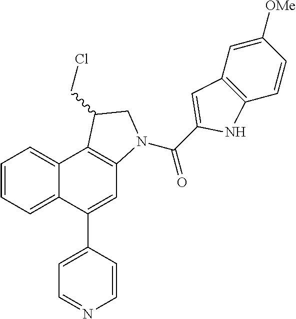 [SMILES: COc1ccc2[nH]c(C(=O)N3CC(CCl)c4c3cc(-c3ccncc3)c3ccccc43)cc2c1]

Synthesized with Method B

¹H NMR (400 MHz, CDCl₃) δ 9.36 (s, 1H, NH), 8.75 (dd, 2H, J=4.4 Hz, J=1.6 Hz), 8.54 (s, 1H), 7.86 (d, 2H, J=8.0 Hz), 7.61 (dd, 1H, J=7.8 Hz, J=1.2 Hz), 7.45 (d, 2H, J=6 Hz), 7.42 (m, 1H), 7.36 (d, 1H, J=8.8 Hz), 7.14 (d, 1H, J=2.4 Hz), 7.08 (dd, 1H, J=2 Hz, J=0.8 Hz), 7.01 (dd, 1H, J=8.8 Hz, J=2.4 Hz), 4.91 (dd, 1H, J=10.8 Hz, J=2.0 Hz), 4.75 (m, 1H), 4.29 (m, 1H), 4.06 (m, 1H), 3.87 (s, 3H), 3.58 (dd, 1H, J=10.4 Hz, J=10.8 Hz); ¹³C NMR (100 MHz, CDCl₃) δ 161.0, 155.1, 150.2, 148.8, 141.5, 139.7, 131.6, 130.5, 130.2, 129.1, 128.6, 127.9, 127.1, 125.8, 125.4, 123.0, 119.1, 117.3, 113.0, 106.5, 102.8, 56.0, 55.2, 45.1, 43.9; HRMS (ES+I) Found 468.14703 calcd. for C₂₈H₂₃ClN₃O₂ 468.14778 [M+H]⁺.

### Example 25—(1-(Chloromethyl)-5-(pyridin-3-yl)-1H-benzo[e]indol-3(2H)-yl)(5-methoxy-1H-indol-2-yl)methanone (ICT-11041)

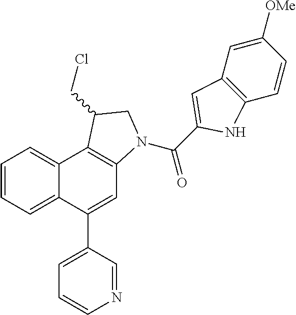 [SMILES: COc1ccc2[nH]c(C(=O)N3CC(CCl)c4c3cc(-c3cccnc3)c3ccccc43)cc2c1]

Synthesized with Method B

¹H NMR (400 MHz, CDCl₃) δ 9.51 (s, 1H, NH), 8.78 (d, 1H, J=1.6 Hz), 8.72 (dd, 1H, J=1.6 Hz, J=4.9 Hz), 8.55 (s, 1H), 7.88 (d, 1H, J=8.6 Hz), 7.85 (m, 1H), 7.81 (d, 1H, J=8.4 Hz), 7.6 (ddd, 1H, J=1.1 Hz, J=6.8 Hz, J=8.4 Hz), 7.46 (ddd, 1H, J=2.1 Hz, J=4.5 Hz, J=6.3 Hz), 7.43 (dd, 1H, J=1.3 Hz, J=7.0 Hz), 7.41 (dd, 1H, J=1.3 Hz, J=6.9 Hz), 7.36 (d, 1H, J=8.9 Hz), 7.14 (d, 1H, J=2.4 Hz), 7.08 (d, 1H, J=2.0 Hz), 7.00 (dd, 1H, J=2.4 Hz, J=8.9 Hz), 4.91 (dd, 1H, J=1.9 Hz, J=10.8 Hz), 4.76 (dd, 1H, J=8.7 Hz, J=10.8 Hz), 4.29 (m, 1H), 4.06 (dd, 1H, J=2.7 Hz, J=11.3 Hz), 3.88 (s, 3H) 3.58 (dd, 1H, J=10.5 Hz, J=11.3 Hz); ¹³C NMR (100 MHz, CDCl₃) δ 160.75, 154.80, 150.72, 148.92, 137.98, 131.40, 128.30, 127.52, 126.88, 125.43, 124.89, 123.37, 122.90, 119.32, 117.01, 112/79, 106.23, 102.84, 55.99, 55.29, 46.11, 44.04; HRMS (ESI+) Found 468.14988 calcd. for C₂₈H₂₃ClO₂N₃ 468.14788 [M+H]+.

### Example 26—(1-(Chloromethyl)-5-(4-(dimethylamino)phenyl)-1H-benzo[e]indol-3(2H)-yl)(5-methoxy-1H-indol-2-yl)methanone (ICT-11042)

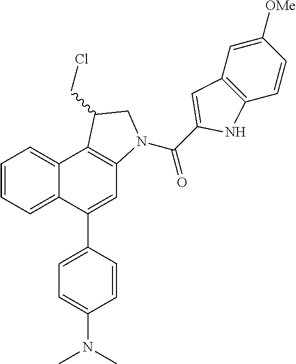 [SMILES: COc1ccc2[nH]c(C(=O)N3CC(CCl)c4c3cc(-c3ccc(N(C)C)cc3)c3ccccc43)cc2c1]

Synthesized with Method B

¹H NMR (400 MHz, CDCl₃) δ 9.34 (s, 1H, NH), 8.50 (s, 1H), 8.04 (d, 1H, J=8.0 Hz), 7.85 (d, 1H, J=8.4 Hz), 7.54 (ddd, 1H, J=1.2 Hz, J=6.8 Hz, J=8.4 Hz), 7.42 (d, 2H, J=8.8 Hz), 7.39 (ddd, 1H, J=1.2 Hz, J=6.8 Hz, J=8.0 Hz), 7.37 (d, 1H, J=8.9 Hz), 7.14 (d, 1H, J=2.4 Hz), 7.06 (d, 1H, J=1.9 Hz), 7.00 (dd, 1H, J=2.4 Hz, J=8.9 Hz), 6.85 (d, 2H, J=8.8 Hz), 4.88 (dd, 1H, J=1.7 Hz, J=10.8 Hz), 4.72 (dd, J=8.7 Hz, J=10.8 Hz), 4.24 (m, 1H), 4.06 (dd, 1H, J=3.1 Hz, J=11.1 Hz), 3.88 (s, 3H), 3.54 (dd, 1H, J=10.4 Hz, J=11.1 Hz), 2.04 (s, 6H); ¹³C NMR (100 MHz, CDCl₃) δ 160.50, 154.74, 149.99, 142.98, 141.28, 131.22, 130.94 (2C), 130.66, 130.02, 129.91, 128.40, 127.97, 127.99, 124.55, 123.05, 122.59, 118.52, 116.77, 112/71, 112.20 92C), 105.95, 102.51, 56.04, 55.13, 46.91, 43.83, 41.36, 40.61; HRMS (ESI+) Found 510.19404 calcd. for C₃₁H₂₉ClO₂N₃ 510.19483 [M+H]⁺.

### Example 27—(5-(6-Aminopyridin-3-yl)-1-(chloromethyl)-1H-benzo[e]indol-3(2H)-yl)(5-methoxy-1H-indol-2-yl)methanone (ICT-11083)

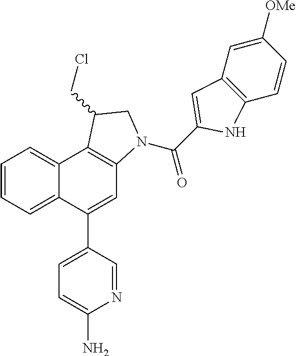 [SMILES: COc1ccc2[nH]c(C(=O)N3CC(CCl)c4c3cc(-c3ccc(N)nc3)c3ccccc43)cc2c1]

Synthesized with Method B

¹H NMR (400 MHz, CDCl₃) δ 9.29 (s, 1H, NH), 8.48 (s, 1H), 8.24 (s, 1H), 7.90 (d, 1H, J=8.4 Hz), 7.84 (d, 1H, J=8.4 Hz), 7.62 (dd, 1H, J=8.6 Hz, J=2.4 Hz), 7.57 (m, 1H), 7.55 (m, 1H), 7.42 (d, 1H, J=1.2 Hz), 7.39 (m, 1H), 7.14 (d, 1H, J=2.4 Hz), 7.07 (m, 1H), 7.03 (m, 1H), 6.67 (d, 1H, J=8.4 Hz), 4.89 (m, 1H), 4.73 (m, 1H), 4.26 (m, 1H), 4.05 (dd, 1H, J=11.2 Hz, J=3.2 Hz), 3.88 (s, 3H), 3.55 (dd, 1H, J=10.8 Hz, J=11.2 Hz);

### Example 28—(1-(Chloromethyl)-5-(3-fluoro-4-hydroxyphenyl)-1,2-dihydro-3H-benzo[e]indol-3-yl)(5-methoxy-1H-indol-2-yl)methanone (ICT-11075)

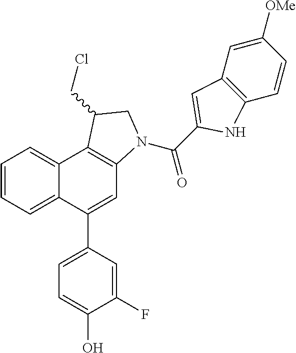 [SMILES: COc1ccc2[nH]c(C(=O)N3CC(CCl)c4c3cc(-c3ccc(O)c(F)c3)c3ccccc43)cc2c1]

Synthesized with Method B

¹H NMR (400 MHz, acetone-d₆) δ 10.79 (s, 1H, NH), 8.88 (s, 1H), 8.51 (s, 1H), 8.05 (d, 1H, J=8.0 Hz), 7.9 (d, 1H, J=8.4 Hz), 7.59 (ddd, 1H, J=1.2 Hz, J=6.8 Hz, J=8.0 Hz), 7.47 (d, 1H, J=8.8 Hz), 7.42 (ddd, 1H, J=1.2 Hz, J=6.8 Hz, J=8.4 Hz), 7.26 (m, 1H), 7.18 (m, 4H), 6.94 (dd, 1H, J=2.4 Hz, J=8.8 Hz), 4.86 (m, 2H), 4.45 (m, 1H), 4.15 (dd, 1H, J=3.4 Hz, J=11.2 Hz), 3.94 (dd, 1H, J=8.4 Hz, J=11.2 Hz), 3.83 (s, 3H, OMe); ¹³C NMR (100 MHz, acetone-d₆) δ 161.3, 155.5, 142.7, 141.2, 133.5 (2C), 132.7, 132.0, 131.0, 130.2, 129.2, 127.9, 127.7, 127.1 (2C), 125.6, 125.3, 124.1, 119.6, 118.6, 118.4, 118.2, 116.9, 113.9, 106.5, 103.0, 55.7, 47.6, 43.5;

### Example 29—4-(1-(Chloromethyl)-3-(5-methoxy-1H-indole-2-carbonyl)-2,3-dihydro-1H-cyclopenta[a]naphthalen-5-yl)phenyl acetate (ICT-11072)

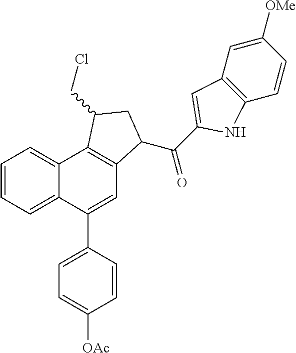 [SMILES: COc1ccc2[nH]c(C(=O)C3CC(CCl)c4c3cc(-c3ccc(OC(C)=O)cc3)c3ccccc43)cc2c1]

Synthesized with Method B

¹H NMR (400 MHz, acetone-d₆) δ 10.79 (s, 1H, NH), 8.53 (s, 1H), 8.06 (d, 1H, J=8.4 Hz), 7.84 (d, 1H, J=8.4 Hz), 7.59 (dd, 1H, J=7.6 Hz, J=8.4 Hz), 7.54 (d, 2H, J=8.4 Hz), 7.47 (d, 1H, J=8.8 Hz), 7.42 (dd, 1H, J=7.6 Hz, J=8.4 Hz), 7.30 (d, 2H, J=8.4 Hz), 7.19 (s, 2H), 6.94 (dd, 1H, J=2.6 Hz, J=8.8 Hz), 4.98 (m, 2H), 4.45 (m, 1H), 4.16 (dd, 1H, J=3.2 Hz, J=11.2 Hz), 3.95 (dd, 1H, J=8.4 Hz, J=11.2 Hz), 3.83 (s, 3H), 2.31 (s, 3H); ¹³C NMR (100 MHz, acetone-d₆) δ 169.7, 161.3, 155.5, 151.5, 142.7, 141.6, 139.0, 132.7, 132.0, 131.7, 131.0, 130.1, 129.2, 127.9, 127.6, 125.7, 125.5, 124.2, 123.3, 122.7, 119.6, 116.9, 113.9, 106.5, 103.0, 55.7, 47.6, 43.5, 21.0;

### Example 30—(1-(Chloromethyl)-5-(4-hydroxy-3-nitrophenyl)-2,3-dihydro-1H-cyclopenta[a]naphthalen-3-yl)(5-methoxy-1H-indol-2-yl)methanone (ICT-11069)

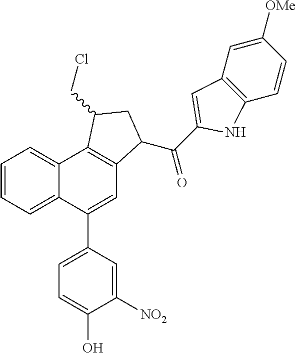 [SMILES: COc1ccc2[nH]c(C(=O)C3CC(CCl)c4c3cc(-c3ccc(O)c([N+](=O)[O-])c3)c3ccccc43)cc2c1]

Synthesized with Method B

¹H NMR (400 MHz, THF-d₈) δ 10.93 (s, 1H, NH), 10.56 (s, 1H), 8.58 (s, 1H), 8.21 (d, 1H, J=2.4 Hz), 8.0 (d, 1H, J=8.4 Hz), 7.79 (d, 1H, J=8.4 Hz), 7.76 (dd, 1H, J=2.2 Hz, J=8.6 Hz), 7.56 (ddd, 1H, J=1.2 Hz, J=6.8 Hz, J=8.4 Hz), 7.37 (ddd, 1H, J=1.6 Hz, J=6.8 Hz, J=8.4 Hz), 7.34 (d, 1H, J=9.2 Hz), 7.3 (d, 1H, J=8.8 Hz), 7.11 (m, 2H), 6.9 (dd, 1H, J=2.4 Hz, J=9.2 Hz), 4.85 (m, 2H), 4.36 (m, 1H), 4.09 (dd, 1H, J=3.2 Hz, J=11.2 Hz), 3.8 (s, 3H), 3.74 (dd, 1H, J=9.4 Hz, J=11.2 Hz); ¹³C NMR (100 MHz, THF-d₈) δ 161.4, 155.7, 142.9, 139.9, 139.4, 135.2, 133.8, 133.0, 132.0, 131.2, 130.1, 129.3, 127.9, 127.3, 126.8, 125.8, 125.7, 124.1, 121.0, 120.0, 116.9, 113.5, 106.3, 102.8, 55.9, 55.6, 47.1, 44.2;

### Example 31—(1-(Chloromethyl)-5-(4-hydroxy-2-methylphenyl)-2,3-dihydro-1H-cyclopenta[a]naphthalen-3-yl)(5-methoxy-1H-indol-2-yl)methanone (ICT-11070)

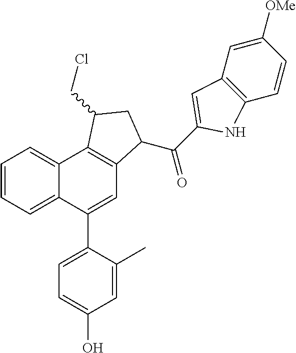 [SMILES: COc1ccc2[nH]c(C(=O)C3CC(CCl)c4c3cc(-c3ccc(O)cc3C)c3ccccc43)cc2c1]

Synthesized with Method B

¹H NMR (400 MHz, acetone-d₆) δ 10.77 (s, 1H, NH), 8.42 (bs, 1H), 8.41 (d, 1H, J=1.6 Hz), 8.04 (dd, 1H, J=0.8 Hz, J=8.4 Hz), 7.56 (m, 1H), 7.46-7.5 (m, 2H), 7.36 (ddd, 1H, J=1.2 Hz, J=6.8 Hz, J=8.4 Hz), 7.19 (m, 2H), 7.07 (m, 1H), 6.94 (dd, 1H, J=2.4 Hz, J=9.2 Hz), 6.87 (dd, 1H, J=2.6 Hz, J=4.8 Hz), 6.82 (td, 1H, J=2.6 Hz, J=8.6 Hz, H6″), 4.91 (m, 2H), 4.47 (m, 1H), 4.18 (m, 1H), 3.95 (ddd, 1H, J=3.6 Hz, J=8.4 Hz, J=11.2 Hz); 3.83 (s, 3H), 1.99 (s, 3H, Me); ¹³C NMR (100 MHz, acetone-d₆) δ 161.2, 155.5, 146.4, 146.0, 142.1, 141.1, 140.5, 136.1, 132.7, 132.0, 131.6, 130.8, 129.2, 127.9, 127.7, 125.3, 124.9, 124.1, 119.7, 117.5, 116.8, 113.9, 113.5, 106.4, 103.0, 55.7, 47.6, 43.7, 20.3;

### Example 32—(1-(Chloromethyl)-5-(4-hydroxy-3-methylphenyl)-2,3-dihydro-1H-cyclopenta[a]naphthalen-3-yl)(5-methoxy-1H-indol-2-yl)methanone (ICT-11079)

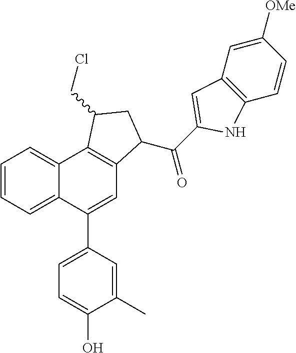 [SMILES: COc1ccc2[nH]c(C(=O)C3CC(CCl)c4c3cc(-c3ccc(O)c(C)c3)c3ccccc43)cc2c1]

Synthesized with Method B

¹H NMR (400 MHz, THF-d₈) δ 10.86 (s, 1H, NH), 8.47 (s, 1H), 8.3 (s, 1H), 7.91 (m, 2H), 7.49 (ddd, 1H, J=1.2 Hz, J=6.8 Hz, J=8.4 Hz), 7.34 (d, 1H, J=8.8 Hz), 7.29 (ddd, 1H, J=1.2 Hz, J=6.8 Hz, J=8.4 Hz), 7.18 (d, 1H, J=2.0 Hz), 7.09 (m, 2H), 7.07 (dd, 1H, J=0.8 Hz, J=2.0 Hz), 6.89 (dd, 1H, J=2.4 Hz, J=8.8 Hz), 6.81 (d, 1H, J=8.4 Hz), 4.81 (m, 2H), 4.29 (m, 1H), 4.07 (dd, 1H, J=2.8 Hz, J=11.2 Hz), 3.79 (s, 3H), 3.68 (dd, 1H, J=9.8 Hz, J=11.2 Hz), 2.25 (s, 3H, Me); ¹³C NMR (100 MHz, THF-d₈) δ 161.3, 156.3, 155.6, 143.3, 142.8, 133.3, 133.0, 132.7, 132.3, 131.1, 130.7, 129.3, 129.1, 128.4, 127.4, 125.1, 124.8, 124.2, 123.7, 119.6, 116.7, 115.0, 113.4, 106.1, 102.9, 55.9, 55.6, 47.0, 44.3, 16.3;

### Example 33—(1-(Chloromethyl)-5-(4-nitrophenyl)-2,3-dihydro-1H-cyclopenta[a]naphthalen-3-yl)(5-methoxy-1H-indol-2-yl)methanone (ICT-11078)

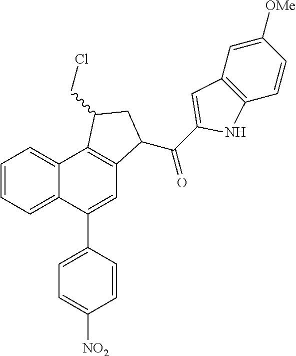 [SMILES: COc1ccc2[nH]c(C(=O)C3CC(CCl)c4c3cc(-c3ccc([N+](=O)[O-])cc3)c3ccccc43)cc2c1]

Synthesized with Method B

¹H NMR (400 MHz, THF-d₈) δ 10.89 (s, 1H, NH), 8.61 (s, 1H), 8.38 (d, 2H, J=8.8 Hz), 8.01 (d, 1H, J=8.2 Hz), 7.75 (m, 3H), 7.57 (ddd, 1H, J=1.2 Hz, J=6.8 Hz, J=8.2 Hz), 7.38 (ddd, 1H, J=1.2 Hz, J=6.8 Hz, J=8.4 Hz), 7.34 (d, 1H, J=8.8 Hz), 7.11 (m, 2H), 6.9 (dd, 1H, J=2.4 Hz, J=8.8 Hz), 4.85 (m, 2H), 4.38 (m, 1H), 4.1 (dd, 1H, J=3.4 Hz, J=11.0 Hz), 3.8 (s, 3H, OMe), 3.76 (dd, 1H, J=9.4 Hz, J=11.0 Hz, CH₂—Cl); ¹³C NMR (100 MHz, THF-d₈) δ 161.6, 155.7, 147.6 (2C), 143.0, 141.3, 140.5, 138.0, 133.1, 132.1, 131.3, 129.9, 128.1, 127.4, 126.4, 125.9, 124.4 (2C), 120.0, 117.0, 113.6, 106.4, 103.0, 56.0, 55.8, 47.2, 44.3;

### Example 34—(1-(Chloromethyl)-5-(4-hydroxyphenyl)-1H-benzo[e]indol-3(2H)-yl)(5-methoxybenzofuran-2-yl)methanone (ICT-11076)

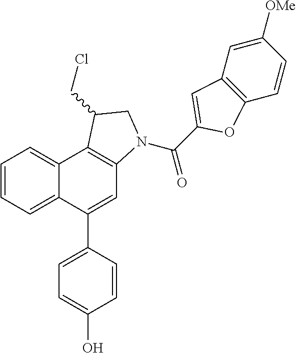 [SMILES: COc1ccc2oc(C(=O)N3CC(CCl)c4c3cc(-c3ccc(O)cc3)c3ccccc43)cc2c1]

Synthesized with Method B

¹H NMR (400 MHz, DMSO-d₆) δ 9.64 (s, 1H, NH), 8.29 (bs, 1H), 8.03 (d, 1H, J=8.0 Hz), 7.84 (d, 1H, J=8.4 Hz), 7.66 (s, 1H), 7.75 (d, 1H, J=9.2 Hz), 7.58 (d, 1H, J=1.2 Hz, J=6.8 Hz, J=8.0 Hz), 7.54 (ddd, 1H, J=1.2 Hz, J=6.8 Hz, J=8.4 Hz), 7.30 (d, 1H, J=2.6 Hz), 7.27 (d, 2H, J=8.4 Hz), 7.10 (dd, 1H, J=2.6 Hz, J=9.2 Hz), 6.92 (d, 2H, J=8.4 Hz), 4.82 (dd, 1H, J=10.4 Hz, J=11.6 Hz), 4.66 (dd, 1H, J=1.6 Hz, J=11.6 Hz), 4.41 (m, 1H), 4.10 (dd, 1H, J=3.2 Hz, J=11.2 Hz), 3.99 (dd, 1H, J=7.2 Hz, J=11.2 Hz), 3.82 (s, 3H); ¹³C NMR (100 MHz, DMSO-d₆) δ 157.27, 157.05, 156.17, 149.31, 149.26, 141.10, 140.87, 130.83 (2C), 130.55, 129.69, 129.07, 127.43, 127.02, 126.70, 124.93, 124.42, 123.60, 117.79, 117.66, 115.32 (2C), 112.80, 112.69, 104.08, 55.64, 54.24, 47.46, 41.49; HRMS (ESI+) Found 484.12993 calcd. for C₂₉H₂₃ClNO₄ 484.13156 [M+H]⁺.

### Example 35—(1-(Chloromethyl)-5-(4-hydroxyphenyl)-1H-benzo[e]indol-3(2H)-yl)(5-methoxybenzo[b]thiophen-2-yl)methanone (ICT-11077)

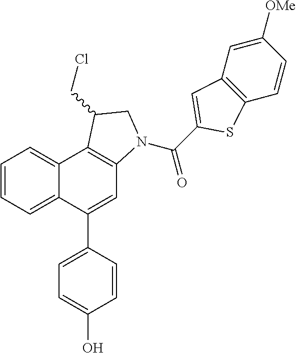 [SMILES: COc1ccc2sc(C(=O)N3CC(CCl)c4c3cc(-c3ccc(O)cc3)c3ccccc43)cc2c1]

Synthesized with Method B

¹H NMR (400 MHz, DMSO-d₆) δ 9.65 (s, 1H, NH), 8.19 (bs, 1H), 8.08 (s, 1H), 8.03 (d, 1H, J=8.2 Hz), 7.94 (d, 1H, J=8.8 Hz), 7.85 (d, 1H, J=8.0 Hz), 7.58 (dd, 1H, J=7.4 Hz, J=8.2 Hz), 7.53 (d, 1H, J=2.4 Hz), 7.43 (dd, 1H, J=7.4 Hz, J=8.0 Hz), 7.26 (d, 2H, J=8.0 Hz), 7.15 (dd, 1H, J=2.4 Hz, J=8.8 Hz), 6.92 (d, 2H, J=8.0 Hz), 4.84 (dd, 1H, J=9.6 Hz, J=10.8 Hz), 4.56 (dd, 1H, J=1.6 Hz, J=10.8 Hz), 4.41 (m, 1H), 4.10 (dd, 1H, J=3.2 Hz, J=11.2 Hz), 4.02 (dd, 1H, J=10.4 Hz, J=11.2 Hz), 3.84 (s, 3H); ¹³C NMR (100 MHz, DMSO-d₈) δ 161.12, 157.48, 157.05, 141.06, 140.91, 140.24, 139.96, 132.40, 130.85 (2C), 130.56, 129.74, 127.03, 126.80, 124.48, 123.61, 123.40, 117.92, 117.26, 115.33 (2C), 106.90, 55.37, 54.92, 47.47, 41.45; HRMS (ESI+) Found 500.10809 calcd. for C₂₉H₂₃ClNO₃S 500.10872 [M+H]⁺.

### Example 36—(1-(Chloromethyl)-5-(4-hydroxyphenyl)-1,2-dihydropyrrolo[3,2-e]indol-3(6H)-yl)(5-methoxy-1H-indol-2-yl)methanone (ICT-11084)

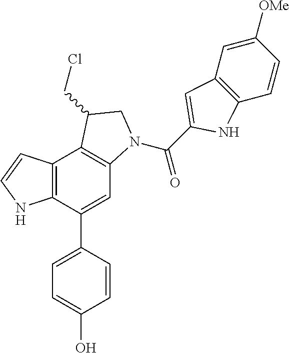 [SMILES: COc1ccc2[nH]c(C(=O)N3CC(CCl)c4c3cc(-c3ccc(O)cc3)c3[nH]ccc43)cc2c1]

Synthesized with Method B

¹H NMR (400 MHz, CDCl₃) δ 9.30 (s, 1H, NH), 8.51 (s, 1H), 8.37 (s, 1H), 7.56 (d, 2H, J=8.4 Hz), 7.40 (d, 1H, J=8.8 Hz), 7.33 (dd, 1H, J=1.6 Hz, J=2.8 Hz), 7.17 (d, 1H, J=2.4 Hz), 7.06-7.02 (m, 4H), 6.73 (s, 1H), 6.59 (dd, 1H, J=2.0 Hz, J=3.2 Hz), 4.77 (m, 2H), 4.21 (m, 1H), 4.16 (dd, 1H, J=4.0 Hz, J=11.2 Hz), 3.90 (s, 3H), 3.68 (dd, 1H, J=10.4 Hz, J=11.2 Hz); HRMS (ESI+) Found 494.12409 calcd. for C₂₇H₂₂ClN₃NaO₃ 494.12474 [M+Na]⁺.

### Example 37—(1-(Chloromethyl)-5-(4-methoxyphenyl)-1,2-dihydropyrrolo[3,2-e]indol-3(6H)-yl)(5-methoxy-1H-indol-2-yl)methanone (ICT-11085)

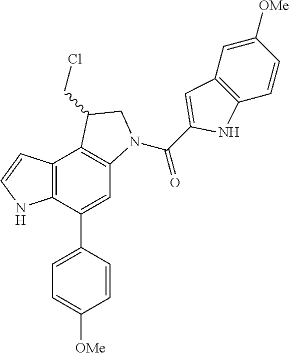 [SMILES: COc1ccc(-c2cc3c(c4cc[nH]c24)C(CCl)CN3C(=O)c2cc3cc(OC)ccc3[nH]2)cc1]

Synthesized with Method B

¹H NMR (400 MHz, CDCl₃) δ 9.41 (s, 1H, NH), 8.51 (s, 1H), 8.36 (s, 1H), 7.60 (d, 2H, J=8.8 Hz), 7.37 (d, 1H, J=8.9 Hz), 7.32 (dd, 1H, J=2.8 Hz, J=3.2 Hz), 7.16 (d, 1H, J=2.4 Hz), 7.06 (d, 2H, J=8.8 Hz), 7.00 (dd, 1H, J=2.4 Hz, J=8.9 Hz), 6.79 (d, 1H, J=1.6 Hz), 6.58 (dd, 1H, J=1.6 Hz, J=3.2 Hz), 4.76 (dd, 1H, J=9.2 Hz, J=10.8 Hz), 4.72 (dd, 1H, J=4.0 Hz, J=10.8 Hz), 4.20 (m, 1H), 4.14 (dd, 1H, J=3.6 Hz, J=10.8 Hz), 3.90 (s, 3H), 3.89 (s, 3H), 3.66 (dd, 1H, J=10.0 Hz, J=10.8 Hz); ¹³C NMR (100 MHz, CDCl₃) δ 159.28, 154.64, 137.66, 132.08, 131.21, 131.16, 131.03, 129.47 (2C), 128.45, 126.08, 125.79, 123.93, 120.13, 116.37, 114.82, 114.64 (2C), 112.96, 112.64, 105.56, 102.52, 100.17, 55.79, 55.42, 54.85, 46.43, 44.10; HRMS (ESI+) Found 486.158833 calcd. For C₂₈H₂₅ClN₃O₃ 486.15844 [M+H]⁺.

### Example 38—(1-(Chloromethyl)-5-((4-methoxyphenyl)(methyl)amino)-1,2-dihydro-3H-benzo[e]indol-3-yl)(5-methoxy-1H-indol-2-yl)methanone (ICT-11067)

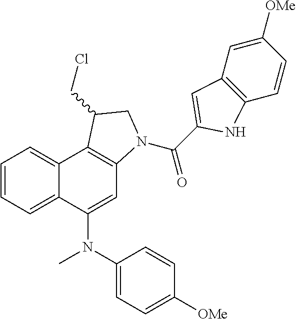 [SMILES: COc1ccc(N(C)c2cc3c(c4ccccc24)C(CCl)CN3C(=O)c2cc3cc(OC)ccc3[nH]2)cc1]

Synthesized with Method A

¹H NMR (400 MHz, acetone-d₈) δ 10.72 (s, 1H, NH), 8.42 (s, 1H), 8.0 (d, 1H, J=8.0 Hz), 7.94 (d, 1H, J=8.4 Hz), 7.56 (ddd, 1H, J=1.6 Hz, J=6.8 Hz, J=8.0 Hz), 7.46 (d, 1H, J=8.8 Hz), 7.36 (ddd, 1H, J=1.2 Hz, J=6.8 Hz, J=8.4 Hz), 7.18 (m, 2H), 6.94 (dd, 1H, J=2.4 Hz, J=8.8 Hz), 6.77 (dd, 2H, J=2.4 Hz, J=6.8 Hz), 6.68 (dd, 2H, J=2.4 Hz, J=6.8 Hz), 4.87 (m, 2H), 4.43 (dd, 1H, J=2.8 Hz, J=8.4 Hz), 4.16 (dd, 1H, J=3.2 Hz, J=11.2 Hz), 3.94 (dd, 1H, J=8.4 Hz, J=11.2 Hz), 3.83 (s, 3H), 3.7 (s, 3H), 3.38 (s, 3H); ¹³C NMR (100 MHz, acetone-d₈) δ 161.2, 155.5, 153.8, 148.7, 145.8, 143.8, 132.7, 132.0, 131.7, 129.2, 128.1, 126.0, 125.4, 124.4, 123.1, 117.6, 116.9, 116.5, 115.2, 113.8, 106.4, 103.0, 55.7, 47.7, 43.4, 41.7;

### Example 39

The compounds of the present invention were evaluated for their ability to inhibit the growth of MDA-MB-231 (triple-negative) and MCF-7 (oestrogen receptor positive) breast cancer cell lines. The compounds displayed antiproliferative activity in the pico- to micromolar range, as shown in Table 1. A positive control (PC), seco-CBI-MI, with structure shown below, was used as a baseline for comparison of the efficacy of the compounds of the present invention. Surprisingly it was found that modifying the phenolic OH, previously thought vital for efficacy, retained cellular potency.

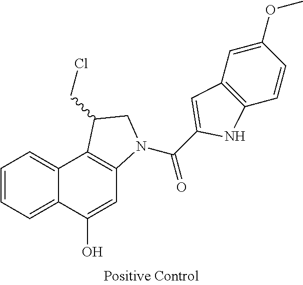 [SMILES: COc1ccc2[nH]c(C(=O)N3CC(CCl)c4c3cc(O)c3ccccc43)cc2c1]

TABLE 1

| | | IC₅₀ | | | |
|---|---|---|---|---|---|
| EX | | MDA MB 231 | | MCF7 | |
| | PC | 0.62 ± 0.5 | nM | 0.69 ± 0.3 | nM |
| 6 | DP36 | 23.29 ± 9.9 | nM | 20.03 ± 27.1 | nM |
| 16 | DP40 | 2.2 ± 0.2 | μM | 1.3 ± 0.1 | μM |
| 11 | DP42 | 3.2 ± 2.9 | μM | tbc | |
| 7 | DP44 | 5.4 ± 5.8 | μM | 4.8 ± 4.8 | μM |
| 8 | DP45 | >10 | μM | >10 | μM |
| 9 | DP47 | 206.3 ± 79.6 | nM | 103.6 ± 79.8 | nM |
| 10 | DP48 | 911 | nM | 713 | nM |
| 12 | DP51 | >10 | μM | tbc | |
| 2 | DP59 | 7.6 ± 2.1 | nM | 2.5 ± 0.7 | nM |
| 3 | DP63 | 1.7 ± 1.4 | nM | 0.8 ± 0.3 | nM |
| 4 | DP65 | 28.3 ± 29 | nM | 8.1 ± 7 | nM |
| 5 | DP70 | 129.4 ± 18.9 | nM | 76.7 ± 29 | nM |
| 13 | DP77 | >10 | μM | >10 | μM |
| 14 | DP78 | 1.08 ± 0.2 | μM | 1.02 ± 0.2 | μM |

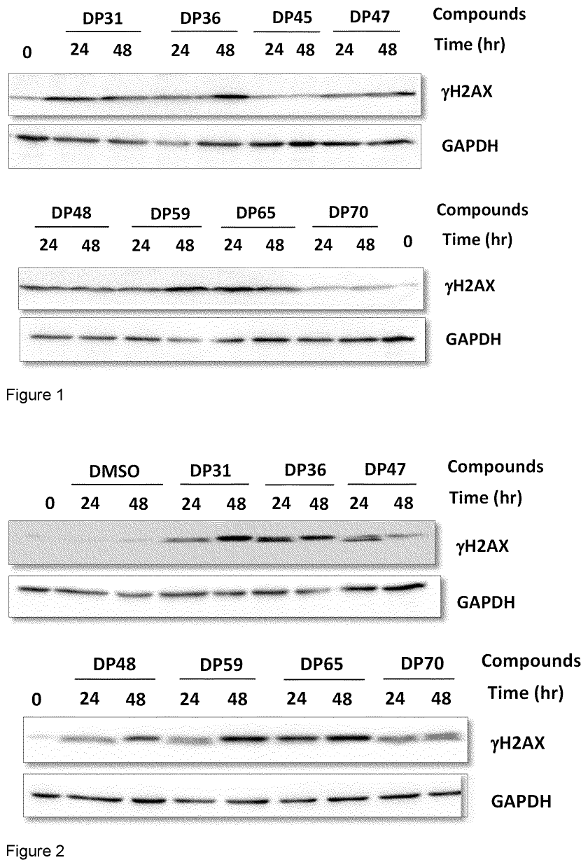

The mechanism of action of the naturally occurring duocarmycins is well documented and involves spirocyclization of the seco-duocamycins including those derived from the CBI and other aryl-ring-fused subunits of the duocarmycin alkylating subunit. Selected compounds (Example 15 (DP-31 (CBI-MI)), Example 6 (DP36), Example 8 (DP45), Example 9 (DP47), Example 8 (DP45), Example 2 (DP59), Example 4 (DP65), Example 5 (DP70)) generated DNA damage as measured using H2AX phosphorylation as a marker of double-stranded DNA damage in a MDA-MB-231 human breast cancer cell line (Western Blot images shown in FIG. 1). This observation suggest that differential cytotoxicity is reflected pharmacodynamically via DNA damage despite the modulation of the phenolic OH position of the CBI alkylating subunit

The expression of γ-H2AX phosphorylations as a marker of DNA damage in MCF-7 human breast cancer cells following treatment with duocarmycin compounds was explored. Breast cancer cells were treated with duocarmycin's of the present invention for 24 or 48 hours (concentration: 10× IC50) or with solvent alone (DMSO). Expression of γ-H2AX was determined by Western blot analysis and is shown in FIG. 2.

The present application provides novel substituents for use within the duocarmycin framework. The examples provided above validate the functionality as duocarmycins of the substituents of the present invention utilising a representative CBI core, the core shown in Formula IA. The substituents of the present invention can be readily applied to other core structures which have proven activity as cancer therapies and that fall within the family of duocarmycins. These cores are represented by the compounds of Formula IB to IL. Evidence for the activity of the cores of compounds of Formula IB to IL can be found in the following review article and the citations contained therein, “Chemical and Biological Explorations of the Family of CC-1065 and the Duocarmycin Natural Products”, Ghosh et al, Current Topics in Medicinal Chemistry, 2009, Vol. 9, No. 16, 1494, which is hereby incorporated by reference.

### Example 42: Determination of the Anti-Proliferative Activity of Active Duocarmycin Drug Leads in RMS Cell Lines

Briefly, 180NL of a cell suspension (~2000 cells/well) was added in fresh, sterile 96-well plates. Afterwards, rhabdomyosarcoma (RMS) cell lines (RH30, RH41 and JR1) were incubated for 24 h at 37° C., 5% CO₂ to allow cellular attachment on the surface of the wells. On the next day after cell seeding, cells were treated with the duocarmycin compounds. 20 μL of drug dilution was added into the wells, making up a final volume of 200 μL/well. In untreated control wells, 20 μL of fresh medium was added instead (0.1% DMSO). Duocarmycin stock concentrations were prepared in DMSO; the concentration of the latter in the wells containing the medium and cells never exceeded 0.1% (v/v). The concentrations tested ranged from 100 μM to 1 pM depending on the IC50 of each compound from preliminary screening. Each experiment was tested at least in triplicate, and antiproliferative activity was determined after 96 hours using a standard MTT (3-(4,5-dimethylthiazol-2-yl)-2,5-diphenyltetrazolium bromide) assay. The resulting data is shown in Table 2 below.

TABLE 2

| | IC50 | | | | | |
|---|---|---|---|---|---|---|
| Compound | RH30 | | RH41 | | JR1 | |
| DP31 | 86.08 ± 24.82 | pM | 36.79 ± 31.61 | pM | 242.61 ± 307.55 | pM |
| DP40 | 227.41 ± 306.81 | nM | 530.00 ± 513.65 | nM | 1.40 ± 1.24 | μM |
| DP42 | 81.45 ± 77.30 | nM | 377.70 ± 209.22 | nM | 277.93 ± 156.16 | nM |
| DP51 | 1.22 ± 0.83 | μM | 2.51 ± 1.16 | μM | 18.54 ± 9.64 | μM |
| DP77 | 3.08 ± 2.57 | μM | 22.51 ± 17.38 | μM | 22.12 ± 19.63 | μM |
| DP78 | 267.93 ± 129.70 | nM | 499.95 ± 416.00 | μM | 3.51 ± 1.84 | μM |
| DP59 | 376.10 ± 157.07 | pM | 297.57 ± 154.26 | pM | 1.33 ± 1.93 | nM |
| DP63 | 100.94 ± 47.98 | pM | 20.40 ± 11.41 | pM | 217.48 ± 286.96 | pM |
| DP65 | 76.33 ± 129.36 | pM | 5.51 ± 2.29 | pM | 323.19 ± 291.14 | pM |
| DP70 | 18.90 ± 18.85 | nM | 31.18 ± 28.76 | nM | 44.61 ± 56.14 | nM |
| DP36 | 6.69 ± 4.33 | nM | 24.85 ± 33.96 | nM | 7.28 ± 6.10 | nM |
| DP44 | 2.19 ± 3.10 | μM | 2.80 ± 1.96 | μM | 4.86 ± 4.78 | μM |
| DP45 | 56.72 ± 37.56 | μM | 67.97 ± 50.68 | μM | 32.91 ± 29.06 | μM |
| DP47 | 60.27 ± 49.14 | nM | 19.16 ± 20.89 | nM | 59.16 ± 27.24 | nM |
| DP48 | 355.36 ± 352.24 | nM | 580.80 ± 647.52 | nM | 5.17 ± 4.28 | μM |
| ICT11038 | 35.79 ± 16.43 | nM | 120.81 ± 95.31 | μM | 801.13 ± 207.06 | μM |
| ICT11039 | 12.79 ± 8.46 | μM | 1.14 ± 0.50 | μM | 10.08 ± 5.78 | μM |
| ICT11043 | 56.02 ± 17.03 | μM | 30.18 ± 23.08 | μM | 137.61 ± 75.82 | μM |
| ICT11044 | 123.67 ± 134.12 | nM | 291.20 ± 204.26 | nM | 422.10 ± 176.53 | nM |
| ICT11040 | 493.87 ± 266.80 | nM | 575.90 ± 404.68 | nM | 860.70 ± 329.39 | nM |
| ICT11041 | 8.04 ± 1.03 | μM | 6.00 ± 5.12 | μM | 12.20 ± 4.58 | μM |

Further evidence of the applicability for the cores found in Formula IB to IJ can be found in the following articles: pyrroloindolone representative of the core of Formula IB, D. L. Boger, D. S. Johnson, Angew. Chem., Int. Ed. Engl., 35 (1996), p. 1438; pyrazoloindolone representative of the core of Formula IC, P. G. Baraldi, et al, Anti-Cancer Drug Des., 12 (1997), p. 67; Cyclopropylfurano[e]indolone representative of the core of Formula ID, F. Mohamadi et al, J. Med. Chem., 37 (1994), p. 232 and Patel et al. Org. Chem., 62 (1997), p. 8868; 1,2,9,9a-tetrahydropyrdo[3,2-e]indol-4-one representative of the core of Formula IJ, Boger, D. L.; Boyce, C. W., J. Org. Chem. 2000, 65, 4088-4100, and a core representative of Formula IK and IL, Mol. Cancer Ther. (2023) 22 (12), 1465-1478, all of which are hereby incorporated by reference.

### Example 43: Synthesis of Modified Payload for Conjugation to an Antibody to Give an ADC

Method for synthesis of AP-01 and AP-02: 5 Equivalents of tert-butyl methyl(2-(methylamino)ethyl)carbamate (TBMMEC) (171.4 μL, 0.87 mmol) and 10 equivalents of DMAP (212.9 mg, 1.74 mmol) were dissolved in 4 mL anhydrous CH₂Cl₂ and chilled to 0° C. under nitrogen. 2.5 equivalents of diphosgene (52.2 μL, 0.436 mmol) was added dropwise to the flask containing the amine, after which the solution was left to stir for 30 minutes at room temperature. In a separate flask, DP-36 (84 mg, 0.174 mmol) was dissolved in 2 mL of anhydrous THF under nitrogen. This solution was transferred to the first flask and left to react for 4 days under nitrogen. The reaction was quenched with cold water and left to stir for one hour. The product was extracted with CH₂Cl₂ (×3). The organic phase was collected, dried and evaporated under vacuum and the crude product was purified by flash chromatography (hexane/ethyl acetate 1:1) yielding the conjugate (85 mg, 70%) as a white solid.

### tert-Butyl (4-(1-(chloromethyl)-3-(5-methoxy-1H-indole-2-carbonyl)-2,3-dihydro-1H-benzo[e]indol-5-yl)phenyl) ethane-1,2-diylbis(methylcarbamate) (AP-01)

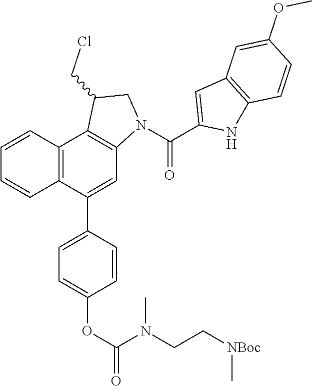 [SMILES: COc1ccc2[nH]c(C(=O)N3CC(CCl)c4c3cc(-c3ccc(OC(=O)N(C)CCN(C)C(=O)OC(C)(C)C)cc3)c3ccccc43)cc2c1]

¹H NMR (400 MHz, CDCl₃) δ 9.56 (s, 1H); 8.53 (s, 1H); 7.92 (d, 1H, J=8.4 Hz); 7.83 (d, 1H, J=8.4 Hz); 7.55 (ddd, 1H, J=1.2 Hz, J=6.8 Hz, J=8.4 Hz); 7.48 (d, 2H, J=8.8 Hz); 7.37 (dd, 1H, J=6.8 Hz, J=8.4 Hz); 7.32 (d, 1H, J=9.2 Hz); 7.23 (m, 2H); 7.12 (d, 1H, J=2.4 Hz); 7.05 (dd, 1H, J=1.2 Hz, J=2.4 Hz); 6.96 (dd, 1H, J=2.4 Hz, J=9.2 Hz); 4.87 (dd, 1H, J=1.6 Hz, J=10.8 Hz); 4.71 (dd, 1H, J=8.4 Hz, J=10.8 Hz); 4.24 (m, 1H); 4.04 (dd, 1H, J=3.2 Hz, J=11.2 Hz); 3.86 (s, 3H); 3.66-3.47 (m, 5H); 3.17+3.08 (2s, 3H); 2.96 (m, 3H); 1.48 (s, 9H); ES⁺ MS C₃₉H₄₁ClN₄O₆ (696.2) m/z (%) 697.4 [M+H]⁺ (20).

Method for synthesis of AP-03: Boc protected AP-01 (85 mg, 0.122 mmol) was dissolved in 7 mL CH₂Cl₂:TFA (6:1) on ice and stirred for 30 minutes. The solvent was removed under nitrogen stream while on ice. The resulting product (72 mg, 0.122 mmol) was dried under vacuum for complete removal of TFA, and further dissolved in 6 mL anhydrous DMF and 4 mL anhydrous CH₂Cl₂ and reacted with Fmoc-Val-Ala-PAB-PNP (165 mg, 0.24 mmol) and DMAP (30 mg, 0.24 mmol) and stirred at room temperature overnight. The organic solvent was evaporated under reduced pressure and the crude product was purified by flash column chromatography (CH₂Cl₂→CH₂Cl₂:MeOH 100:6) to afford AP-03 (100 mg, 72%) as a white solid.

### 4-((S)-2-((S)-2-((((9H-fluoren-9-yl)methoxy)carbonyl)amino)-3-methylbutanamido)propanamido)benzyl (4-(1-(chloromethyl)-3-(5-methoxy-1H-indole-2-carbonyl)-2,3-dihydro-1H-benzo[e]indol-5-yl)phenyl) ethane-1,2-diylbis(methylcarbamate) (AP-03)

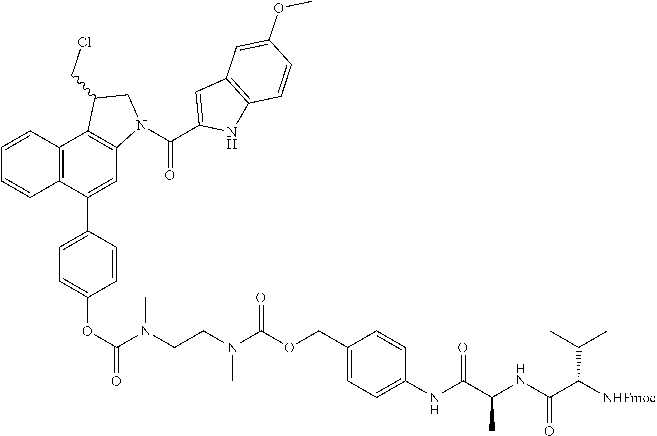

¹H NMR (400 MHz, (CD₃)₂CO) δ 10.9 (m, 1H); 9.3 (m, 1H); 8.53 (s, 1H); 8.05 (d, 1H, J=8.4 Hz); 7.84 (m, 3H); 7.71-7.65 (m, 4H); 7.59 (m, 1H); 7.52-7.27 (m, 11H); 7.22 (s, 1H); 7.18 (d, 1H, J=3.2 Hz); 7.17-7.09 (m, 1H); 6.93 (dt, 1H, J=2.4 Hz, J=9.2 Hz); 6.7 (m, 1H); 5.13-5.02 (m, 2H); 4.96-4.87 (m, 2H); 4.6-4.52 (m, 1H); 4.49-4.43 (m, 1H); 4.41-4.28 (m, 2H); 4.21 (t, 1H, J=6.8 Hz); 4.19-4.14 (m, 1H); 4.09 (m, 1H); 3.96 (dd, 1H, J=8.4 Hz, J=11.2 Hz); 3.82 (m, 3H); 3.68-3.51 (m, 4H); 3.14-2.96 (m, 6H); 2.15 (td, 1H, J=0.8 Hz, J=7.6 Hz); 1.33-1.19 (m, 3H); 0.92 (m, 6H); ES⁺ MS C₆₅H₆₄ClN₇O₁₀ (1138.6) m/z (%) 1139.4 [M+H]⁺ (100).

Method for synthesis of AP-04: AP-03 (100 mg, 0.088 mmol) was dissolved in 2 mL THF and 20 μL of DBU were added in the flask. The reaction was stirred for 3 minutes at room temperature, followed by neutralisation with 1 mL 95% acetic acid and dried by lyophilisation. The resulting crude was purified by column chromatography (CH₂Cl₂:MeOH:TEA 90:10:1). Compound AP-04 (35 mg, 43%) was obtained as a white solid.

### 4-((S)-2-((S)-2-amino-3-methylbutanamido)propanamido)benzyl (4-(1-(chloromethyl)-3-(5-methoxy-1H-indole-2-carbonyl)-2,3-dihydro-1H-benzo[e]indol-5-yl)phenyl) ethane-1,2-diylbis(methylcarbamate) (AP-04)

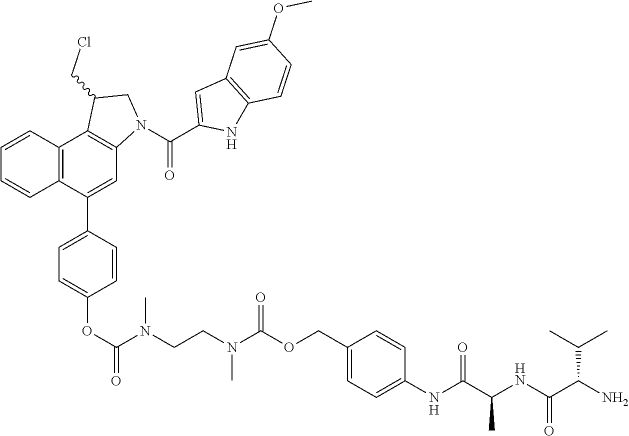 [SMILES: COc1ccc2[nH]c(C(=O)N3CC(CCl)c4c3cc(-c3ccc(OC(=O)N(C)CCN(C)C(=O)OCc5ccc(NC(=O)[C@H](C)NC(=O)[C@@H](N)C(C)C)cc5)cc3)c3ccccc43)cc2c1]

¹H NMR (400 MHz, CDCl₃) δ 8.97 (d, 1H, J=11.2 Hz); 8.48 (s, 1H); 7.91 (m, 2H); 7.83 (d, 1H, J=8 Hz); 7.57-7.48 (m, 3H); 7.42-7.21 (m, 6H); 7.14-7.09 (m, 2H); 7.06 (s, 1H); 7.0 (dd, 1H, J=2.4 Hz, J=9.2 Hz); 5.12-5.02 (m, 2H); 4.89 (d, 1H, J=10.8 Hz); 4.73 (m, 1H); 4.64 (m, 1H); 4.25 (m, 1H, CH); 4.05 (m, 1H); 3.87 (s, 3H); 3.7-3.47 (m, 5H); 3.3-2.98 (m, 7H); 2.27 (m, 1H); 1.31 (m, 3H); 0.96 (m, 3H); 0.82 (m, 3H); ES⁺ MS C₅₀H₅₄ClN₇O₈ (916.4) m/z (%) 917.3 [M+H]⁺ (100).

Method for synthesis of AP-05: Compound AP-04 (35 mg, 0.038 mmol) was dissolved in 1 mL DMF followed by the addition of DIPEA (16.6 NL, 0.095 mmol) and Mal-PEG4-NHS ester (29.4 mg, 0.057 mmol). The reaction was stirred at room temperature for one hour. The DMF was evaporated by nitrogen stream and purified by semipreparative HPLC, according to the method below.

Semipreparative HPLC purification method:

Solvent A: 90% water, 10% acetonitrile, 0.05% TFA

Solvent B: 90% acetonitrile, 10% water, 0.05% TFA

| Time (min) | Solvent A % | Solvent B % |
|---|---|---|
| 0.00 | 95 | 5 |
| 5.00 | 50 | 50 |
| 28.00 | 0 | 100 |
| 30.00 | 0 | 100 |
| 31.00 | 95 | 5 |
| 35.00 | 95 | 5 |

### 4-(1-(Chloromethyl)-3-(5-methoxy-1H-indole-2-carbonyl)-2,3-dihydro-1H-benzo[e]indol-5-yl)phenyl 4-((2S,5S)-25-(2,5-dioxo-2,5-dihydro-1H-pyrrol-1-yl)-5-isopropyl-2-methyl-4,7,23-trioxo-10,13,16,19-tetraoxa-3,6,22-triazapentacosanamido)benzyl ethane-1,2-diylbis(methylcarbamate) (AP-05)

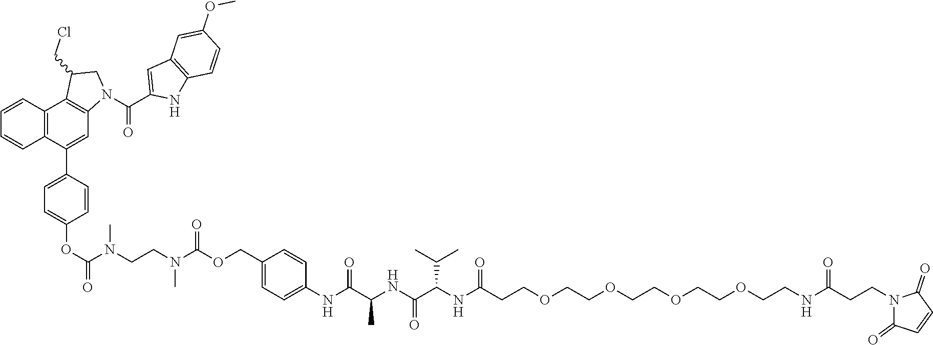 [SMILES: COc1ccc2[nH]c(C(=O)N3CC(CCl)c4c3cc(-c3ccc(OC(=O)N(C)CCN(C)C(=O)OCc5ccc(NC(=O)[C@H](C)NC(=O)[C@@H](NC(=O)CCOCCOCCOCCOCCNC(=O)CCN6C(=O)C=CC6=O)C(C)C)cc5)cc3)c3ccccc43)cc2c1]

¹H NMR (400 MHz, (CD₃)₂CO) δ 10.9 (bs, 1H); 9.22 (m, 1H); 8.53 (s, 1H); 8.07 (d, 1H, J=8.4 Hz); 7.89-7.82 (m, 1H); 7.74-7.69 (m, 3H); 7.6 (ddd, 1H, J=1.2 Hz, J=6.8 Hz, J=8.4 Hz); 7.52 (dd, 1H, J=3.6 Hz, J=9 Hz); 7.45-7.41 (m, 2H); 7.35 (m, 1H); 7.23 (bs, 1H); 7.19 (d, 1H, J=2.8 Hz); 7.17-7.12 (m, 2H); 6.95 (dd, 1H, J=2.4 Hz, J=9 Hz); 6.81 (s, 2H); 5.18-5.02 (m, 2H); 4.97-4.88 (m, 2H); 4.52-4.45 (m, 2H); 4.31-4.25 (m, 1H); 4.21-4.15 (m, 1H); 3.97 (m, 1H); 3.83 (s, 3H); 3.73 (d, 2H, J=7.6 Hz); 3.71 (d, 2H, J=6.4 Hz); 3.69-3.51 (m, 16H); 3.47 (t, 2H, J=5.6 Hz); 3.3 (q, 2H, J=5.6 Hz); 3.08-2.98 (m, 6H); 2.55 (m, 2H); 2.45 (t, 2H, J=7.4 Hz); 2.12 (m, 1H); 1.29-1.19 (m, 3H); 0.92 (m, 6H); ES⁺ MS C₆₈H₈₀ClN₉O₁₆ (1314.8) m/z (%) 680.1 [M+2Na]²⁺ (100).

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A compound of formula I, or a pharmaceutically acceptable salt thereof: 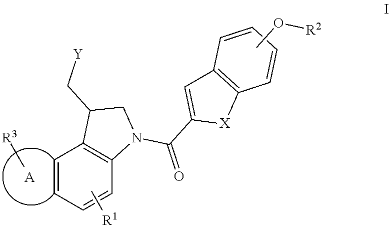
wherein:
A represents a 5 or 6 membered ring wherein there are 3 or 4 atoms not represented in the ring and the 3 or 4 atoms are selected as follows: 0, 1 or 2 of the atoms are selected independently from C═O, N, NH, S, or O with the balance of the atoms being made up of CH;
X represents NH, S or O;
Y represents a halo group;
R¹ represents a group selected from: —OR^{4a}, —NR^{4a}R^{4b}, substituted or unsubstituted C₁₋₆ alkyl, substituted or unsubstituted C₅₋₁₀ cycloalkyl, substituted or unsubstituted C₅₋₁₀ heterocycloalkyl, substituted or unsubstituted C₆₋₁₀ aryl, and substituted or unsubstituted C₅₋₁₀ heteroaryl,
wherein, when substituted, the R¹ group is substituted with 1, 2, or 3 groups independently selected from: halo, —OR^{4a}, —NR^{4a}R^{4b}, —NO₂, C₁₋₆ alkyl, C₁₋₆ haloalkyl, C₁₋₆ alkyl substituted with OH, C₁₋₆ alkyl substituted with OMe, and C₁₋₆ alkyl substituted with ═O;
R² represents a group selected from: H, C₁₋₆ alkyl, C₂₋₆ alkenyl, C₂₋₆ alkynyl, and —C(O)—R⁵;
R³ represents a group selected from: H, —OH, halo, C₁₋₆ alkyl, C₁₋₆ haloalkyl, C₁₋₆ alkoxy, —NR^{6a}R^{6b}, C₁₋₆ alkoxy substituted with C₆₋₁₀ aryl and C₁₋₆ alkyl substituted with C₆₋₁₀ aryl;
R^{4a} and R^{4b} are each independently selected from: C₁₋₆ alkyl, C₂₋₆ alkenyl, C₂₋₆ alkynyl, C₁₋₆ alkyl substituted with OH, C₁₋₆ alkyl substituted with OMe, C₁₋₆ alkyl substituted with ═O, C₁₋₆ alkyl substituted with a substituted or unsubstituted C₆₋₁₀ aryl, —C(O)—C₁₋₆alkyl, —(CR^{7a}R^{7b})ₙC(O)OR⁸, —(CR^{7a}R^{7b})ₙC(O)NHR⁸, substituted or unsubstituted C₅₋₁₀ cycloalkyl, substituted or unsubstituted C₅₋₁₀ heterocycloalkyl, substituted or unsubstituted C₆₋₁₀ aryl, and substituted or unsubstituted C₅₋₁₀ heteroaryl;
or R^{4b} is H;
wherein, when substituted, the R^{4a} and R^{4b} group is substituted with 1 or 2 groups selected from: halo, OH, —NO₂, C₁₋₆ alkyl, C₁₋₆ alkoxy, C₁₋₆ haloalkyl, C₁₋₆ alkyl substituted with OH, C₁₋₆ alkyl substituted with OMe, C₁₋₆ alkyl substituted with ═O, and —NH₂;
n is 1, 2, or 3 (preferably 1);
R⁵ represents a group selected from: C₁₋₆ alkyl, C₂₋₆ alkenyl, and C₂₋₆ alkynyl;
R^{6a} and R^{6b} are each independently selected from: H and C₁₋₆ alkyl;
one of R^{7a} and R^{7b} is a group selected from: H, C₁₋₆ alkyl and C₁₋₆ alkyl substituted with —OH, —SH, —SMe, —NH₂, —C(O)OH, —C(O)NH₂, guanidine, C₆₋₁₀ aryl, C₆₋₁₀ aryl substituted with —OH, or C₅₋₁₀ heteroaryl; and all other R^{7′} and R^{7b} are H; and
R⁸ represents a group selected from: H and C₁₋₆ alkyl;
provided that R¹ is not —OBn when R² is methyl.

2. The compound of claim 1 wherein the compound is a compound with a Formula selected from:

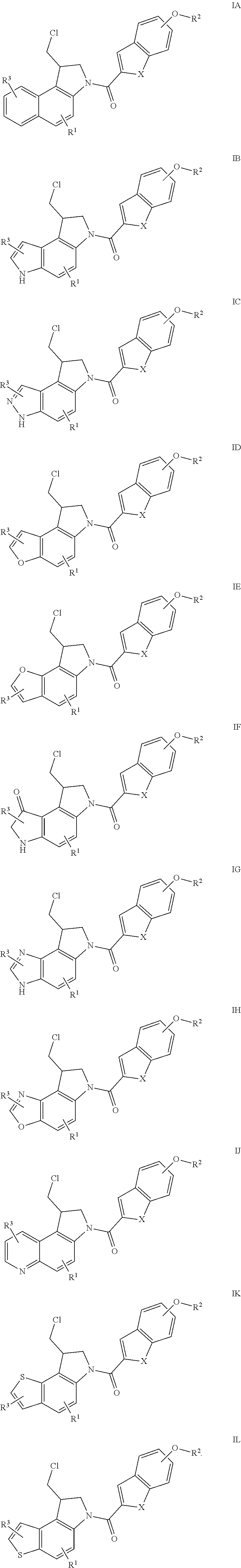

3. The compound of claim 1 wherein the compound is a compound of Formula IA:

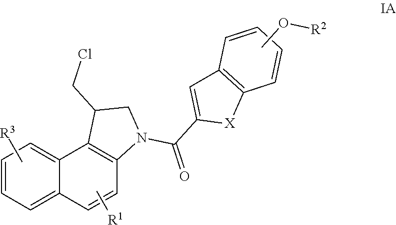

4. The compound of claim 1, wherein R³ represents a group selected from: H, —OH, C₁₋₆ alkyl, C₁₋₆ alkoxy, —NR^{6a}R^{6b}, an C₁₋₆ alkoxy substituted with C₆₋₁₀ aryl.

5. The compound of claim 4, wherein R³ represents a group selected from: H, —OH, methyl, ethyl, —OMe, —NH₂, —NHMe, —N(Me)₂, and —OBn.

6. The compound of claim 1, wherein R² represents a group selected from: C₁₋₆ alkyl and —C(O)—R⁵.

7. (canceled)

8. The compound of claim 1, wherein the compound is a compound according to formulae Vila or VIIb:

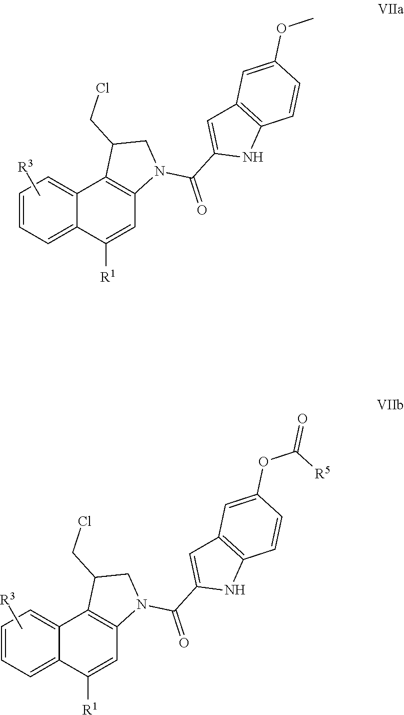

9. The compound of claim 1, wherein R¹ represents a group selected from: —OR^{4a}, —NR^{4a}R^{4b}, substituted or unsubstituted C₅₋₁₀ heterocycloalkyl, substituted or unsubstituted C₆₋₁₀ aryl and substituted or unsubstituted C₅₋₁₀ heteroaryl,
wherein, when substituted, the R¹ group is substituted with 1, 2, or 3 groups (optionally 1 or 2 groups) independently selected from: halo, OR^{4a}, —NR^{4a}R^{4b}, —NO₂, C₁₋₆ alkyl, C₁₋₆ alkyl substituted with OH, and C₁₋₆ alkyl substituted with ═O.

10. The compound of claim 1, wherein R^{4a} and R^{4b} are each independently selected from: C₁₋₆ alkyl, C₁₋₆ alkyl substituted with OH, C₁₋₆ alkyl substituted with OMe, C₁₋₆ alkyl substituted with C₆₋₁₀ aryl, —(CR^{7a}R^{7b})ₙC(O)OR⁸, —(CR^{7a}R^{7b})ₙC(O)NHR⁸, substituted or unsubstituted C₅₋₁₀ cycloalkyl, substituted or unsubstituted C₅₋₁₀ heterocycloalkyl, and substituted or unsubstituted C₆₋₁₀ aryl;
or R^{4b} is H;
wherein, when substituted, the R^{4a} and R^{4b} group is substituted with 1 or 2 groups selected from: halo, OH, —NO₂, C₁₋₆ alkyl, C₁₋₆ alkoxy, C₁₋₆ haloalkyl, C₁₋₆ alkyl substituted with OH, C₁₋₆ alkyl substituted with OMe, C₁₋₆ alkyl substituted with ═O, and —NH₂ (optionally halo, —NO₂, C₁₋₆ alkyl, C₁₋₆ alkoxy, and C₁₋₆ haloalkyl).

11. The compound of claim 1, wherein R^{7a} and R^{7b} is a group selected from: H, C₁₋₆ alkyl and C₁₋₆ alkyl substituted with —OH, —SH, —SMe, —NH₂, —C(O)OH, —C(O)NH₂, guanidine, benzene, phenol, indole or imidazole, and all other R^{7a} and R^{7b} are H.

12. The compound of claim 1, wherein n is 1.

13. The compound of claim 1, wherein R¹ is selected from: —OH (optionally provided that R² is not H) OBn, —N(Et)₂, —NHBu, and:

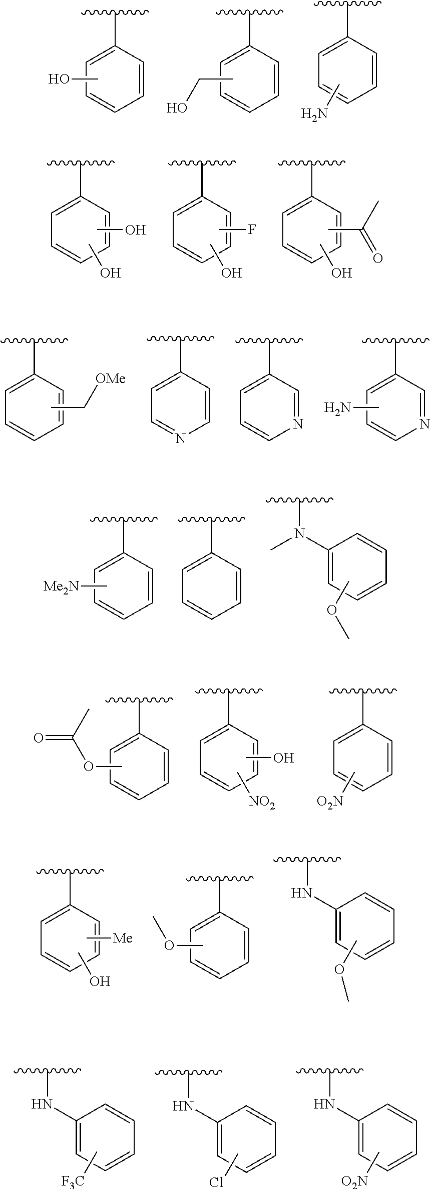

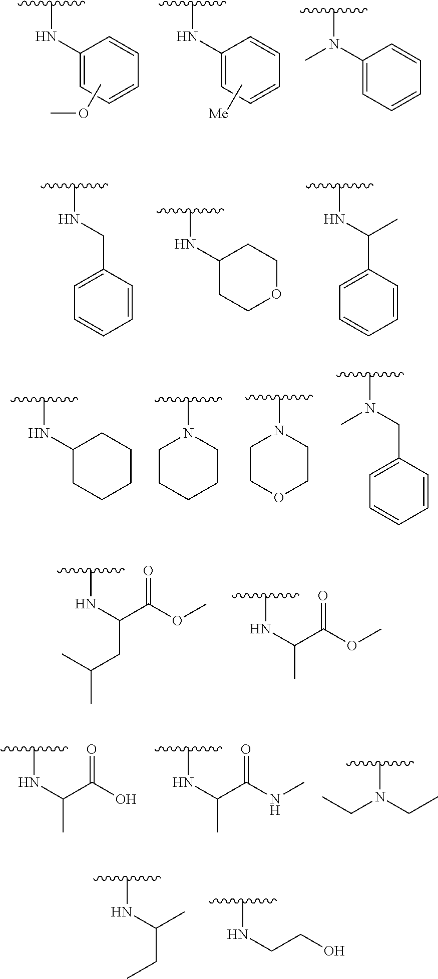

14. The compound of claim 1, wherein the compound of Formula I is selected from:

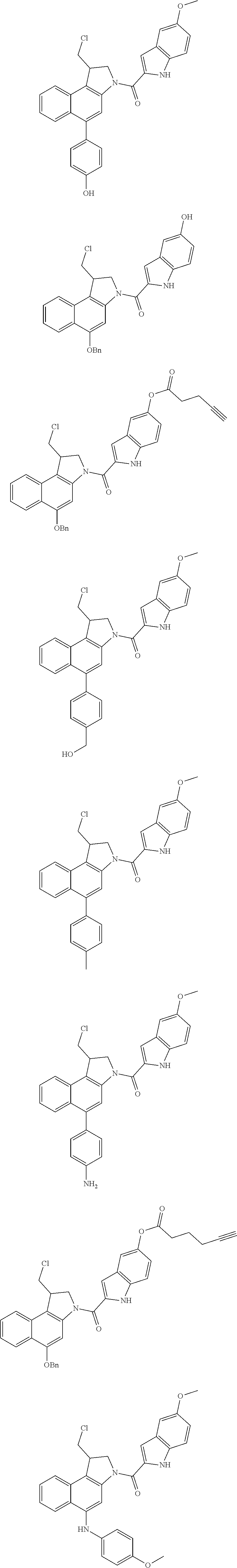

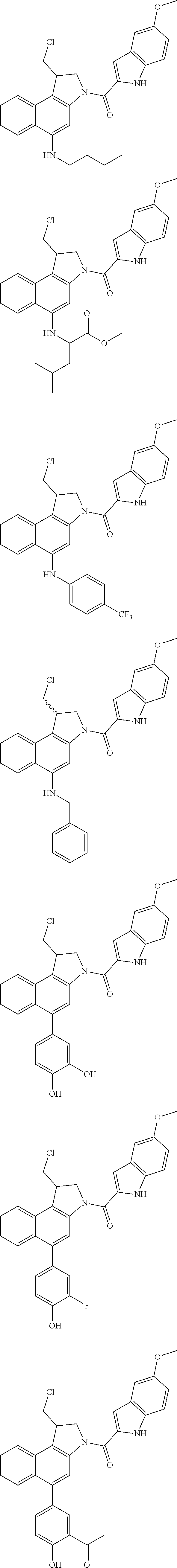

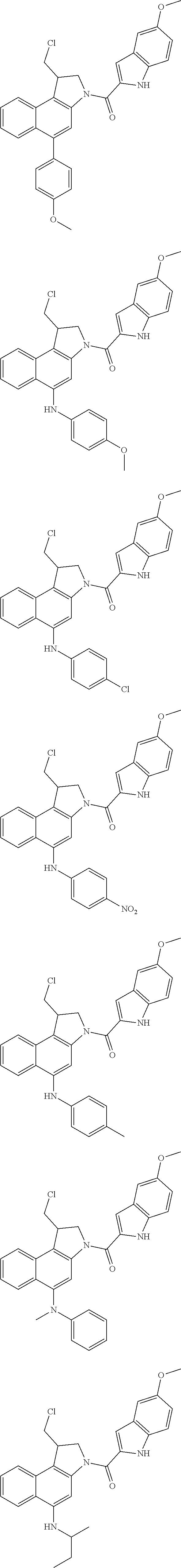

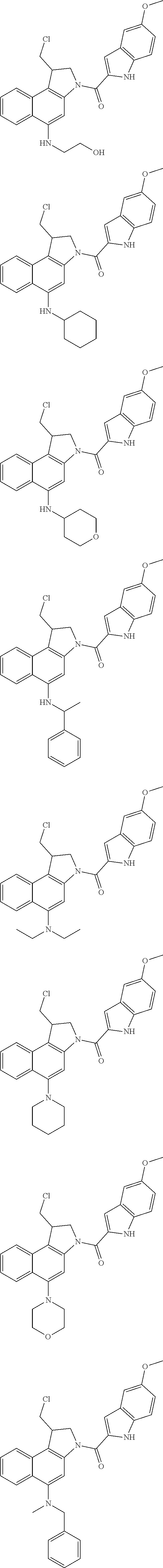

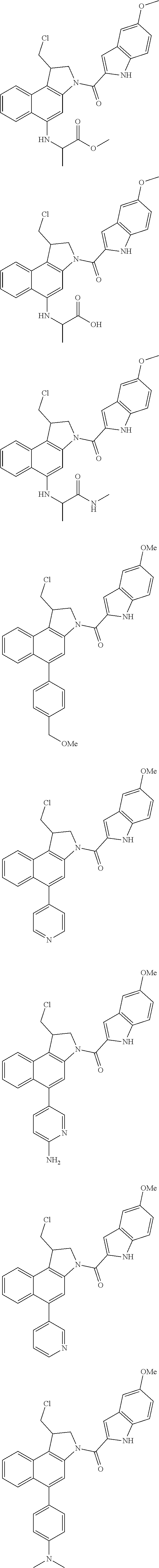

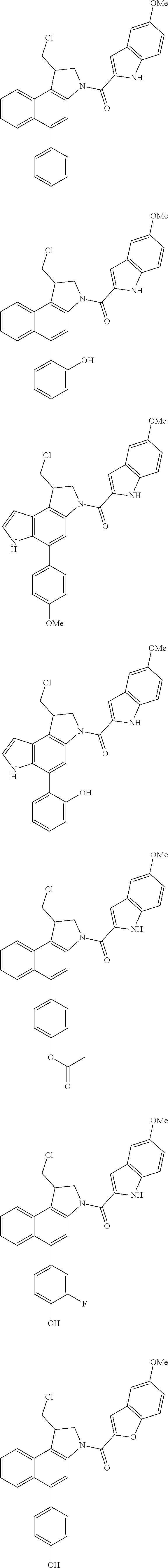

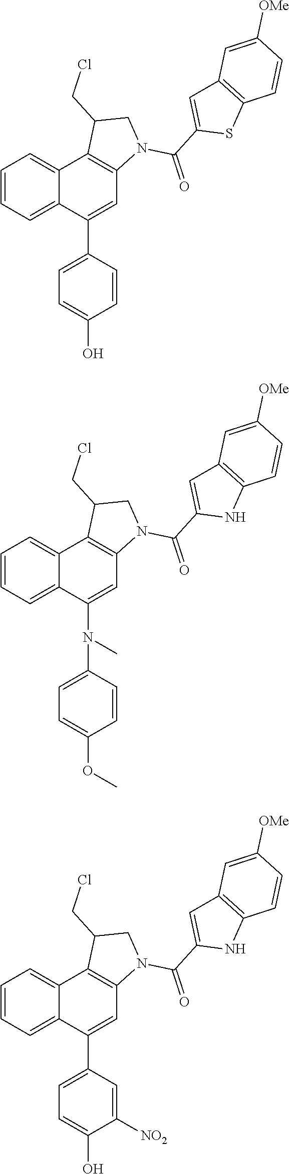 [SMILES: COc1ccc2sc(C(=O)N3CC(CCl)c4c3cc(-c3ccc(O)cc3)c3ccccc43)cc2c1 ; COc1ccc(N(C)c2cc3c(c4ccccc24)C(CCl)CN3C(=O)c2cc3cc(OC)ccc3[nH]2)cc1 ; COc1ccc2[nH]c(C(=O)N3CC(CCl)c4c3cc(-c3ccc(O)c([N+](=O)[O-])c3)c3ccccc43)cc2c1]

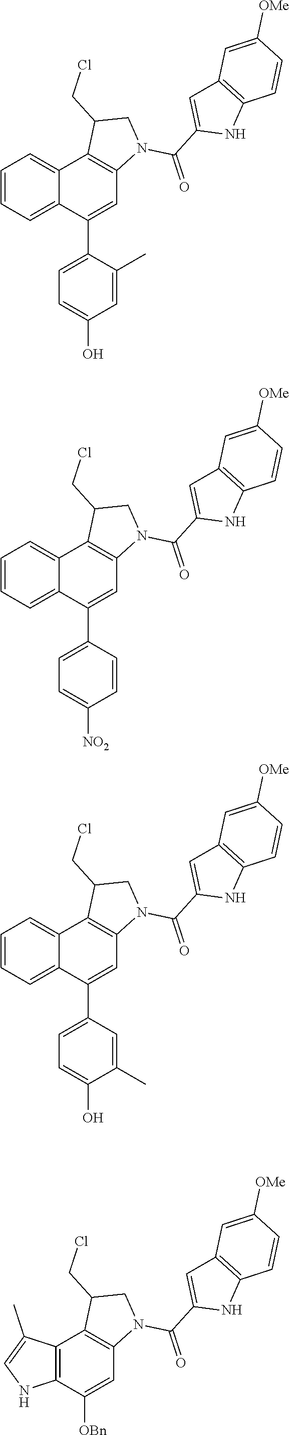 [SMILES: COc1ccc2[nH]c(C(=O)N3CC(CCl)c4c3cc(-c3ccc(O)cc3C)c3ccccc43)cc2c1 ; COc1ccc2[nH]c(C(=O)N3CC(CCl)c4c3cc(-c3ccc([N+](=O)[O-])cc3)c3ccccc43)cc2c1 ; COc1ccc2[nH]c(C(=O)N3CC(CCl)c4c3cc(-c3ccc(O)c(C)c3)c3ccccc43)cc2c1 ; COc1ccc2[nH]c(C(=O)N3CC(CCl)c4c3cc(OCc3ccccc3)c3[nH]cc(C)c43)cc2c1]

15. (canceled)

16. (canceled)

17. A method for the treatment of cancer, wherein the method comprises administering a pharmaceutically effective amount of a compound of claim 1.

18. The method of claim 17, wherein the cancer is selected from breast, lung, prostate, colon, bladder, brain, pancreas, head & neck and neuroblastoma.

19. A use of the compound of claim 1, in the manufacture of an antibody-drug conjugate.

20. An antibody-drug conjugate, wherein the drug is a compound of claim 1.

21. The antibody-drug conjugate of claim 20, wherein the antibody-drug conjugate further comprises an antibody, biosimilar, affimer or other non-antibody binding proteins, and a linker connecting the drug to the antibody.
